SEQUENCE LISTING (55 sequences)

SEQ ID NO: 1 - DNA, 19 bp, Human papillomavirus
ccagaaaguuaccacaguu

SEQ ID NO: 2 - DNA, 19 bp, Human papillomavirus
ccggaaaguuaccacaguu

SEQ ID NO: 3 - DNA, 19 bp, Human papillomavirus
aacugugguaacuuucugg

SEQ ID NO: 4 - DNA, 19 bp, Human papillomavirus
aacugugguaacuuuccgg

SEQ ID NO: 5 - DNA, 21 bp, Human papillomavirus
guuaccacaguuaugcacaga

SEQ ID NO: 6 - DNA, 21 bp, Human papillomavirus
ucugugcauaacugugguaac

SEQ ID NO: 7 - DNA, 11 bp, Artificial Sequence
  Cassette
guucaagaccc

SEQ ID NO: 8 - DNA, 9 bp, Artificial Sequence
  Cassette
uucaagacc

SEQ ID NO: 9 - DNA, 19 bp, Human papillomavirus
ccggaaagttaccacagtt

SEQ ID NO: 10 - DNA, 21 bp, Human papillomavirus
gttaccacagttatgcacaga

SEQ ID NO: 11 - DNA, 19 bp, Human papillomavirus
ccagaaagttaccacagtt

SEQ ID NO: 12 - DNA, 139 bp, Artificial Sequence
  Cassette
ggatccttccgggctgatgagtccaattggacgaaacggtactcgagtaccgtccccggaaagttaccacagttgttgacggagaattctccgtcctgatgagtccggccggacgaaacaactgagatctttttctaga

SEQ ID NO: 13 - DNA, 139 bp, Artificial Sequence
  Cassette
ggatccacagttgctgatgagtccaattggacgaaacggtactcgagtaccgtccaactgtggtaactttccgggttgacggagaattctccgtcctgatgagtccggccggacgaaacccggaagatctttttctaga

SEQ ID NO: 14 - DNA, 139 bp, Artificial Sequence
  Cassette
ggatcctggtaacctgatgagtccaattggacgaaacggtactcgagtaccgtcgttaccacagttatgcacagattgacggagaattctccgtcctgatgagtccggccggacgaaatctgtgagatctttttctaga

SEQ ID NO: 15 - DNA, 139 bp, Artificial Sequence
  Cassette
ggatccgcacagactgatgagtccaattggacgaaacggtactcgagtaccgtctctgtgcataactgtggtaacttgacggagaattctccgtcctgatgagtccggccggacgaaagttaccagatctttttctaga

SEQ ID NO: 16 - DNA, 178 bp, Artificial Sequence
  Cassette
ggatcagcttcgagctctgatgagtccgtgaggacgaaacggtacccggtaccgtcagctcgacctcagatccccagaaagttaccacagttgttaattgatccgtcgacggatgtagatccgtcctgatgagtccgtgaggacgaaacggatctgcagcggatgatctttttctaga

SEQ ID NO: 17 - DNA, 178 bp, Artificial Sequence
  Cassette
ggatcagcttcgagctctgatgagtccgtgaggacgaaacggtacccggtaccgtcagctcgacctcagatccaactgtggtaactttctgggttaattgatccgtcgacggatgtagatccgtcctgatgagtccgtgaggacgaaacggatctgcagcggatgatctttttctaga

SEQ ID NO: 18 - DNA, 21 bp, Human papillomavirus
tttggagctactgtggagtta

SEQ ID NO: 19 - DNA, 139 bp, Artificial Sequence
  Cassette
aagcttctccaaactgatgagtccaattggacgaaacggtactcgagtaccgtctttggagctactgtggagttattgacggagaattctccgtcctgatgagtccggccggacgaaataactcagatctttttctaga

SEQ ID NO: 20 - DNA, 139 bp, Artificial Sequence
  Cassette
aagcttggagttactgatgagtccaattggacgaaacggtactcgagtaccgtctaactccacagtagctccaaattgacggagaattctccgtcctgatgagtccggccggacgaaatttggaagatctttttctaga

SEQ ID NO: 21 - DNA, 183 bp, Artificial Sequence
  Cassette
ggatccttctgggctgatgagtccaattggacgaaacgatgacattctggtactcgagtaccagaatgtcatcgtccccagaaagttaccacagttgttgagcgcaacgacgcggagaattctccgcgtcgttgcgctcctgatgagtccggccggacgaaacaactgagatctttttctaga

SEQ ID NO: 22 - DNA, 183 bp, Artificial Sequence
  Cassette
ggatccacagttgctgatgagtccaattggacgaaacgatgacattctggtactcgagtaccagaatgtcatcgtccaactgtggtaactttctgggttgagcgcaacgacgcggagaattctccgcgtcgttgcgctcctgatgagtccggccggacgaaacccagaagatctttttctaga

SEQ ID NO: 23 - DNA, 252 bp, Artificial Sequence
  Cassette
ggatcatccagctttggaaccctgatgagtccgtgaggacgaaacgatgacattctgctgaccagattcacggtcagcagaatgtcatcgtcggttccaggatccccagaaagttaccacagttgttaattccaagggtctgcgcaacgacgacgatgaggtaccacatcgtcgtcgttgcgcactgatgaggccgtgaggccgaaacccttgacgcgttcctatgcggccgctctaggatctttttctaga

SEQ ID NO: 24 - DNA, 252 bp, Artificial Sequence
  Cassette
ggatcatccagctttggaaccctgatgagtccgtgaggacgaaacgatgacattctgctgaccagattcacggtcagcagaatgtcatcgtcggttccaggatccaactgtggtaactttctgggttaattccaagggtctgcgcaacgacgacgatgaggtaccacatcgtcgtcgttgcgcactgatgaggccgtgaggccgaaacccttgacgcgttcctatgcggccgctctaggatctttttctaga

SEQ ID NO: 25 - DNA, 263 bp, Artificial Sequence
  Cassette
ggatccttccgggctgatgagtccaattggacgaaacggtactcgagtaccgtccccggaaagttaccacagttgttgacggagaattctccgtcctgatgagtccggccggacgaaacaactgagatccacagttgctgatgagtccaattggacgaaacggtactcgagtaccgtccaactgtggtaactttccgggttgacggagaattctccgtcctgatgagtccggccggacgaaacccggaagatctttttctaga

SEQ ID NO: 26 - DNA, 270 bp, Artificial Sequence
  Cassette
ggatcctggtaacgctgatgagtccaattggacgaaacggtactcgagtaccgtccgttaccacagttatgcacagagttgacggagaattctccgtcctgatgagtccggccggacgaaatctgtgagatccgcacagagctgatgagtccaattggacgaaacggtactcgagtaccgtcctctgtgcataactgtggtaacgttgacggagaattctccgtcctgatgagtccggccggacgaaacgttaccagatctttttctaga

SEQ ID NO: 27 - DNA, 259 bp, Artificial Sequence
  Cassette
aagcttctccaaactgatgagtccaattggacgaaacggtactcgagtaccgtctttggagctactgtggagttattgacggagaattctccgtcctgatgagtccggccggacgaaataactcatggagttactgatgagtccaattggacgaaacggtactcgagtaccgtctaactccacagtagctccaaattgacggagaattctccgtcctgatgagtccggccggacgaaatttggaagatctttttctaga

SEQ ID NO: 28 - DNA, 265 bp, Homo sapiens
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacgaaacacc

SEQ ID NO: 29 - DNA, 670 bp, Mus musculus
agcttgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcactgtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgagtcgacggatccgggctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaaccccccggatctcgaagctt

SEQ ID NO: 30 - DNA, 238 bp, Artificial Sequence
  Cassette
ggatcagcttcgagctctgatgagtccgtgaggacgaaacggtacccggtaccgtcagctcgacctcagatcgacccagaaagttaccacagttgacggatgtagatccgtcctgatgagtccgtgaggacgaaactgtggtaactttctgggtcaattgatccgtcgacggatgtagatccgtcctgatgagtccgtgaggacgaaacggatctgcagcggatgatctttttctaga

SEQ ID NO: 31 - DNA, 312 bp, Artificial Sequence
  Cassette
ggatcatccagctttggaaccctgatgagtccgtgaggacgaaacgatgacattctgctgaccagattcacggtcagcagaatgtcatcgtcggttccaggatcgacccagaaagttaccacagttgacggatgtagatccgtcctgatgagtccgtgaggacgaaactgtggtaactttctgggtcaattccaagggtctgcgcaacgacgacgatgaggtaccacatcgtcgtcgttgcgcactgatgaggccgtgaggccgaaacccttgacgcgttcctatgcggccgctctaggatctttttctaga

SEQ ID NO: 32 - DNA, 238 bp, Artificial Sequence
  Cassette
ggatcagcttcgagctctgatgagtccgtgaggacgaaacggtacccggtaccgtcagctcgacctcagatcaagttaccacagttatgcacttgacggatgtagatccgtcctgatgagtccgtgaggacgaaagtgcataactgtggtaacttaattgatccgtcgacggatgtagatccgtcctgatgagtccgtgaggacgaaacggatctgcagcggatgatctttttctaga

SEQ ID NO: 33 - DNA, 312 bp, Artificial Sequence
  Cassette
ggatcatccagctttggaaccctgatgagtccgtgaggacgaaacgatgacattctgctgaccagattcacggtcagcagaatgtcatcgtcggttccaggatcaagttaccacagttatgcacttgacggatgtagatccgtcctgatgagtccgtgaggacgaaagtgcataactgtggtaacttaattccaagggtctgcgcaacgacgacgatgaggtaccacatcgtcgtcgttgcgcactgatgaggccgtgaggccgaaacccttgacgcgttcctatgcggccgctctaggatctttttctaga

SEQ ID NO: 34 - DNA, 364 bp, Artificial Sequence
  Cassette
aagcttcgagctctgatgagtccgtgaggacgaaacggtacccggtaccgtcagctcgacctcagatctctcgagcaattgatccgtcgacggatgtagatccgtcctgatgagtccgtgaggacgaaacggatctgcagcggatatccagctttggaaccctgatgagtccgtgaggacgaaacgatgacattctgctgaccagattcacggtcagcagaatgtcatcgtcggttccaggatccttgcctgaattccaagggtctgcgcaacgacgacgatgaggtaccacatcgtcgtcgttgcgcactgatgaggccgtgaggccgaaacccttgacgcgttcctatgcggccgctctaga

SEQ ID NO: 35 - DNA, 531 bp, Artificial Sequence
  Cassette
ggatcagcttcgagctctgatgagtccgtgaggacgaaacggtacccggtaccgtcagctcgacctcagatcgacccagaaagttaccacagttgacggatgtagatccgtcctgatgagtccgtgaggacgaaactgtggtaactttctgggtcaattgatccgtcgacggatgtagatccgtcctgatgagtccgtgaggacgaaacggatctgcagcggatatccagctttggaaccctgatgagtccgtgaggacgaaacgatgacattctgctgaccagattcacggtcagcagaatgtcatcgtcggttccaggatcgacccagaaagttaccacagttgacggatgtagatccgtcctgatgagtccgtgaggacgaaactgtggtaactttctgggtcaattccaagggtctgcgcaacgacgacgatgaggtaccacatcgtcgtcgttgcgcactgatgaggccgtgaggccgaaacccttgacgcgttcctatgcggccgctctaggatctttttctaga

SEQ ID NO: 36 - DNA, 531 bp, Artificial Sequence
  Cassette
ggatcagcttcgagctctgatgagtccgtgaggacgaaacggtacccggtaccgtcagctcgacctcagatcaagttaccacagttatgcacttgacggatgtagatccgtcctgatgagtccgtgaggacgaaagtgcataactgtggtaacttaattgatccgtcgacggatgtagatccgtcctgatgagtccgtgaggacgaaacggatctgcagcggatatccagctttggaaccctgatgagtccgtgaggacgaaacgatgacattctgctgaccagattcacggtcagcagaatgtcatcgtcggttccaggatcaagttaccacagttatgcacttgacggatgtagatccgtcctgatgagtccgtgaggacgaaagtgcataactgtggtaacttaattccaagggtctgcgcaacgacgacgatgaggtaccacatcgtcgtcgttgcgcactgatgaggccgtgaggccgaaacccttgacgcgttcctatgcggccgctctaggatctttttctaga

SEQ ID NO: 37 - DNA, 91 bp, Artificial Sequence
  Cassette
gatcaatttggagctactgtggagttgacggatgtagatccgtcctgatgagtccgtgaggacgaaactccacagtagctccaaattaatt

SEQ ID NO: 38 - DNA, 514 bp, Artificial Sequence
  Cassette
aagcttcgagctctgatgagtccgtgaggacgaaacggtacccggtaccgtcagctcgacctcagatcaatttggagctactgtggagttgacggatgtagatccgtcctgatgagtccgtgaggacgaaactccacagtagctccaaattaattgatccgtcgacggatgtagatccgtcctgatgagtccgtgaggacgaaacggatctgcagcggatatccagctttggaaccctgatgagtccgtgaggacgaaacgatgacattctgctgaccagattcacggtcagcagaatgtcatcgtcggttccaggatcaatttggagctactgtggagttgacggatgtagatccgtcctgatgagtccgtgaggacgaaactccacagtagctccaaattaattccaagggtctgcgcaacgacgacgatgaggtaccacatcgtcgtcgttgcgcactgatgaggccgtgaggccgaaacccttgacgcgttcctatgcggccgctctaga

SEQ ID NO: 39 - DNA, 169 bp, Artificial Sequence
  Cassette
ggatccttccgggctgatgagtccaattggacgaaacggtactcgagtaccgtccccggaaagttaccacagttgttcaagacccaactgtggtaactttccgggttgacggagaattctccgtcctgatgagtccggccggacgaaacccggaagatctttttctaga

SEQ ID NO: 40 - DNA, 169 bp, Artificial Sequence
  Cassette
ggatcctggtaacctgatgagtccaattggacgaaacggtactcgagtaccgtcgttaccacagttatgcacagattcaagacctctgtgcataactgtggtaacttgacggagaattctccgtcctgatgagtccggccggacgaaagttaccagatctttttctaga

SEQ ID NO: 41 - DNA, 169 bp, Artificial Sequence
  Cassette
aagcttctccaaactgatgagtccaattggacgaaacggtactcgagtaccgtctttggagctactgtggagttattcaagacctaactccacagtagctccaaattgacggagaattctccgtcctgatgagtccggccggacgaaatttggaagatctttttctaga

SEQ ID NO: 42 - DNA, 319 bp, Artificial Sequence
  Cassette
aagcttctccaaactgatgagtccaattggacgaaacggtactcgagtaccgtctttggagctactgtggagttattcaagacctaactccacagtagctccaaattgacggagaattctccgtcctgatgagtccggccggacgaaatttggaatctccaaactgatgagtccaattggacgaaacggtactcgagtaccgtctttggagctactgtggagttattcaagacctaactccacagtagctccaaattgacggagaattctccgtcctgatgagtccggccggacgaaatttggaagatctttttctaga

SEQ ID NO: 43 - DNA, 22 bp, Human papillomavirus
gacccagaaaguuaccacaguu

SEQ ID NO: 44 - DNA, 22 bp, Human papillomavirus
aacugugguaacuuucuggguc

SEQ ID NO: 45 - DNA, 22 bp, Human papillomavirus
gacccggaaaguuaccacaguu

SEQ ID NO: 46 - DNA, 22 bp, Human papillomavirus
aacugugguaacuuuccggguc

SEQ ID NO: 47 - DNA, 20 bp, Human papillomavirus
aaguuaccacaguuaugcac

SEQ ID NO: 48 - DNA, 20 bp, Human papillomavirus
gugcauaacugugguaacuu

SEQ ID NO: 49 - DNA, 23 bp, Human hepatitis virus B
gaauuuggagcuacuguggaguu

SEQ ID NO: 50 - DNA, 23 bp, Human hepatitis virus B
aacuccacaguagcuccaaauuc

SEQ ID NO: 51 - DNA, 22 bp, Human hepatitis virus B
aauuuggagcuacuguggaguu

SEQ ID NO: 52 - DNA, 22 bp, Human hepatitis virus B
aacuccacaguagcuccaaauu

SEQ ID NO: 53 - DNA, 21 bp, Human hepatitis virus B
uuuggagcuacuguggaguua

SEQ ID NO: 54 - DNA, 21 bp, Human hepatitis virus B
uaacuccacaguagcuccaaa

SEQ ID NO: 55 - DNA, 8 bp, Artificial Sequence
  Cassette
ttctagaa

(19) United States
(12) Patent Application Publication
(10) Pub. No.: US 2006/0269530 A1
(43) Pub. Date: Nov. 30, 2006

(54) RNA interference compositions and methods

(75) Inventors: Gary A. Clawson, Bathesda, MD (US); Wei-Hua Pan, Hummelstown, PA (US); Ping Xin, Hummelstown, PA (US)

Correspondence Address: Janis K. Fraser;FISH & RICHARDSON, 225 Franklin Street, Boston, MA, 02110-2804, US

(73) Assignee: THE PENN STATE RESEARCH FOUNDATION, University Park, PA (US)

(21) Appl. No.: 10/552,914

(22) PCT Filed: Feb. 23, 2004
(86) PCT No.: PCT/US04/05400

§ 371 Date: Oct. 13, 2005

Related U.S. Application Data
(60) Provisional application No. 60/449,066, filed on Feb. 21, 2003.

(51) Int. Cl. A61K 48/00; C07H 21/02; C12N 5/08; C12N 15/86; C12N 7/00

(52) U.S. Cl. 424/93.21; 514/44; 435/366; 435/235.1; 435/456; 536/23.1

(57) ABSTRACT

The invention provides isolated nucleic acids. For example, the invention provides isolated nucleic acids having at least one strand with both sense and antisense sequences that are complementary to each other. The invention also provides isolated nucleic acids having at least one strand that is a template for both sense and antisense sequences that are complementary to each other. In addition, the invention provides cells, viruses, and transgenic animals (e.g., transgenic non-human animals) containing one or more of the isolated nucleic acids provided herein as well as methods for using one or more of the isolated nucleic acids provided herein to reduce the level of an RNA (e.g., an mRNA) within a cell.

### BACKGROUND

1. Technical Field

The invention relates to methods and materials involved in RNA interference. In particular, the invention relates to nucleic acids that form RNA molecules that induce RNA interference as well as methods for using the nucleic acids to reduce the level of RNA (e.g., mRNA) within a cell.

2. Background Information

RNA interference, also known as gene silencing, typically employs small RNA molecules, called small interfering RNAs (siRNAs), to down-regulate the expression of targeted sequences in cells. siRNAs are double stranded molecules, one strand of which can be complementary to an mRNA. When an siRNA contains a sequence complementary to an mRNA, that mRNA is post-transcriptionally degraded by an RNA-Induced Silencing Complex (RISC) present within the cell (Hannon et al., Nature, 404:293-296 (2000)), thus effectively down-regulating expression of the associated gene. RNA interference has been reported to be several orders of magnitude more efficient than antisense technologies or targeted ribozyme technologies in the down-regulation of gene expression (Elbashir et al., Nature, 411:428-429 (2001)).

### SUMMARY

The invention provides isolated nucleic acids having at least one strand with both sense and antisense sequences that are complementary to each other. The invention also provides isolated nucleic acids having at least one strand that is a template for both sense and antisense sequences that are complementary to each other. In each case, the isolated nucleic acids can be single- or double-stranded. Typically, the isolated nucleic acids having at least one strand with both sense and antisense sequences that are complementary to each other also contain one or more cis-acting ribozyme sequences on the same strand containing the sense and antisense sequences. For example, a single-stranded RNA molecule can contain a sense sequence followed by an antisense sequence followed by a cis-acting ribozyme sequence. Likewise, the isolated nucleic acids having at least one strand that is a template for both sense and antisense sequences can be configured such that the strand that is a template for the sense and antisense sequences is also a template for one or more cis-acting ribozyme sequences. For example, a double-stranded DNA molecule can have one strand that is a template for synthesis of a single-stranded RNA molecule having a sense sequence followed by an antisense sequence followed by a cis-acting ribozyme sequence.

In one embodiment, isolated nucleic acids can be designed to have at least one strand with (1) sense and antisense sequences that are complementary to each other and (2) one or more cis-acting ribozyme sequences. The cis-acting ribozyme sequences can be positioned anywhere along the strand. For example, a cis-acting ribozyme sequence can be positioned 3′ of the sense and antisense sequences. In this case, the cis-acting ribozyme sequence can be used to limit the number of nucleotides 3′ of the sense and antisense sequences. In another example, cis-acting ribozyme sequences can be positioned 5′ and 3′ of the sense and antisense sequences as well as between the sense and antisense sequences to form the following 5′ to 3′ configuration: a first cis-acting ribozyme sequence followed by a sense sequence followed by a second cis-acting ribozyme sequence followed by an antisense sequence followed by a third cis-acting ribozyme sequence. In this case, the cis-acting ribozyme sequences can be used to liberate the sense and antisense sequences, which can then form double-stranded RNA molecules capable of acting as siRNAs.

In another embodiment, isolated nucleic acids can be designed to have at least one strand that is a template for (1) sense and antisense sequences that are complementary to each other and (2) one or more cis-acting ribozyme sequences. In this embodiment, the isolated nucleic acids can be designed such that the template strand is transcribed to form a single RNA molecule containing the sense and antisense sequences and the one or more cis-acting ribozyme sequences. The cis-acting ribozyme sequences can be positioned anywhere along the RNA molecule. For example, a cis-acting ribozyme sequence can be positioned 3′ of the sense and antisense sequences. In this case, the cis-acting ribozyme sequence can be used to limit the number of nucleotides 3′ of the sense and antisense sequences. In another example, cis-acting ribozyme sequences can be positioned 5′ and 3′ of the sense and antisense sequences as well as between the sense and antisense sequences to form the following 5′ to 3′ configuration: a first cis-acting ribozyme sequence followed by a sense sequence followed by a second cis-acting ribozyme sequence followed by an antisense sequence followed by a third cis-acting ribozyme sequence. In this case, the cis-acting ribozyme sequences can be used to liberate the sense and antisense sequence, which can then form double-stranded RNA molecules capable of acting as siRNAs.

The isolated nucleic acids provided herein can be used to form RNA molecules having the ability to induce RNA interference. For example, isolated nucleic acids with one strand containing sense, antisense, and cis-acting ribozyme sequences can form (1) double-stranded RNA and/or (2) single-stranded RNA upon cleavage by one or more cis-acting ribozyme sequences. Likewise, isolated nucleic acids having at least one strand that is a template for an RNA molecule containing sense, antisense, and cis-acting ribozyme sequences can form (1) double-stranded RNA and/or (2) single-stranded RNA upon cleavage of the RNA molecule by one or more cis-acting ribozyme sequences. In each case, the double-stranded RNA can result from the bonding interaction between the sense and antisense sequences. Typically, the double-stranded RNA is about 15-35 nucleotides in length and functions as siRNA molecules. The single-stranded RNA in each case can have zero, one, two, three, four, five, or more hairpin loop structures. In some embodiments, the single-stranded RNA can have no more than one hairpin loop structure. These hairpin loop structures can result from the bonding interaction between the sense and antisense sequences of the single-stranded RNA. Typically, the stem portion of the hairpin loop structure is about 15-35 nucleotides in length and can induce RNA interference. In addition, the double- and single-stranded RNA can be enzymatically inactive (e.g., lack trans-acting ribozyme activity). For example, a single-stranded RNA having no more than one hairpin loop structure can lack trans-acting ribozyme activity.

In addition, the isolated nucleic acids provided herein can be designed such that the presence of RNA molecules having the ability to induce RNA interference is controlled in a temporal, inducible, or tissue-specific manner. For example, a DNA molecule having one strand that is a template for (1) sense and antisense sequences that are complementary to each other and (2) one or more cis-acting ribozyme sequences can be constructed to have a tissue-specific promoter that promotes transcription of the template strand in a particular cell type. In this case, the isolated nucleic acid can be used to form RNA molecules containing the sense and antisense sequences and the one or more cis-acting ribozyme sequences in those particular cells. Once the RNA molecules are formed, the cis-acting ribozyme sequences can cleave the RNA molecules, for example, to liberate the sense and antisense sequences from other nucleotide sequences of the originally transcribed RNA molecules. For example, cis-acting ribozyme sequences can be positioned such that sequences flanking the sense sequence and sequences flanking the antisense sequence are removed. In this case, the RNA molecules containing the sense sequence can bind to the RNA molecules containing the complementary antisense sequence to form siRNA molecules.

Controlling the presence of RNA molecules having the ability to induce RNA interference in a temporal, inducible, or tissue-specific manner using promoter sequences can provide scientists with powerful tools for down-regulating mRNAs at particular stages of development or in particular cell types. These tools have several advantages over transfecting cells with (1) double-stranded RNA and (2) DNA constructs lacking the ability to transcribe cis-acting ribozyme sequences. First, while transfected double-stranded RNA molecules can be used as substrates for degradation by endonuclease RNase III to yield siRNA molecules, the RNA is labile and subject to degradation by a number of other nucleases. In addition, the delivery of double-stranded RNA molecules relies on the efficiency of transfection of the double-stranded RNA at a particular tissue or cell type. Second, the use of DNA constructs containing tissue-specific promoters that are promoters for RNA polymerase II can lead to transcription termination problems, resulting in lengthy RNA transcripts not suitable for subsequent siRNA generation. Using DNA constructs containing promoters for RNA polymerase III (e.g., U2, H1, or U6 promoters) can solve transcription termination problems, however, promoters for RNA polymerase III typically do not control transcription in a temporal, inducible, or tissue-specific manner. Transcription by RNA polymerase III is usually ubiquitous. In addition, RNA transcripts resulting from transcription by RNA polymerase III often remain nucleoplasmic.

The invention also provides cells, viruses, and transgenic animals (e.g., transgenic non-human animals) containing one or more of the isolated nucleic acids provided herein as well as methods for using one or more of the isolated nucleic acids provided herein to reduce the level of an RNA (e.g., an mRNA) within a cell. For example, an isolated nucleic acid provided herein can be used to form RNA molecules having the ability to induce RNA interference in that they can target an RNA sequence such as an mRNA from a virus, bacteria, fungus, parasite, plant, or animal (e.g., a mammal such as a human).

Cells containing one or more of the isolated nucleic acids provided herein can be used to generate substantially pure preparations of the isolated nucleic acid. For example, E. coli cells containing an isolated nucleic acid can be grown such that large amounts of the isolated nucleic acid are produced. In addition, cells containing the isolated nucleic acids provided herein can provide scientists with tools for studying gene function. For example, mammalian cells can be designed to contain an isolated nucleic acid provided herein such that siRNA molecules targeting a mammalian mRNA are formed within those cells. The phenotype of these resulting cells can be studied to determine the particular function of the targeted mammalian mRNA.

Viruses containing one or more of the isolated nucleic acids provided herein can be used to deliver isolated nucleic acids to cells. For example, neuron-specific viruses (e.g., herpes viruses) can be designed to contain an isolated nucleic acid provided herein such that that isolated nucleic acid can be delivered to neuronal cells. The viruses can contain additional modifications. For example, viruses can be designed to infect specific cells or groups of cells not normally infected by those viruses. See, e.g., Fields Virology, 3rd Edition (Fields, B., Knipe, D., and Howley, P, eds.), Lippincott Williams & Wilkins, New York, N.Y., 1996, pg. 186-191. In addition, viruses can be designed to lack virulence. For example, replication-defective viruses can be designed to contain an isolated nucleic acid provided herein.

Transgenic animals containing one or more of the isolated nucleic acids provided herein can be used to make knockout animals. For example, an isolated nucleic acid provided herein can be introduced into the genome of an animal such that each cell of the animal produces siRNA molecules that target a particular mRNA, thereby reducing or eliminating the presence of that particular mRNA within cells. Using the isolated nucleic acids provided herein to make knockout animals can avoid the need for homologous recombination. For example, knockout animals can be produced using standard transgenic technology without a homologous recombination step since disruption of the endogenous targeted gene sequences is not needed. As described herein, the isolated nucleic acids can contain promoter sequences such that RNA molecules having the ability to induce RNA interference are formed in specific cell types. Thus, transgenic animals can be produced such that each cell of the animal contains the isolated nucleic acid but only specific cells transcribe the isolated nucleic acid. In this case, the specific cells can form RNA molecules having the ability to induce RNA interference, while cells other than those specific cells do not.

In general, one aspect of the invention features an isolated nucleic acid containing a strand that is a template for an RNA molecule that contains a sense nucleic acid sequence, an antisense nucleic acid sequence, and a cis-acting ribozyme sequence. The sense nucleic acid sequence is complementary to the antisense nucleic acid sequence, and the sense and antisense nucleic acid sequences form double-stranded RNA upon cleavage of the RNA molecule by the cis-acting ribozyme sequence. The antisense nucleic acid sequence can be complementary to a viral mRNA sequence. The antisense nucleic acid sequence can be complementary to a mammalian mRNA sequence. The sense nucleic acid sequence can be at least 15 nucleotides in length. The sense nucleic acid sequence can be from about 15 to about 300 nucleotides in length. The sense nucleic acid sequence can be from about 15 to about 50 nucleotides in length. The sense nucleic acid sequence can contain the sequence as set forth in SEQ ID NO: 1, 2, 3, 4, 5, 6, 43, 44, 45, 46, 47, 48, 49, 50, 51, 52, 53, or 54. The cis-acting ribozyme sequence can be 3′ of the sense nucleic acid sequence or the antisense nucleic acid sequence. The cis-acting ribozyme sequence can be 3′ of the sense nucleic acid sequence and the antisense nucleic acid sequence. The cis-acting ribozyme sequence is 5′ of the sense nucleic acid sequence or the antisense nucleic acid sequence. The cis-acting ribozyme sequence can be 5′ of the sense nucleic acid sequence and the antisense nucleic acid sequence. The cis-acting ribozyme sequence can be between the sense nucleic acid sequence and the antisense nucleic acid sequence. The nucleic acid can be double stranded or single stranded. The nucleic acid can be DNA or RNA. The nucleic acid can contain a restriction site. The nucleic acid can be a plasmid. The nucleic acid can contain a promoter sequence that promotes transcription of the RNA molecule. The promoter sequence can be a tissue-specific promoter, cell-specific promoter, or pathogen-specific promoter. The promoter sequence can promote transcription by RNA polymerase II. The promoter sequence can promote transcription by RNA polymerase III. The promoter sequence can be an H1 promoter sequence or a U6 promoter sequence. The promoter sequence can be a mouse albumin enhancer promoter sequence, a transferrin promoter sequence, a probasin promoter sequence, or a whey acidic protein promoter sequence. The promoter sequence can be a keratin 7 promoter sequence, a keratin 13 promoter sequence, or a keratin enhancer promoter sequence. The RNA molecule can be transcribed from the nucleic acid when the nucleic acid is within a cell. The cell can be selected from the group consisting of kidney cells, skin cells (e.g., keratinocytes), liver cells, neurons, muscle cells, and lymphocytes. The strand can be a template for more than one cis-acting ribozyme sequence. Each of the more than one cis-acting ribozyme sequence can be different. One of the more than one cis-acting ribozyme sequence can be 5′ of the sense nucleic acid sequence and the antisense nucleic acid sequence. Another of the more than one cis-acting ribozyme sequence can be 3′ of the sense nucleic acid sequence and the antisense nucleic acid sequence. The sense nucleic acid sequence and the antisense nucleic acid sequence can each be flanked by at least one of the more than one cis-acting ribozyme sequence. The strand can be a template for three cis-acting ribozyme sequences.

In another embodiment, the invention features an isolated nucleic acid containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA molecule by the cis-acting ribozyme sequence, and where the single-stranded RNA contains no more than one hairpin loop structure and lacks RNA cleaving activity. The cis-acting ribozyme sequence can be 3′ of the sense nucleic acid sequence and the antisense nucleic acid sequence. The cis-acting ribozyme sequence can be 5′ of the sense nucleic acid sequence and the antisense nucleic acid sequence. The nucleic acid can be double stranded or single stranded. The nucleic acid can be DNA or RNA. The nucleic acid can contain a promoter sequence that promotes transcription of the RNA molecule. The nucleic acid can contain a restriction site. The nucleic acid can be a plasmid. The nucleic acid can contain a promoter sequence that promotes transcription of the RNA molecule. The promoter sequence can be a tissue-specific promoter, cell-specific promoter, or pathogen-specific promoter. The promoter sequence can promote transcription by RNA polymerase II. The promoter sequence can promote transcription by RNA polymerase III. The promoter sequence can be an H1 promoter sequence or a U6 promoter sequence. The promoter sequence can be a mouse albumin enhancer promoter sequence, a transferrin promoter sequence, a probasin promoter sequence, or a whey acidic protein promoter sequence. The promoter sequence can be a keratin 7 promoter sequence, a keratin 13 promoter sequence, or a keratin enhancer promoter sequence. The RNA molecule can be transcribed from the nucleic acid when the nucleic acid is within a cell. The cell can be selected from the group consisting of kidney cells, skin cells (e.g., keratinocytes), liver cells, neurons, muscle cells, and lymphocytes. The strand can be a template for more than one cis-acting ribozyme sequence. Each of the more than one cis-acting ribozyme sequence can be different. One of the more than one cis-acting ribozyme sequence can be 5′ of the sense nucleic acid sequence and the antisense nucleic acid sequence, and another of the more than one cis-acting ribozyme sequence can be 3′ of the sense nucleic acid sequence and the antisense nucleic acid sequence.

Another embodiment of the invention features an isolated nucleic acid containing an RNA strand that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA strand by the cis-acting ribozyme sequence. The nucleic acid can be single stranded. The RNA strand can contain more than one cis-acting ribozyme sequence. Each of the more than one cis-acting ribozyme sequence can be different. The sense nucleic acid sequence and the antisense nucleic acid sequence can each be flanked by at least one of the more than one cis-acting ribozyme sequence.

Another embodiment of the invention features an isolated nucleic acid containing an RNA strand that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA strand by the cis-acting ribozyme sequence, and where the single-stranded RNA contains no more than one hairpin loop structure and lacks RNA cleaving activity. The nucleic acid can be single stranded. The RNA strand can contain more than one cis-acting ribozyme sequence. Each of the more than one cis-acting ribozyme sequence can be different. One of the more than one cis-acting ribozyme sequence can be 5′ of the sense nucleic acid sequence and the antisense nucleic acid sequence, and another of the more than one cis-acting ribozyme sequence can be 3′ of the sense nucleic acid sequence and the antisense nucleic acid sequence.

Another embodiment of the invention features a composition containing a pharmaceutically acceptable carrier and an isolated nucleic acid selected from the group consisting of: (a) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA molecule by the cis-acting ribozyme sequence, (b) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA molecule by the cis-acting ribozyme sequence, and where the single-stranded RNA contains no more than one hairpin loop structure and lacks RNA cleaving activity, (c) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA strand by the cis-acting ribozyme sequence, and (d) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA strand by the cis-acting ribozyme sequence, and where the single-stranded RNA contains no more than one hairpin loop structure and lacks RNA cleaving activity.

In another aspect, the invention features an isolated cell containing an isolated nucleic acid selected from the group consisting of: (a) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA molecule by the cis-acting ribozyme sequence, (b) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA molecule by the cis-acting ribozyme sequence, and where the single-stranded RNA contains no more than one hairpin loop structure and lacks RNA cleaving activity, (c) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA strand by the cis-acting ribozyme sequence, and (d) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA strand by the cis-acting ribozyme sequence, and where the single-stranded RNA contains no more than one hairpin loop structure and lacks RNA cleaving activity.

Another aspect of the invention features a virus containing an isolated nucleic acid selected from the group consisting of: (a) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA molecule by the cis-acting ribozyme sequence, (b) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA molecule by the cis-acting ribozyme sequence, and where the single-stranded RNA contains no more than one hairpin loop structure and lacks RNA cleaving activity, (c) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA strand by the cis-acting ribozyme sequence, and (d) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA strand by the cis-acting ribozyme sequence, and where the single-stranded RNA contains no more than one hairpin loop structure and lacks RNA cleaving activity. The virus can be a retrovirus, adenovirus, herpes virus, adeno-associated viruse, lentivirus, baculovirus, cauliflower mosaic virus, tobacco mosaic virus, togavirus, poliovirus, cytomegalovirus, Paramyxovirus, Epstein-Barr virus, human papillomavirus, or hepatitis C virus.

Another aspect of the invention features a nonhuman transgenic animal, where the genome of the nonhuman transgenic animal contains a nucleic acid sequence, present on one strand, that is a template for an RNA molecule that contains: (a) a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA molecule by the cis-acting ribozyme sequence, or (b) a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA molecule by the cis-acting ribozyme sequence, and where the single-stranded RNA contains no more than one hairpin loop structure and lacks RNA cleaving activity. The nonhuman transgenic animal can be a mouse.

Another aspect of the invention features a method of identifying sequences capable of inducing RNA interference against a target mRNA, the method including: (a) introducing a vector preparation into cells, where each vector of the vector preparation contains: (1) a target nucleic acid sequence, where the target nucleic acid sequence is a template for the target mRNA; (2) a reporter nucleic acid sequence, where the reporter nucleic acid sequence encodes a polypeptide, and where the target nucleic acid sequence and the reporter nucleic acid sequence are transcribed as a single fusion mRNA; and (3) a promoter sequence region, where the promoter sequence region contains: (i) a member of a plurality of test nucleic acid sequences, and (ii) two promoter sequences operably linked to the member in an arrangement that promotes transcription of both strands of the member; (b) identifying at least one cell lacking the polypeptide; and (c) obtaining the sequence of the member from the cell identified in step (b), thereby identifying the sequence as being capable of inducing RNA interference against the target mRNA. The polypeptide can be a fluorescent polypeptide or can be lethal to the cell.

Another aspect of the invention features a method for reducing the level of an mRNA in a cell, the method including introducing an isolated nucleic acid into the cell, where the isolated nucleic acid is selected from the group consisting of: (a) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA molecule by the cis-acting ribozyme sequence, (b) isolated nucleic acids containing a strand that is a template for an RNA molecule that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA molecule by the cis-acting ribozyme sequence, and where the single-stranded RNA contains no more than one hairpin loop structure and lacks RNA cleaving activity, (c) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, and where the sense and antisense nucleic acid sequences form double-stranded RNA upon cleavage of the RNA strand by the cis-acting ribozyme sequence, and (d) isolated nucleic acids containing an RNA strand that contains a sense nucleic acid sequence, an antisense nucleic acid sequence, and a cis-acting ribozyme sequence, where the sense nucleic acid sequence is complementary to the antisense nucleic acid sequence, where the sense and antisense nucleic acid sequences form a single-stranded RNA upon cleavage of the RNA strand by the cis-acting ribozyme sequence, and where the single-stranded RNA contains no more than one hairpin loop structure and lacks RNA cleaving activity, where the presence of the isolated nucleic acid within the cell reduces the level of the mRNA in the cell.

Another aspect of the invention features a mixture containing at least two isolated nucleic acids, where one of the at least two isolated nucleic acids contains a strand that is a template for an RNA molecule containing a sense nucleic acid sequence and a first cis-acting ribozyme sequence, where another of the at least two isolated nucleic acids contains a strand that is a template for an RNA molecule containing an antisense nucleic acid sequence and a second cis-acting ribozyme sequence, and where the sense nucleic acid sequence is complementary to the antisense nucleic acid sequence.

Another aspect of the invention features an isolated nucleic acid containing: (a) a target nucleic acid sequence, wherein the target nucleic acid sequence is a template for target mRNA, and (b) a promoter sequence region, wherein the promoter sequence region contains (i) a nucleic acid sequence and (ii) two promoter sequences operably linked to the nucleic acid sequence in an arrangement that promotes transcription of both strands of the nucleic acid sequence, wherein the nucleic acid sequence contains a sequence present in the target nucleic acid sequence, and wherein transcription, within a cell, of the target nucleic acid sequence and both strands of the nucleic acid sequence is capable of inducing RNA interference against the target mRNA. The target nucleic acid sequence can be a viral sequence. The target nucleic acid sequence can be an HPV sequence or an HBV sequence. One of the two promoter sequences can be a U6 promoter sequence or an H1 promoter sequence. The two promoter sequences can be the same or different. The two promoter sequences can be separated by no more than 200 base pairs, no more than 100 base pairs, or no more than 50 base pairs. The promoter sequence region can contain more than two promoter sequences (e.g., three, four, five, six, seven, or more promoter sequences). The sequence present in the target nucleic acid sequence can be between 15 and 50 nucleotides in length. The sequence present in the target nucleic acid sequence can be between 18 and 25 nucleotides in length. The isolated nucleic acid can contain a reporter nucleic acid sequence, wherein the reporter nucleic acid sequence encodes a polypeptide, and wherein the target nucleic acid sequence and the reporter nucleic acid sequence are transcribed as a single fusion mRNA. The polypeptide can be a fluorescent polypeptide. The polypeptide can be a green fluorescent polypeptide (e.g., GFP).

Another aspect of the invention features a nucleic acid library containing isolated nucleic acids, wherein each isolated nucleic acid contains: (a) a target nucleic acid sequence, wherein the target nucleic acid sequence is a template for target mRNA, and (b) a promoter sequence region, wherein the promoter sequence region contains (i) a nucleic acid sequence and (ii) two promoter sequences operably linked to the nucleic acid sequence in an arrangement that promotes transcription of both strands of the nucleic acid sequence, wherein the nucleic acid sequence is different for each isolated nucleic acid, wherein the nucleic acid sequence of at least one of the isolated nucleic acids contains a sequence present in the target nucleic acid sequence, and wherein transcription, within a cell, of the target nucleic acid sequence and both strands of the nucleic acid sequence of at least one of the isolated nucleic acids is capable of inducing RNA interference against the target mRNA. The target nucleic acid sequence can be a viral sequence. The target nucleic acid sequence can be an HPV sequence or an HBV sequence. One of the two promoter sequences can be a U6 promoter sequence or an H1 promoter sequence. The two promoter sequences can be the same or different. The two promoter sequences can be separated by no more than 200 base pairs, no more than 100 base pairs, or no more than 50 base pairs. The promoter sequence region can contain more than two promoter sequences. The sequence present in the target nucleic acid sequence can be between 15 and 50 nucleotides in length. The sequence present in the target nucleic acid sequence can be between 18 and 25 nucleotides in length. The isolated nucleic acid can contain a reporter nucleic acid sequence, wherein the reporter nucleic acid sequence encodes a polypeptide, and wherein the target nucleic acid sequence and the reporter nucleic acid sequence are transcribed as a single fusion mRNA. The polypeptide can be a fluorescent polypeptide. The polypeptide can be a green fluorescent polypeptide (e.g., GFP).

Another aspect of the invention features an isolated nucleic acid containing: (a) a target nucleic acid sequence, wherein the target nucleic acid sequence is a template for target mRNA, and (b) a promoter sequence region, wherein the promoter sequence region contains a promoter sequence operably linked to a nucleic acid sequence, wherein one strand of the nucleic acid sequence is a template for a sense nucleic acid sequence and an antisense nucleic acid sequence, wherein the sense nucleic acid sequence is complementary to the antisense nucleic acid sequence, wherein the sense nucleic acid sequence contains a sequence present in the target mRNA, and wherein transcription, within a cell, of the target nucleic acid sequence and at least one strand of the nucleic acid sequence is capable of inducing RNA interference against the target mRNA. The target nucleic acid sequence can be a viral sequence. The target nucleic acid sequence can be an HPV sequence or an HBV sequence. The promoter sequence can be a U6 promoter sequence or an H1 promoter sequence. The promoter sequence region can contain two promoter sequences operably linked to the nucleic acid sequence in an arrangement that promotes transcription of both strands of the nucleic acid sequence. The two promoter sequences can be the same or different. The two promoter sequences can be separated by no more than 200 base pairs, no more than 100 base pairs, or no more than 50 base pairs. The promoter sequence region can contain more than two promoter sequences. The sequence present in the target nucleic acid sequence can be between 15 and 50 nucleotides in length. The sequence present in the target nucleic acid sequence can be between 18 and 25 nucleotides in length. The transcription product from at least one strand of the nucleic acid sequence can be capable of forming a hairpin loop structure. At least a portion of the stem portion of the hairpin loop structure can be formed by the sense nucleic acid sequence and the antisense nucleic acid sequence. The isolated nucleic acid can contain a reporter nucleic acid sequence, wherein the reporter nucleic acid sequence encodes a polypeptide, and wherein the target nucleic acid sequence and the reporter nucleic acid sequence are transcribed as a single fusion mRNA. The polypeptide can be a fluorescent polypeptide. The polypeptide can be a green fluorescent polypeptide.

Another aspect of the invention features a nucleic acid library containing isolated nucleic acids, wherein each isolated nucleic acid contains a promoter sequence operably linked to a nucleic acid sequence, wherein one strand of the nucleic acid sequence is a template for a sense nucleic acid sequence and an antisense nucleic acid sequence, wherein the sense nucleic acid sequence is complementary to the antisense nucleic acid sequence, wherein the sense nucleic acid sequence is different for each isolated nucleic acid, and wherein transcription, within a cell, of a target nucleic acid sequence and at least one strand of the nucleic acid sequence of at least one of the isolated nucleic acids is capable of inducing RNA interference against a target mRNA, the target nucleic acid sequence being a template for the target mRNA. Each isolated nucleic acid can contain the target nucleic acid sequence. The sense nucleic acid sequence of at least one of the isolated nucleic acids can contain a sequence present in the target mRNA. The target nucleic acid sequence can be a viral sequence. The target nucleic acid sequence can be an HPV sequence or an HBV sequence. The promoter sequence can be a U6 promoter sequence or an H1 promoter sequence. The isolated nucleic acids can contain two promoter sequences operably linked to the nucleic acid sequence in an arrangement that promotes transcription of both strands of the nucleic acid sequence. The two promoter sequences can be the same or different. The two promoter sequences can be separated by no more than 200 base pairs, no more than 100 base pairs, or no more than 50 base pairs. The isolated nucleic acids can contain more than two promoter sequences. The sequence present in the target nucleic acid sequence can be between 15 and 50 nucleotides in length. The sequence present in the target nucleic acid sequence can be between 18 and 25 nucleotides in length. The isolated nucleic acids can contain a reporter nucleic acid sequence, wherein the reporter nucleic acid sequence encodes a polypeptide, and wherein the target nucleic acid sequence and the reporter nucleic acid sequence are transcribed as a single fusion mRNA. The polypeptide can be a fluorescent polypeptide. The polypeptide can be a green fluorescent polypeptide (e.g., GFP).

Another aspect of the invention features a method for making a library containing isolated nucleic acids, wherein each isolated nucleic acid contains a nucleic acid sequence, wherein one strand of the nucleic acid sequence is a template for a sense nucleic acid sequence and an antisense nucleic acid sequence, wherein the sense nucleic acid sequence is complementary to the antisense nucleic acid sequence, wherein the sense nucleic acid sequence is different for each isolated nucleic acid, wherein transcription, within a cell, of a target nucleic acid sequence and at least one strand of the nucleic acid sequence of at least one of the isolated nucleic acids is capable of inducing RNA interference against a target mRNA, and wherein the target nucleic acid sequence is a template for the target mRNA, the method including: (a) obtaining a nucleic acid collection containing nucleic acid molecules, wherein one strand of each nucleic acid molecule contains the sense nucleic acid sequence or the antisense nucleic acid sequence, wherein the sense nucleic acid sequence or the antisense nucleic acid sequence is different for each nucleic acid molecule, wherein the one strand of each nucleic acid molecule contains a first sequence and a second sequence, wherein the first sequence is complementary to the second sequence, and wherein the first and second sequences are located 3′ of the sense nucleic acid sequence or the antisense nucleic acid sequence of each nucleic acid molecule, and (b) amplifying the nucleic acid collection in an amplification reaction under conditions wherein the 3′ end of each nucleic acid molecule is extended using a portion of the 5′ end of each nucleic acid molecule as a template to form an extended nucleic acid collection containing extended nucleic acid molecules, wherein the amplification reaction amplifies the extended nucleic acid molecules, wherein one strand of each extended nucleic acid molecule contains the sense nucleic acid sequence and the antisense nucleic acid sequence, and wherein the extended nucleic acid collection is the library. The method can include removing a portion of the sequence located between the sense nucleic acid sequence and the antisense nucleic acid sequence of each extended nucleic acid molecule. After the removing step, the sense nucleic acid sequence and the antisense nucleic acid sequence of each extended nucleic acid molecule can be separated by 4 to 20 nucleotides. The method can include inserting each extended nucleic acid molecule into an expression vector.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic diagram of a single-stranded RNA molecule that can be transcribed from p1CLIP_{S}/HPV16_{C+50-68+GUU}. The sense sequence is 5′-CCAGAAAGUUACCACAGUU-3′ (SEQ ID NO: 1, a HPV16₅₀₋₆₈ sequence) and is positioned between two cis-acting ribozyme cleavage sites designated S1 and S2. This sense sequence is from the HPV sequence set forth in GenBank Accession No. gi333031. The sense sequence can be designed to correspond to HPV sequences from other HPV strains such as the Hershey strain. For example, the sense sequence can be 5′-CCGGAAAGUUA-CCACAGUU-3′ (SEQ ID NO: 2, a HPV16_{50-68(H)} sequence). Such sense sequences can be from the HPV sequences set forth in GenBank Accession Nos. gi3377787, 2196720, 1098775, and 1098731.

FIG. 2 is a schematic diagram of a single-stranded RNA molecule that can be transcribed from p1CLIP_{AS}/HPV16_{C+68-50+GUU}. The antisense sequence is 5′-AACUGU-GGUAACUUUCUGG-3′ (SEQ ID NO: 3, the HPV16₆₈₋₅₀ sequence) and is positioned between two cis-acting ribozyme cleavage sites designated S1 and S2. This antisense sequence is from the HPV sequence set forth in GenBank Accession No. gi333031. The antisense sequence can be designed to correspond to HPV sequences from other HPV strains such as the Hershey strain. For example, the antisense sequence can be 5′-AACUGU-GGUAACUUUCCGG-3′ (SEQ ID NO: 4, a HPV16_{68-50(H)} sequence). Such antisense sequences can be from the HPV sequences set forth in GenBank Accession Nos. gi3377787, 2196720, 1098775, and 1098731.

FIG. 3 is a schematic diagram of a single-stranded RNA molecule that can be transcribed from p1CLIP_{S}/HPV16_{57-77+UU}. The sense sequence is 5′-GUUACCA-CAGUUAUGCACAGA-3′ (SEQ ID NO: 5, the HPV16₅₇₋₇₇ sequence) and is positioned between two cis-acting ribozyme cleavage sites designated S1 and S2.

FIG. 4 is a schematic diagram of a single-stranded RNA molecule that can be transcribed from p1CLIP_{AS}/HPV16_{77-57+UU}. The sense sequence is 5′-UCUGU-GCAUAACUGUGGUAAC-3′ (SEQ ID NO: 6, the HPV16₇₇₋₅₇ sequence) and is positioned between two cis-acting ribozyme cleavage sites designated S1 and S2.

FIG. 5 is a schematic diagram of a single-stranded RNA molecule that can be transcribed from p2CLIP_{S}/HPV16_{C+50-68+GUU}.

FIG. 6 is a schematic diagram of a single-stranded RNA molecule that can be transcribed from p2CLIP_{AS}/HPV16_{C+68-50+GUU}.

FIG. 7 is a schematic diagram of a single-stranded RNA molecule that can be transcribed from p1CHOP_{S}/HPV16_{C+50-68+GUU}. The sense sequence is positioned between two cis-acting ribozyme cleavage sites designated S1 and S2.

FIG. 8 is a schematic diagram of a single-stranded RNA molecule that can be transcribed from p1CHOP_{AS}/HPV16_{C+68-50+GUU}. The antisense sequence is positioned between two cis-acting ribozyme cleavage sites designated S1 and S2.

FIG. 9 is a schematic diagram of a single-stranded RNA molecule that can be transcribed from p2CHOP_{S}/HPV16_{C+50-68+GUU}. The sense sequence is positioned between two cis-acting ribozyme cleavage sites designated S1 and S2.

FIG. 10 is a schematic diagram of a single-stranded RNA molecule that can be transcribed from p2CHOP_{AS}/HPV16_{C+68-50+GUU}. The antisense sequence is positioned between two cis-acting ribozyme cleavage sites designated S1 and S2.

FIG. 11 is a schematic diagram of an RNA transcribed from a pSIR cassette containing any two (or more) of the following cassettes with one containing a sense sequence and another containing an antisense sequence: (1) p1CLIP, (2) p2CLIP, (3) p1CHOP, and (4) p2CHOP. The four inverted-U symbols represent loop structures, while the four bulged hairpin loop structures represent cis-acting ribozymes. S1, S2, S3, and S4 identify cis-acting ribozyme cleavage sites.

FIG. 12 is a listing of the sense and antisense sequences that can be self liberated from an RNA molecule transcribed from a pSIR cassette containing p1CLIP, p1CHOP, or a combination of p1CLIP and p1CHOP cassettes.

FIG. 13 is a listing of the sense and antisense sequences that can be self liberated from an RNA molecule transcribed from a pSIR cassette containing p2CLIP, p2CHOP, or a combination of p2CLIP and p2CHOP cassettes.

FIG. 14 is a schematic diagram of an RNA molecule with three cis-acting ribozyme sequences. This RNA molecule can be transcribed from a p2CLIP_{HR} cassette such as p2CLIP_{HR}/HPV16₄₇₋₆₈. S1, S2, and S3 identify cis-acting ribozyme cleavage sites.

FIG. 15 is a schematic diagram of an RNA molecule with three cis-acting ribozyme sequences. This RNA molecule can be transcribed from a p2CHOP_{HR} cassette such as P2CHOP_{HR}/HPV16₄₇₋₆₈. S1, S2, and S3 identify cis-acting ribozyme cleavage sites.

FIG. 16 is a listing of three sets of sense and antisense RNA sequences that can be transcribed from inserts placed into p1CLIP, p2CLIP, p1CHOP, or p2CHOP cassettes to form p1CLIP_{HR}, p2CLIP_{HR}, p10CHOP_{HR}, or p2CHOP_{HR} cassettes.

FIG. 17 is a schematic diagram of an RNA molecule that is transcribed from a pSNIP cassette. The inverted-U symbols represent loop structures, while the bulged hairpin loop structures represent cis-acting ribozymes. S1, S2, S3, S4, S5, and S6 identify cis-acting ribozyme cleavage sites. A pSNIP cassette can contain any two or more of the following cassettes: (1) p1CLIP_{HR}, (2) p2CLIP_{HR}, (3) p1CHOP_{HR}, and (4) p2CHOP_{HR}.

FIG. 18 is a schematic diagram of a single-stranded RNA molecule that can be transcribed from p2CLIP_{HP}/HPV16_{C+50-68+GUU}. The sequence of the loop portion of the hairpin loop is 5′-GUUCAAGACCC-3′ (SEQ ID NO: 7) and is located between the HPV sense and antisense sequences that form the stem portion.

FIG. 19 is a listing of three sets of sense and antisense RNA sequences that can be transcribed from inserts placed into p1CLIP, p2CLIP, p1CHOP, or p2CHOP cassettes to form p1CLIP_{HP}, p2CLIP_{HP}, p1CHOP_{HP}, or p2CHOP_{HP} cassettes.

FIG. 20 is a schematic diagram of an in vivo selection process. While the figure shows a U6 promoter and an H1 promoter, both promoters can be U6 promoters. The inserts can be from any source. For example, the inserts can be the inserts obtained from the in vitro selection process depicted in FIG. 21. The RNA-processing enzyme DICER and the RNA-Induced Silencing Complex (RISC) can be present endogenously within Flp-In 293 cells.

FIG. 21 is a schematic diagram of an in vitro selection process.

FIG. 22 is a bar graph plotting the relative level of HPV16 mRNA in cells containing the following nucleic acid. Column 1: cells containing p1CLIP_{HP}/HPV16_{C+50-68+GUU}; column 2: cells containing pSIR (p1CLIP_{S}/HPV16_{C+50-68+GUU} and p1CLIP_{AS}/HPV16_{C+68-50+GUU}); column 3: cells containing p1CLIP_{S}/HPV16_{C+50-68+GUU}; column 4: cells containing p2CLIP_{HR}/HPV16₄₇₋₆₈; column 5: cells containing p2CHOP_{HR}/HPV16₄₇₋₆₈; column 6: cells containing pSIR (p1CLIP_{S}/HPV16_{57-77+GUU} and p1CLIP_{AS}/HPV16_{77-57+GUU}); and column 7: control cells.

FIG. 23 contains two bar graphs. The top bar graph plots the relative level of HPV16 mRNA in cells containing the following nucleic acid. In each case, target mRNA expression was under the control of a CMV promoter sequence. Column 1: cells containing p1CLIP_{S}/HPV16_{C+50-68(H)+GUU}; Column 2: cells containing p1CLIP_{AS}/HPV16_{C+68-50(H)+GUU}; Column 3: cells containing pSIR (p1CLIP_{S}/HPV16_{C+50-68(H)+GUU} and p1CLIP_{AS}/HPV16_{C+64-50(H)+GUU}); Column 4: cells containing p1CLIP_{HP}/HPV16_{C+50-68(H)+GUU}; Column 5: cells containing pSIR (p1CLIP_{HP}/HPV16_{C+50-68(H)+GUU} and p1CLIP_{HP}/HPV16_{C+50-68(H)+GUU}); Column 6: cells containing p2CLIP_{HP}/HPV16_{C+50-68(H)+GUU}; Column 7: cells containing p2CHOP_{HP}/HPV16_{C+50-68(H)+GUU;} Column 8: cells containing pSNIP (p2CLIP_{HP}/HPV16_{C+50-68(H)+GUU} and p2CHOP_{HP}/HPV16_{C+50-68(H)+GUU}); Column 9: cells containing p2CLIP_{HR}/HPV16₄₇₋₆₈; Column 10: cells containing p2CHOP_{HR}/HPV16₄₇₋₆₈; Column 11: cells containing pSNIP (p2CLIP_{HR}/HPV16₄₇₋₆₇ and p2CHOP_{HR}/HPV16₄₇₋₆₈); and Column 12: control cells. The bottom bar graph plots the relative level of HPV16 mRNA in cells containing the following nucleic acid. In each case, target mRNA expression was under the control of a U6 promoter sequence. Column 1: cells containing p1CLIP_{S}/HPV16_{C+50-68(H)GUU}; Column 2: cells containing p1CLIP_{AS}/HPV16_{C+68-50(H)+GUU}; Column 3: cells containing pSIR (p1CLIP_{S}/HPV16_{C+50-68(H)+GUU} and p1CLIP_{AS}/HPV16_{C+68-50(H)+GUU}); Column 4: cells containing p1CLIP_{HP}/HPV16_{C+50-68(H)+GUU}; Column 5: cells containing pSIR (p1CLIP_{HP}/HPV16_{C+50-68(H)GUU} and p1CLIP_{HP}/HPV16_{C+50-68(H)GUU}); Column 6: cells containing p2CLIP_{HR}/HPV16₄₇₋₆₈; Column 7: cells containing p2CHOP_{HR}/HPV16₄₇₋₆₈; Column 8: cells containing pSNIP (p2CLIP_{HR}/HPV16₄₇₋₆₈ and p2CHOP_{HR}/HPV16₄₇₋₆₈); Column 9: cells containing a cassette that liberates trans-acting ribozymes targeting the HPV16₅₀₋₆₈ region; and Column 10: control cells.

FIG. 24 is a schematic diagram of an in vitro selection process that can be used to obtain an enriched pool of sequences that interact with a target mRNA.

FIG. 25 is a schematic diagram of a cloning process that can be used to obtain a library of hairpin loop sequences. Each strand of the final double-stranded product can encode both a sense and antisense sequence (e.g., the E21 and e21 sequences). These E21 and e21 sequences of a single-stranded RNA molecule can form the stem portion of a hairpin loop. The loop portion can be encoded by the 5′-TTCTAGAA-3′ (SEQ ID NO: 55) sequence.

FIG. 26 is a schematic diagram of an in vivo selection process. While the figure shows a U6 promoter and an H1 promoter, both promoters can be U6 promoters. The inserts can be from any source. For example, the inserts can be the inserts obtained from the in vitro selection process depicted in FIG. 21 or can be the inserts obtained from the cloning process depicted in FIG. 25. The depicted inserts are from FIG. 25. The RNA-processing enzyme DICER and the RNA-Induced Silencing Complex (RISC) can be present endogenously within Flp-In 293 cells.

### DETAILED DESCRIPTION

The invention provides isolated nucleic acids having at least one strand with both sense and antisense sequences that are complementary to each other. The invention also provides isolated nucleic acids having at least one strand that is a template for both sense and antisense sequences that are complementary to each other. In addition, the invention provides cells, viruses, and transgenic animals (e.g., transgenic non-human animals) containing one or more of the isolated nucleic acids provided herein as well as methods for using one or more of the isolated nucleic acids provided herein to reduce the level of an RNA (e.g., an mRNA) within a cell.

Nucleic Acids

The invention provides isolated nucleic acids. The term “nucleic acid” as used herein encompasses both RNA and DNA, including cDNA, genomic DNA, and synthetic (e.g., chemically synthesized) DNA. The nucleic acid can be double-stranded or single-stranded. En addition, nucleic acid can be circular or linear. In some embodiments, the nucleic acid can be a plasmid. The nucleic acid can contain one or more restriction sites.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, lentivirus, adenovirus, or herpes virus), or the genomic DNA of a prokaryote or eukaryote.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

In one embodiment, the invention provides isolated nucleic acids having at least one strand with both sense and antisense sequences that are complementary to each other. In another embodiment, the invention provides isolated nucleic acids having at least one strand that is a template for both sense and antisense sequences that are complementary to each other. The term “complementary” as used herein with reference to two nucleic acid sequences (e.g., sense and antisense sequences) means the two nucleic acid sequences are 100 percent complementary. The sense and antisense sequences can be part of a larger nucleic acid molecule or be part of separate nucleic acid molecules having sequences that are not complementary. The sense and antisense sequences can be any length greater than 12 nucleotides (e.g., 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides (e.g., from about 18 nucleotides to about 28 nucleotides, or from about 21 nucleotides to about 25 nucleotides).

The terms “sense” and “antisense” as used herein with respect to two sequences refer to the two members of a pair of complementary sequences. If one member of the pair is designated a sense sequence, then the other member of the pair is designated an antisense sequence. The sense and antisense sequences of an isolated nucleic acid can have any sequence. For example, an antisense sequence can be (1) a sequence complementary to an mRNA sequence or (2) a sequence that is not complementary to an mRNA sequence. In each case, the sense sequence is the sequence complementary to the antisense sequence.

In some embodiments, an antisense sequence is a sequence complementary to an mRNA sequence of a virus (e.g., papilloma virus, hepatitis B virus, herpes virus, retrovirus, adenovirus, or HIV), parasite (Schlistosoma mansoni, Fasciola, or Paragonimus), bacterium (e.g., E. coli, Staphlococcus, Pseudomonas, Streptococcus, Nisseria, or Haemophilus), pathogenic protozoa (e.g., Entamoeba, Plasmodium, Tryphnosoma, or Toxoplasm), fungus (e.g., Aspergillus, Candida, Cryptococcus, or Coccidioides), plant (e.g., corn, wheat, or rice), or animal (e.g., a mammal such as a mouse, rat, pig, cow, monkey, or human). In these cases, the sense sequence complementary to the antisense sequence is a sequence present within the mRNA of a virus, parasite, bacterium, fungus, plant, or animal, respectively. Typically, sense and antisense sequences are designed to correspond to a 15-30 nucleotide sequence of a target mRNA such that the level of that target mRNA is reduced via RNA interference. Examples of sense and antisense sequences include, without limitation, those sequences set forth in Table 1.

TABLE 1
Sense and antisense sequences.

| | | |
|---|---|---|
| Name | Target | Sequence |

| | | |
|---|---|---|
| HPV16₅₀₋₆₈ | Human papillomavirus | 5′-CCAGAAAGUUACCACAGUU-3′ |
| | type 16 E6/E7 | SEQ ID NO: 1. |
| HPV16₆₈₋₅₀ | Human papillomavirus | 5′-AACUGUGGUAACUUUCUGG-3′ |
| | type 16 E6/E7 | SEQ ID NO: 3. |
| HPV16_{50-68(H)} | Human papillomavirus | 5′-CCGGAAAGUUACCACAGUU-3′ |
| | type 16 E6/E7 | SEQ ID NO: 2. |
| | (Hershey strain) | |
| HPV16_{68-50(H)} | Human papillomavirus | 5′-AACUGU-GGUAACUUUCCGG-3′ |
| | type 16 E6/E7 | SEQ ID NO: 4. |
| | (Hershey strain) | |
| HPV16₅₇₋₇₇ | Human papillomavirus | 5′-GUUACCACAGUUAUGCACAGA-3′ |
| | type 16 E6/E7 | SEQ ID NO: 5. |
| HPV16₇₇₋₅₇ | Human papillomavirus | 5′-UCUGUGCAUAACUGUGGUAAC-3′ |
| | type 16 E6/E7 | SEQ ID NO: 6. |
| HPV16₄₇₋₆₈ | Human papillomavirus | 5′-GACCCAGAAAGUUACCACAGUU-3′ |
| | type 16 E6/E7 | SEQ ID NO: 43 |
| HPV16₆₈₋₄₇ | Human papillomavirus | 5′-AACUGUGGUAACUUUCUGGGUC-3′ |
| | type 16 E6/E7 | SEQ ID NO:44 |
| HPV16_{47-68(H)} | Human papillomavirus | 5′-GACCCGGAAAGUUACCACAGUU-3′ |
| | type 16 E6/E7 | SEQ ID NO: 45 |
| | (Hershey strain) | |
| HPV16_{68-47(H)} | Human papillomavirus | 5′-AACUGUGGUAACUUUCCGGGUC-3′ |
| | type 16 E6/E7 | SEQ ID NO: 46 |
| | (Hershey strain) | |
| HPV16₅₅₋₇₄ | Human papillomavirus | 5′-AAGUUACCACAGUUAUGCAC-3′ |
| | type 16 E6/E7 | SEQ ID NO: 47 |
| HPV16₇₄₋₅₅ | Human papillomavirus | 5′-GUGCAUAACUGUGGUAACUUA-3′ |
| | type 16 E6/E7 | SEQ ID NO: 48 |
| HBV₈₅₉₋₈₈₁ | Human hepatitis B | 5′-GAAUUUGGAGCUACUGUGGAGUU-3′ |
| | virus strain ayw | SEQ ID NO: 49 |
| | variant precore/core | |
| HBV₈₈₁₋₈₅₉ | Human hepatitis B | 5′-AACUCCACAGUAGCUCCAAAUUC-3′ |
| | virus strain ayw | SEQ ID NO: 50 |
| | variant | |
| HBV₈₆₀₋₈₈₁ | Human hepatitis B | 5′-AAUUUGGAGCUACUGUGGAGUU-3′ |
| | virus strain ayw | SEQ ID NO: 51 |
| | variant precore/core | |
| HBV₈₈₁₋₈₆₀ | Human hepatitis B | 5′-AACUCCACAGUAGCUCCAAAUU-3′ |
| | virus strain ayw | SEQ ID NO: 52 |
| | variant precore/core | |
| HBV₈₆₂₋₈₈₂ | Human hepatitis B | 5′-UUUGGAGCUACUGUGGAGUUA-3′ |
| | virus strain ayw | SEQ ID NO: 53 |
| | variant precore/core | |
| HBV₈₈₂₋₈₆₂ | Human hepatitis B | 5′-UAACUCCACAGUAGCUCCAAA-3′ |
| | virus strain ayw | SEQ ID NO: 54 |
| | variant precore/core | |

In addition, sense and antisense sequences can be designed to target mRNA that encodes polypeptides necessary for the growth and/or survival of parasites, bacteria, viruses, fungi, and tumors. For example, sense and antisense sequences can be designed to target mRNA that encodes polypeptides essential for cell viability or fitness, DNA biosynthesis, cell division, transcription, reverse transcription, metabolism, catabolism, angiogenesis, and cellular respiration (e.g., Her-2/neu, Akt-3, and UGT2B7 polypeptides). Sense and antisense sequences also can be designed to target structural RNA molecules such as DD3.

The isolated nucleic acids provided herein can have (1) at least one strand with more than one sense sequence (e.g., 2, 3, 4, 5, 6, 7, 8, 9, 10 or more sense sequences) or (2) at least one strand that is a template for more than one sense sequence (e.g., 2, 3, 4, 5, 6, 7, 8, 9, 10 or more sense sequences). Likewise, the isolated nucleic acids provided herein can have (1) at least one strand with more than one antisense sequence (e.g., 2, 3, 4, 5, 6, 7, 8, 9, 10 or more antisense sequences) or (2) at least one strand that is a template for more than one antisense sequence (e.g., 2, 3, 4, 5, 6, 7, 8, 9, 10 or more antisense sequences). For example, an isolated nucleic acid can have at least one strand that is a template for two sense sequences and two antisense sequences. The multiple sense sequences can be identical or different, and the multiple antisense sequences can be identical or different. For example, an isolated nucleic acid can have one strand that is a template for (1) two identical sense sequences and (2) two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length.

The isolated nucleic acids provided herein can be designed to have any arrangement of sense and antisense sequences. For example, two identical sense sequence (1) can be followed by two identical antisense sequences or (2) can be positioned between two identical antisense sequences.

The isolated nucleic acids having at least one strand with both sense and antisense sequences that are complementary to each other can also have one or more cis-acting ribozyme sequences (e.g., 2, 3, 4, 5, 6, 7, 8, 9, 10, or more cis-acting ribozyme sequences) on the same strand having the sense and antisense sequences. For example, a single-stranded RNA molecule can contain a first cis-acting ribozyme sequence followed by a sense sequence followed by a second cis-acting ribozyme sequence followed by an antisense sequence followed by a third cis-acting ribozyme sequence. Likewise, the isolated nucleic acids having at least one strand that is a template for both sense and antisense sequences that are complementary to each other can be designed such that the strand that is a template for the sense and antisense sequences is also a template for one or more cis-acting ribozyme sequences (e.g., 2, 3, 4, 5, 6, 7, 8, 9, 10, or more cis-acting ribozyme sequences). For example, an isolated DNA molecule can contain one strand that is a template for a first cis-acting ribozyme sequence followed by a sense sequence followed by a second cis-acting ribozyme sequence followed by an antisense sequence followed by a third cis-acting ribozyme sequence.

In general, a ribozyme is a catalytic RNA molecule that cleaves RNA in a sequence specific manner. Ribozymes that cleave themselves are called cis-acting ribozymes, while ribozymes that cleave other RNA molecules are called trans-acting ribozymes. The term “cis-acting ribozyme sequence” as used herein refers to the sequence of an RNA molecule that has the ability to cleave the RNA molecule containing the cis-acting ribozyme sequence. A cis-acting ribozyme sequence can have any sequence provided it has the ability to cleave the RNA molecule containing the cis-acting ribozyme sequence. For example, a cis-acting ribozyme sequence can have a sequence from a hammerhead, axhead, or hairpin ribozyme. In addition, a cis-acting ribozyme sequence can have a sequence from a hammerhead, axhead, or hairpin ribozyme that is modified to have either slow cleavage activity or enhanced cleavage activity. For example, nucleotide substitutions can be made to modify cleavage activity as described elsewhere (see, e.g., Doudna and Cech, Nature, 418:222-228 (2002)). Examples of ribozyme sequences that can be used herein include, without limitation, those described herein as well as those described in U.S. Pat. No. 6,271,359, U.S. Pat. No. 5,824,519, and Doudna and Cech, Nature, 418:222-228 (2002).

When more than one cis-acting ribozyme sequence is used, the multiple cis-acting ribozyme sequences can be identical or different. For example, an isolated nucleic acid can have one strand that is a template for three cis-acting ribozyme sequences; where two are identical, and the third is different. The isolated nucleic acids provided herein can be designed to have any arrangement of sense, antisense, and cis-acting ribozyme sequences. For example, each sense and each antisense sequence can be flanked by at least one cis-acting ribozyme sequence. Other arrangements include, without limitation, the following: (1) a first cis-acting ribozyme followed by a sense sequence followed by a second cis-acting ribozyme followed by a third cis-acting ribozyme followed by an antisense sequence followed by a fourth cis-acting ribozyme, (2) a first cis-acting ribozyme followed by a sense sequence followed by an antisense sequence followed by a second cis-acting ribozyme, (3) a sense sequence followed by a first cis-acting ribozyme followed by a second cis-acting ribozyme followed by an antisense sequence followed by a third cis-acting ribozyme, (4) a sense sequence followed by a first cis-acting ribozyme followed by an antisense sequence followed by a second cis-acting ribozyme, (5) a first cis-acting ribozyme followed by a first sense sequence followed by a second cis-acting ribozyme followed by a third cis-acting ribozyme followed by a first antisense sequence complementary to the first sense sequence followed by a fourth cis-acting ribozyme followed by a fifth cis-acting ribozyme followed by a second sense sequence followed by a sixth cis-acting ribozyme followed by a seventh cis-acting ribozyme followed by a second antisense sequence complementary to the second sense sequence followed by an eighth cis-acting ribozyme, and (6) a first cis-acting ribozyme followed by a first sense sequence followed by a first antisense sequence complementary to the first sense sequence followed by a second cis-acting ribozyme followed by a third cis-acting ribozyme followed by a second sense sequence followed by a second antisense sequence complementary to the second sense sequence followed by a fourth cis-acting ribozyme.

The isolated nucleic acids provided herein can be designed such that a particular product is formed. For example, a single-stranded RNA containing a first cis-acting ribozyme followed by a sense sequence followed by a second cis-acting ribozyme followed by an antisense sequence followed by a third cis-acting ribozyme can result in multiple single strands of RNA. One strand can contain the sense sequence, and another strand can contain the antisense sequence. These two liberated strands can come together via their complementary sequence to form a double-stranded RNA molecule having the ability to induce RNA interference. It will be appreciated that a double-stranded RNA molecule contains two strands of RNA with each strand having its own 5′ and 3′ end.

In some embodiments, the sense and antisense sequences are liberated from a larger sequence as one piece of single-stranded RNA. The isolated nucleic acid can be designed such that the sense and antisense sequences of the resulting single-stranded RNA come together via their complementary sequence to form a hairpin loop structure having the ability to induce RNA interference. Any number of hairpin loop structures can be formed. For example, an isolated nucleic acid can be designed such that a resulting single-stranded RNA forms zero, one, two, three, four, five, six, seven, eight, nine, ten, or more hairpin loop structures. Typically, the resulting single-stranded RNA forms no more than one hairpin loop structure. In addition, the sense and antisense sequences that come together to form the stem portion of a hairpin loop structure are typically separated by 3 to 25 nucleotides (e.g., 3 to 20, 3 to 15, 3 to 10, 3 to 9, 5 to 15, or 5 to 10 nucleotides) that form the loop portion of a hairpin loop structure. For example, an isolated nucleic acid can be designed such that the sense and antisense sequences are separated by 5′-UUCAAGACC-3′ (SEQ ID NO: 8).

These resulting single- or double-stranded RNA molecules that are formed upon cleavage by one or more cis-acting ribozyme sequences can be enzymatically inactive (e.g., lack ribozyme activity such as trans-acting ribozyme activity). For example, a single-stranded RNA having no more than one hairpin loop structure can lack trans-acting ribozyme activity. Cis-acting ribozyme activity is measured using the following method. Briefly, in vitro transcription is performed using ³²P-labeled nucleotides, and the products are analyzed by polyacrylamide gel electrophoresis. The presence of cis-acting ribozyme activity is confirmed by observing the presence of defined cleavage products in a Mg-dependent manner. Trans-acting ribozyme activity is measured using the following method. Briefly, in vitro transcription is performed to generate RNA to be tested for trans-acting ribozyme activity. The generated RNA is then incubated with ³²P-labeled target RNA. The presence of trans-acting ribozyme activity is confirmed by observing the presence of cleaved target RNA in a Mg-dependent manner when analyzed by polyacrylamide gel electrophoresis.

The invention also provides mixtures having at least two isolated nucleic acids. In one embodiment, one of the isolated nucleic acids of the mixture can have one strand that contains (or is a template for) one or more sense sequences and one or more cis-acting ribozyme sequences, while a second isolated nucleic acid of the mixture has one strand that contains (or is a template for) one or more antisense sequences and one or more cis-acting ribozyme sequences. Other embodiments include, without limitation, mixtures of any two or more of the isolated nucleic acids provided herein. For example, a mixture can contain a first isolated nucleic acid having one strand that is a template for a sense sequence followed by an antisense sequence followed by a cis-acting ribozyme sequence in combination with a second isolated nucleic acid having one strand that is a template for a first cis-acting ribozyme followed by a sense sequence followed by a second cis-acting ribozyme sequence followed by an antisense sequence followed by a third cis-acting ribozyme sequence.

The isolated nucleic acids provided herein can contain a promoter sequence to promote transcription of an RNA molecule containing sense, antisense, and cis-acting ribozyme sequences. Any promoter sequence can be used. For example, a constitutive promoter (e.g., an SV40 promoter) or an inducible promoter (e.g., a tet-regulated promoter) can be used. In addition, the promoter sequence can be a promoter for RNA polymerase II (e.g., a CMV promoter sequence, an albumin promoter sequence, or a transferrin promoter sequence) or a promoter sequence for RNA polymerase III (e.g., a U6 promoter sequence, a U2 promoter sequence, or a val-tRNA promoter sequence). In some embodiments, a tissue-specific promoter, cell-specific promoter, or pathogen-specific promoter sequence can be used. Examples of such promoter sequences include, without limitation, a mouse albumin enhancer promoter, a transferrin promoter, a probasin promoter, a keratin 7 promoter, a keratin 13 promoter, a keratin enhancer promoter, and a whey acidic protein promoter. Other promoter sequences include those described in U.S. Pat. Nos. 5,824,519 and 6,271,359.

Tissue-specific, cell-specific, and pathogen-specific promoter sequences can be used to control the location of synthesis of RNA molecules having the ability to induce RNA interference. For example, an isolated nucleic acid having a liver-specific promoter (e.g., a mouse albumin enhancer promoter sequence) can be used to drive transcription of an RNA molecule containing sense, antisense, and cis-acting ribozyme sequences such that siRNA molecules are formed in liver cells. In one example, the antisense sequence can be a sequence complementary to an mRNA from liver or an mRNA from a hepatitis B virus (e.g., an mRNA encoding an envelope or polymerase/reverse transcriptase polypeptide) such that siRNA molecules targeting the liver or hepatitis B virus mRNA are formed. When targeting a sequence from an mRNA of a hepatitis B virus, sense or antisense sequences can be, without limitation, HBV₈₅₉₋₈₈₁, HBV₈₈₁₋₈₅₉, HBV₈₆₀₋₈₈₁, HBV₈₈₁₋₈₆₀, HBV₈₆₁₋₈₈₂, or HBV₈₈₂₋₈₆₁.

In another example, an isolated nucleic acid can be designed to generate, in skin cells (e.g., keratinocytes), RNA molecules that have the ability to induce RNA interference against a target mRNA from a human papilloma virus. In this case, an isolated nucleic acid having a skin-specific promoter (e.g., a keratin promoter sequence) can be used to drive transcription of an RNA molecule containing sense, antisense, and cis-acting ribozyme sequences such that RNA molecules having the ability to induce RNA interference are formed in skin cells. In addition, the antisense sequence can be a sequence complementary to an mRNA from a human papilloma virus (e.g., an mRNA encoding an E6 or E7 polypeptide such as the translational start site for an E6 or E7 polypeptide) such that RNA molecules having the ability to induce RNA interference against human papilloma virus mRNA are formed. When targeting a sequence from an mRNA of a human papilloma virus, sense and antisense sequences can be, without limitation, as set forth in SEQ ID NOs: 1-6.

The components of the isolated nucleic acids provided herein can be obtained using any method including, without limitation, common molecular cloning and chemical nucleic acid synthesis techniques. For example, PCR can be used to obtain a promoter sequence, a sense nucleic acid sequence, or a cis-acting ribozyme sequence. PCR refers to procedures in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complementary DNA strands.

In addition, mutagenesis (e.g., site-directed mutagenesis) can be used to obtain components of the isolated nucleic acids provided herein. For example, site-directed mutagenesis can be used to design particular sense and antisense sequences within a nucleic acid construct containing a template strand for one or more cis-acting ribozyme sequences. Possible mutations include, without limitation, deletions, insertions, and substitutions, as well as combinations of deletions, insertions, and substitutions. Nucleic acid and amino acid databases (e.g., GenBank®) also can be used to obtain sequence information such that particular sense and antisense sequences as well as promoter sequences and cis-acting ribozyme sequences can be obtained. For example, GenBank® can be used to design sense and antisense sequences such that siRNA molecules are formed that target a human mRNA sequence.

The isolate nucleic acids provided herein can be linear or circular. For example, an isolated nucleic acid can be in the form of a vector such as a plasmid, phage, or cosmid. As such, the isolated nucleic acids can contain an ori of replication, a sequence encoding a polypeptide that confers antibiotic resistance, and restriction enzyme sites. In some embodiments, an isolated nucleic acid provided herein is in the form of a vector derived from bacteriophage, baculoviruses, tobacco mosaic virus, herpes viruses, cytomegaloviruses, retroviruses, vaccinia viruses, adenoviruses, and/or adeno-associated viruses. Numerous vectors and expression systems are commercially available from, for example, Novagen (Madison, Wis.), Clontech (Palo Alto, Calif.), Stratagene (La Jolla, Calif.), and Invitrogen/Life Technologies (Carlsbad, Calif.).

Cells

The invention also provides cells containing one or more of the isolated nucleic acids provided herein. Such cells can be prokaryotic or eukaryotic. Examples of cells that can contain one or more of the isolated nucleic acids provided herein include, without limitation, animal cells, mammalian cells, plant cells, insect cells, fungal cells, bacterial cells, and yeast cells. Other example include, without limitation, primary cells, skin cells (e.g., keratinocytes), liver cells, neurons, muscle cells, lymphocytes, bone marrow cells, kidney cells, adrenal cells, T-cells, B-cells, macrophages, monocytes, adipocytes, perictyes, fibroblasts, colon epithelial cells, reticular cells, pancreatic cells, cervical cells, endometrial cells, and prostate cells. Cells containing at least one of the isolated nucleic acids provided herein can be in vitro or in vivo. In addition, the isolated nucleic acid can be integrated into the genome of the cell or maintained in an episomal state. Thus, cells can be stably or transiently transfected with a construct containing one or more of the isolated nucleic acids provided herein.

Any method can be used to introduce an isolated nucleic acid into a cell in vivo or in vitro. For example, calcium phosphate precipitation, electroporation, heat shock, lipofection, microinjection, and viral-mediated nucleic acid transfer are common methods that can be used to introduce an isolated nucleic acid into a cell. In addition, naked DNA can be delivered directly to cells in vivo as describe elsewhere (See, e.g., U.S. Pat. Nos. 5,580,859 and 5,589,466). Further, isolated nucleic acids can be introduced into cells by generating transgenic animals as described herein.

Any method can be used to identify cells containing an isolated nucleic acid provided herein. Such methods include, without limitation, PCR and nucleic acid hybridization techniques such as Northern and Southern analysis.

Viruses

The invention provides viruses containing one or more of the isolated nucleic acids provided herein. Examples of viruses that can contain one or more of the isolated nucleic acids provided herein include, without limitation, retroviruses, adenoviruses, herpes viruses, adeno-associated viruses, lentiviruses, baculoviruses, cauliflower mosaic viruses, tobacco mosaic viruses, togaviruses, polioviruses, cytomegaloviruses, Paramyxoviruses, Epstein-Barr viruses, human papillomavirus, and hepatitis C viruses.

A virus containing one or more of the isolated nucleic acids provided herein can be used as a viral vector to package and/or deliver the isolated nucleic acids to a tissue, cell, pathogen, bacteria, virus, or fungus. For example, a retroviral vector can be used to deliver an isolated nucleic acid provided herein to cells in vivo or ex vivo. Other examples of viral vector that can be used to deliver one or more of the isolated nucleic acids provided herein include, without limitation, those described in WO 97/17458 and U.S. Pat. No. 6,271,359.

Any method can be used to introduce an isolated nucleic acid into a virus. For example, common molecular cloning techniques can be used to introduce an isolated nucleic acid provided herein into the sequence of a virus. In addition, standard virology techniques can be used to generate and isolate virus particles containing one or more of the isolated nucleic acids provided herein.

Any method can be used to identify viruses containing an isolated nucleic acid provided herein. Such methods include, without limitation, PCR and nucleic acid hybridization techniques such as Northern and Southern analysis. For example, cells infected with a particular virus can be analyzed for the presence or absence of nucleic acid corresponding to an isolated nucleic acid provided herein.

Transgenic Animals

The invention provides non-human transgenic animals containing one or more of the isolated nucleic acids provided herein. Such transgenic animals can be aquatic animals (such as fish, sharks, dolphin, and the like), farm animals (such as pigs, goats, sheep, cows, horses, rabbits, and the like), rodents (such as rats, guinea pigs, and mice), non-human primates (such as baboon, monkeys, and chimpanzees), and domestic animals (such as dogs and cats). Several techniques known in the art can be used to introduce an isolated nucleic acid into animals to produce the founder lines of transgenic animals. Such techniques include, without limitation, pronuclear microinjection (U.S. Pat. No. 4,873,191); retrovirus mediated gene transfer into germ lines (Van der Putten et al., Proc. Natl. Acad. Sci., USA, 82:6148 (1985)); gene transfection into embryonic stem cells (Gossler A et al., Proc Natl Acad Sci USA 83:9065-9069 (1986)); gene targeting into embryonic stem cells (Thompson et al., Cell, 56:313 (1989)); nuclear transfer of somatic nuclei (Schnieke A E et al., Science 278:2130-2133 (1997)); and electroporation of embryos (Lo C W, Mol. Cell. Biol., 3:1803-1814 (1983)). Once obtained, transgenic animals can be replicated using traditional breeding or animal cloning.

The transgenic animals provided herein can be designed to be functional knock-outs of the polypeptide encoded by the mRNA that the siRNA molecules target for degradation. Such transgenic animals can be used to screen compounds for their ability restore the function or functions of the polypeptide knocked out. In general, such screening methods can include administering a test compound to the transgenic animal and determining if the function of the knocked out polypeptide is fully or partially restored relative to a control transgenic animal that did not receive the test compound.

Any method can be used to identify transgenic animals containing an isolated nucleic acid provided herein. Such methods include, without limitation, PCR and nucleic acid hybridization techniques such as Northern and Southern analysis.

Methods

The invention provides methods for reducing the level of RNA (e.g., an mRNA) within a cell. Such methods typically involve introducing one or more of the isolated nucleic acids provided herein into a cell. The invention also provides methods for inducing RNA interference in a cell by introducing an isolated nucleic acid into the cell such that a single-stranded RNA having no more than one hairpin loop structure is formed within the cell as a result of cleavage by at least one cis-acting ribozyme.

In general, the isolated nucleic acid introduced into a cell is designed such that the sense and antisense sequences target an mRNA normally within that cell. In some embodiments, the isolated nucleic acid introduced into a cell can be designed such that the sense and antisense sequences target an mRNA not normally within that cell. For example, the sense and antisense sequences can target an RNA sequence from a virus. In this case, the isolated nucleic acid can be used to protect cells from infection and/or lysis normally caused by that virus.

In some embodiment, the methods and materials provided herein can be used to treat conditions arising from (1) expression of an abnormal polypeptide (e.g., a mutated or dysfunctional polypeptide) or (2) abnormal expression (e.g., over-expression) of a normal polypeptide. For example, an isolated nucleic acid provided herein can be designed such that the sense and antisense sequences target an mRNA encoding a particular polypeptide. Once designed, the isolated nucleic acid can be administered to a subject (e.g., a human) suspected of having a disease or condition that can be alleviated by down-regulating the expression of the polypeptide encoded by the targeted mRNA. Examples of diseases and conditions that can be treated using an isolated nucleic acid provided herein include, without limitation, bacterial infections (e.g., E. coli, S. aureus, and P. aeruginosa infections), viral infections (e.g., papilloma virus, hepatitis virus, herpes virus, and HIV infections), proliferative disease (e.g., cancers such as lymphomas, breast cancers, prostrate cancers, cervical cancers, melanomas, neuroblastomas, testicular cancers, and ovarian cancers), and inflammatory diseases (e.g., arthritis and asthma).

To treat bacterial and viral infections, the isolated nucleic acids can be designed such that the sense and antisense sequences target mRNA that encodes an indispensable polypeptide from the bacterium or virus. For example, an isolated nucleic acid can be designed such that the sense and antisense sequences target mRNA that encodes a bacterial enzyme required for the bacterium's survival. When treating bacterial infections, the isolated nucleic acids can be delivered directly to the bacteria using, for example, bacteriophage vectors. When treating viral infections, the isolated nucleic acids can be delivered to the infected cells or the cells susceptible to infection using, for example, viral vectors having a tropism for those cells.

To treat proliferative diseases such as cancers, the isolated nucleic acids can be designed such that the sense and antisense sequences target mRNA that encodes an oncogene product (e.g., ras, myc, Src, myb, and Wnt). In addition, the isolated nucleic acids can be delivered to the cancerous cells via intratumoral injection or through the use of viral vectors having a tropism for the cancerous cells.

To treat inflammatory conditions, the isolated nucleic acids can be designed such that the sense and antisense sequences target mRNA that encodes a pro-inflammatory cytokine (e.g., interferon-γ, TNF-α, TGF-β, and interleukins such as IL-11). In addition, the isolated nucleic acids can be delivered to the cells at the site of inflammation via localized injections or through the use of viral vectors having a tropism for the pro-inflammatory cells (e.g., macrophages and lymphocytes) at the site of inflammation.

Screening Methods and Materials

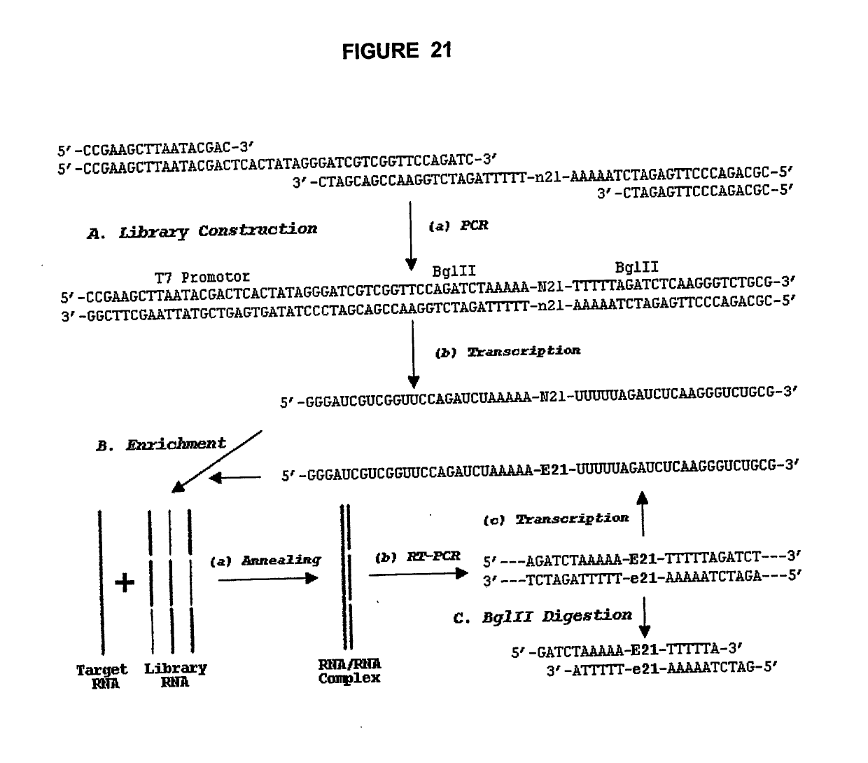

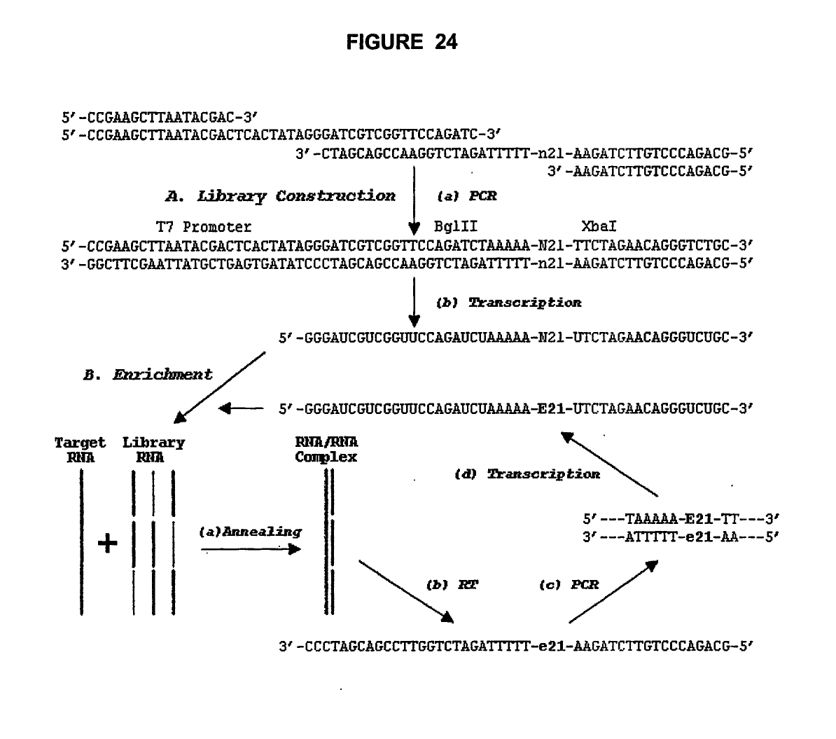

The invention provides in vitro and in vivo screening methods and materials that can be used to identify mRNA sequences that can be targeted for RNA interference. For example, the following in vitro screen can be performed as outlined in FIG. 21 or 24 to identify regions of an mRNA that are available for hybridization. Briefly, PCR can be used to construct a pool of nucleic acids with each member containing a sequence of between about 10 and about 30 (e.g., 11, 13, 15, 17, 19, 21, 23, 25, 27, or 29) randomized nucleotides located downstream from a promoter sequence (e.g., a T7 promoter) and between two restriction enzyme sites (e.g., two BglII sites). After transcription, the transcripts containing the randomized sequences can be annealed to a target mRNA and RT-PCR is performed. After amplification, the amplification products can be cleaved with the restriction enzymes (e.g., BglII), and the cleavage products can be cloned into vectors linearized to receive the cleaved products. The resulting collection of vectors can contain sequences that are a template for RNA molecules having the ability to bind to the mRNA sequence used as the selection target. These inserts can be sequenced and used to design the sense and antisense sequences provided herein such that RNA interference in induced. Each step of this selection protocol can be performed used methods similar to those described in U.S. Provisional Patent Application Ser. No. 60/417,997, PCT Application Publication WO 04/002416, Pan et al. (Molecular Therapy, 7:129-139 (2003)), and Pan et al. (RNA, 7:610-621 (2001)). These in vitro screening methods can be modified to add multiple rounds of enrichment (e.g., two, three, four, five, six, or more rounds of enrichment). A binding test can be performed with labeled selected library transcripts and labeled unselected library transcripts to verify that the transcripts from the enriched library have increased affinity for the target mRNA.

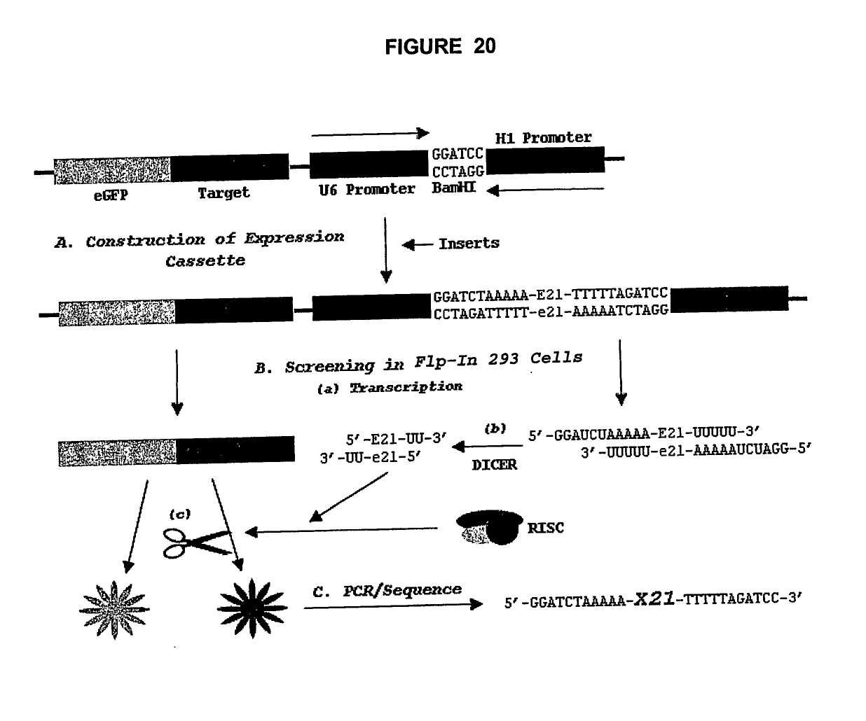

Collections of sequences such as the collection of sequences obtained using an in vitro screen can be subjected to an in vivo screen to obtain large numbers of sequences that can be used to induce RNA interference in an efficient manner (FIG. 20). In addition, individual sequences can be subjected to an in vivo screen to confirm that the individual sequence has the ability to induce RNA interference (FIG. 20). Briefly, an in vivo screen can involve introducing a vector preparation into cells. Each vector of the vector preparation can contain two main components: (1) a nucleic acid sequence that is a template for a target mRNA and (2) a nucleic acid sequence that is a template for a double-stranded RNA molecule to be tested for the ability to induce RNA interference such that the levels of the target mRNA and encoded polypeptide are reduced. In some embodiments, the cells can contain the target mRNA, and each vector of the vector preparation can contain one main component: a nucleic acid sequence that is a template for a double-stranded RNA molecule to be tested for the ability to induce RNA interference such that the levels of the target mRNA and encoded polypeptide are reduced. Such cells can contain the target mRNA endogenously. In some cases, the cells can be provided with a nucleic acid sequence that encodes the target mRNA. For example, cells can be transfected with an expression vector that encodes a target mRNA.

To aid in the assessment of the level of target mRNA and encoded polypeptide, the nucleic acid sequence that is a template for the target mRNA can be fused to a sequence encoding a reporter polypeptide (e.g., green fluorescent protein or red fluorescent protein) such that the target mRNA and the mRNA encoding the reporter polypeptide are transcribed as a single fusion mRNA. Any polypeptide can be used as a reporter polypeptide. In some embodiments, the reporter polypeptide can be a polypeptide that kills cells when expressed. For example, the reporter polypeptide can be lethal to cells or can be a polypeptide that induces lethal responses (e.g., a polypeptide that activates a lethal polypeptide such as caspase 3). In other embodiments, target mRNA can be transcribed without a sequence encoding a reporter polypeptide. It is noted that transcription of the fusion mRNA (or target mRNA) can be driven by any type of promoter sequence. For example, each vector of the vector preparation can contain a CMV promoter sequence operably linked to the sequence that is a template for the fusion mRNA.

When using a reporter polypeptide (e.g., green fluorescent protein or red fluorescent protein) to identify cells with reduced levels of target mRNA, a promoter sequence that promotes transcription to a lesser degree than a strong promoter such as a CMV promoter can be used. Using a weak promoter sequence can allow researchers to distinguish easily cells exhibiting effective target mRNA reduction from those cells that do not reduce target mRNA levels. The U6 promoter sequence is an example of a weak promoter that can allow researchers to distinguish easily cells exhibiting effective target mRNA reduction.

The portion of each vector that contains the nucleic acid sequence that is a template for a double-stranded RNA molecule to be tested can be arranged such that a first promoter sequence (e.g., a U6, H1, or CMV promoter sequence) directs transcription from the top strand and a second promoter sequence (e.g., a U6, H1, or CMV promoter sequence) directs transcription from the bottom strand (FIG. 20). In this case, the two resulting transcripts can anneal to from a double-stranded RNA molecule. Each nucleic acid sequence that is a template for a double-stranded RNA molecule to be tested can be positioned between the two promoter sequences using restriction enzyme sites. The nucleic acid sequence between the two promoter sequences can be any length. For example, the nucleic acid sequence between the two promoter sequences can be from about 15 to about 300 nucleotides in length. In some embodiments, the nucleic acid sequence between the two promoter sequences is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some cases, the two promoter sequences can be different. For example, one promoter sequence can be a U6 promoter sequence, while the other can be an H1 promoter sequence. The use of two different promoter sequences can simplify the sequencing procedures used to determine the sequence located between the two promoter sequences. For example, a sequencing primer designed to anneal to one of the promoter sequences will not anneal to the other promoter sequence when two different promoter sequences are used.

The two promoter sequences can direct transcription of two transcripts: one from the top strand, and one from the bottom strand. If the two transcripts form double-stranded RNA molecules that induce degradation of the target mRNA, then the level of the reporter polypeptide expressed by the cell will be lower than the level observed in control cells (e.g., cells lacking the ability to transcribe both transcripts). The cells exhibiting a reduced level of reporter polypeptide can be isolated. Once a cell is isolated, the vector sequences responsible for the observed reduced level can be identified and used to design sense and antisense sequences as described herein.

In one embodiment, a nucleic acid sequence that is a template for a target mRNA is fused to a sequence that encodes eGFP. See, e.g., Jang et al., J. Virol. 62:2636-2643 (1988). The resulting construct can be cloned into a vector (e.g., the pcDNA5 vector; Invitrogen, Carlsbad, Calif.) such that transcription is under the control of a CMV or U6 promoter. A construct having two U6 promoter sequences (or one U6 promoter sequence and one H1 promoter sequence) with one promoter sequence driving transcription from the top strand and the other driving transcription from the bottom strand can be made such that one or more restriction enzyme sites are located between the two promoter sequences. This construct can be used to clone in sequences to be tested. For example, nucleic acids obtained using an in vitro screen can be cloned into a restriction site (e.g., a BamHI site) to produce a collection of constructs containing each member of the nucleic acids obtained using an in vitro screen. This resulting collection of constructs can be cloned into the pcDNA5 vector containing the nucleic acid sequence that is a template for the target mRNA fused to the sequence that encodes eGFP to produce a library.

The library can be used to transfect human 293 FlipIn™ cells (Invitrogen, Carlsbad, Calif.) such that a construct containing the component(s) described herein integrates into the recombination site of the human 293 FlipIn™ cells as a single copy. Transformants can be selected using Hygromycin antibiotic and screened for a reduction or elimination of fluorescence. Cells that do not fluoresce can contain a single copy of the integrated construct. The sequence of this integrated construct can be sequenced and used to design sense and antisense sequences as described herein.

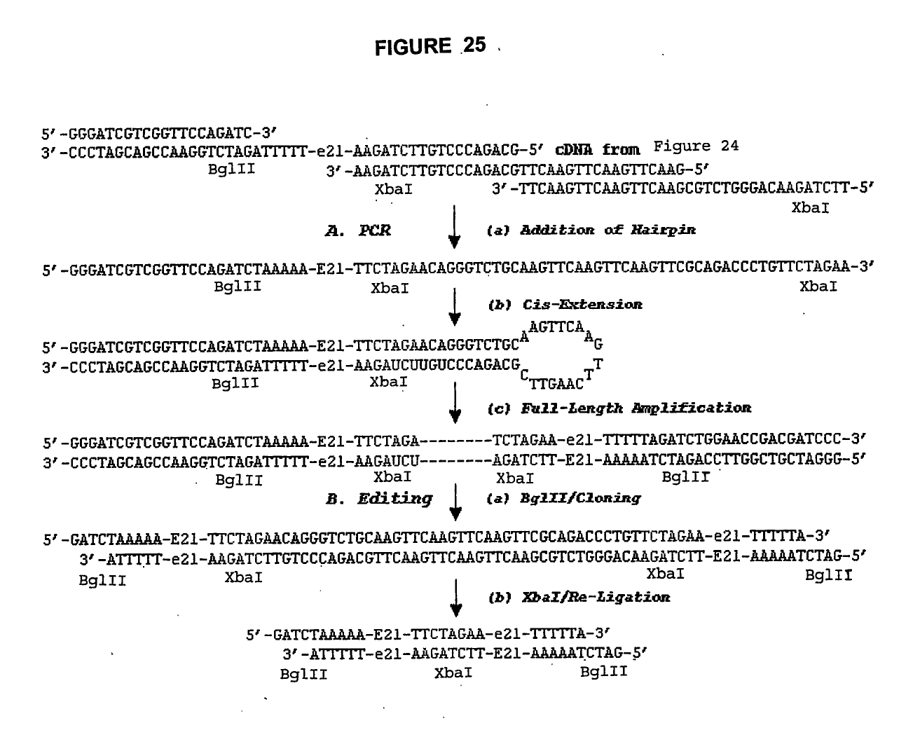

Collections of sequences such as the collection of sequences obtained using an in vitro screen (FIG. 24) can be subjected to a cloning process that produces a library of sequences that encode hairpin loop sequences (FIG. 25). Such hairpin loop sequences can be designed such that (1) the stem portion of the hairpin loop is formed, at least in part, by sense and antisense sequences, and (2) the loop portion of the hairpin loop is formed by a sequence located between the sense and antisense sequences. The loop portion can be any size (e.g., 3, 4, 5, 6, 7, 8, 9, 10, or more nucleotides) and can have any sequence. The hairpin loop sequences can be used to induce RNA interference.

In one embodiment, the following cloning procedure can be used to produce a library of sequences that encode hairpin loop sequences (FIG. 25). Briefly, a library of cDNA sequences from, for example, an in vitro screen such as the one depicted in FIG. 24 can be combined with three PCR primers. Such cDNA sequences can contain a restriction site (e.g., BglII) 3′ of the random sequences (e.g., e21) and a different restriction site (e.g., XbaI) 5′ of the random sequences (e.g., e21). The first PCR primer (first strand of FIG. 25) can be complementary to the 3′ end of the cDNA sequences. The second PCR primer (third strand of FIG. 25) can contain a sequence that overlaps with the 5′ end of the cDNA sequences and a sequence that extends beyond the 5′ end of the cDNA sequences. The sequence that extends beyond the 5′ end of the cDNA sequences can be designed to form a loop structure. The third PCR primer (fourth strand of FIG. 25) can contain a sequence that overlaps with the 5′ end of the second PCR primer and a sequence that extends beyond the 5′ end of the second PCR primer. The sequence that extends beyond the 5′ end of the second PCR primer can contain the same restriction site (e.g., XbaI) that is located in the cDNA sequences 5′ of the random sequences (e.g., e21).

After amplification (step A(a) of FIG. 25), the sequences from the cDNA sequences and PCR primers can be incorporated into a larger product. The 3′ end of this product can fold onto itself and allow for cis-extension during a PCR reaction. This reaction can be performed using the first PCR primer. This primer can be used at a concentration that is higher than normal. For example, 700 pM of the first primer can be used in this cis-extension PCR reaction as opposed to the 100 pM amount normally used in PCR reactions.

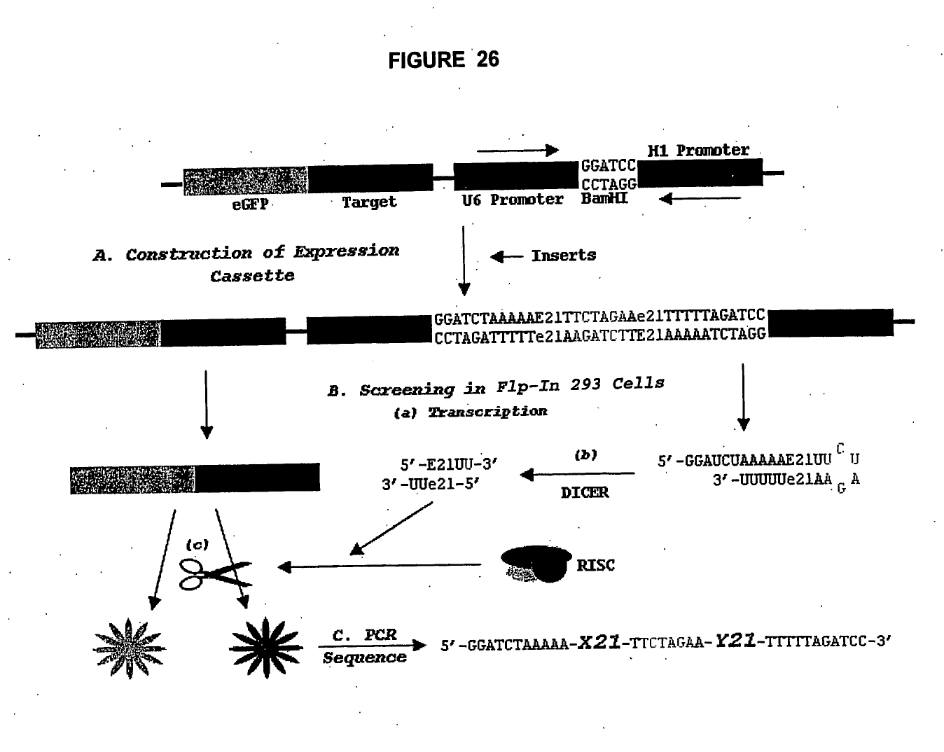

After amplifying the extended products (step A(c) of FIG. 25), the products can be edited to remove at least a portion of the sequence (e.g., most of the sequence) located between the sense and antisense sequences (e.g., E21 and e21) present in each strand. Briefly, the amplified products can be cleaved at the flanking outer restriction sites (e.g., BglII sites) and cloned into a vector (e.g., a pCR2 vector). The vector can be modified or designed to lack restriction sites that are cleaved by the restriction enzyme that cleaves at the inner flanking restriction sites (e.g., XbaI). After amplifying the vectors, the vectors can be cleaved at the inner flanking restriction sites (e.g., XbaI). The excised fragment can be discarded, and the vector fragments can be re-ligated. The sequences located between the outer flanking restriction sites can be excised and cloned into any type of vector such as an in vivo screening vector (FIG. 26).

The library of sequences that encode hairpin loop sequences can be subjected to an in vivo screen to obtain large numbers of sequences that can be used to induce RNA interference in an efficient manner (FIG. 26). In addition, individual sequences encoding a hairpin loop sequence can be subjected to an in vivo screen to confirm that the individual sequence has the ability to induce RNA interference (FIG. 26). Briefly, an in vivo screen can involve introducing a vector preparation into cells. Each vector of the vector preparation can contain two main components: (1) a nucleic acid sequence that is a template for a target mRNA and (2) a nucleic acid sequence that is a template for an RNA molecule with a hairpin loop sequence to be tested for the ability to induce RNA interference such that the levels of the target mRNA and encoded polypeptide are reduced. In some embodiments, the cells can contain the target mRNA, and each vector of the vector preparation can contain one main component: a nucleic acid sequence that is a template for an RNA molecule with a hairpin loop sequence to be tested for the ability to induce RNA interference such that the levels of the target mRNA and encoded polypeptide are reduced. Such cells can contain the target mRNA endogenously. In some cases, the cells can be provided with a nucleic acid sequence that encodes the target mRNA. For example, cells can be transfected with an expression vector that encodes a target mRNA.

To aid in the assessment of the level of target mRNA and encoded polypeptide, the nucleic acid sequence that is a template for the target mRNA can be fused to a sequence encoding a reporter polypeptide (e.g., green fluorescent protein or red fluorescent protein) such that the target mRNA and the mRNA encoding the reporter polypeptide are transcribed as a single fusion mRNA. Any polypeptide can be used as a reporter polypeptide. In some embodiments, the reporter polypeptide can be a polypeptide that kills cells when expressed. For example, the reporter polypeptide can be lethal to cells or can be a polypeptide that induces lethal responses (e.g., a polypeptide that activates a lethal polypeptide such as caspase 3). In other embodiments, target mRNA can be transcribed without a sequence encoding a reporter polypeptide. It is noted that transcription of the fusion mRNA (or target mRNA) can be driven by any type of promoter sequence. For example, each vector of the vector preparation can contain a CMV promoter sequence operably linked to the sequence that is a template for the fusion mRNA.

When using a reporter polypeptide (e.g., green fluorescent protein or red fluorescent protein) to identify cells with reduced levels of target mRNA, a promoter sequence that promotes transcription to a lesser degree than a strong promoter such as a CMV promoter can be used. Using a weak promoter sequence can allow researchers to distinguish easily cells exhibiting effective target mRNA reduction from those cells that do not reduce target mRNA levels. The U6 promoter sequence is an example of a weak promoter that can allow researchers to distinguish easily cells exhibiting effective target mRNA reduction.

The portion of each vector that contains the nucleic acid sequence that is a template for an RNA molecule with a hairpin loop sequence to be tested can be arranged such that a single promoter sequence (e.g., a U6, H1, or CMV promoter sequence) directs transcription from either the top or bottom strand. In some embodiments, the portion of each vector that contains the nucleic acid sequence that is a template for an RNA molecule with a hairpin loop sequence to be tested can be arranged such that a first promoter sequence (e.g., a U6, H1, or CMV promoter sequence) directs transcription from the top strand and a second promoter sequence (e.g., a U6, H1, or CMV promoter sequence) directs transcription from the bottom strand (FIG. 26). In such cases, the two resulting transcripts can form separate hairpin loop structures. Each nucleic acid sequence that is a template for an RNA molecule with a hairpin loop sequence to be tested can be positioned behind a promoter sequence or between two promoter sequences using restriction enzyme sites. The nucleic acid sequence behind a promoter sequence or between two promoter sequences can be any length. For example, the nucleic acid sequence between two promoter sequences can be from about 15 to about 300 nucleotides in length.

In some cases, the two promoter sequences can be different. For example, one promoter sequence can be a U6 promoter sequence, while the other can be an H1 promoter sequence. The use of two different promoter sequences can simplify the sequencing procedures used to determine the sequence located between the two promoter sequences. For example, a sequencing primer designed to anneal to one of the promoter sequences will not anneal to the other promoter sequence when two different promoter sequences are used.

Once transcribed from the top and/or bottom strand, the RNA molecules can form a hairpin loop structure. If a transcript forms a structure that induces degradation of the target mRNA, then the level of the reporter polypeptide expressed by the cell will be lower than the level observed in control cells (e.g., cells lacking the ability to transcribe the RNA molecules). The cells exhibiting a reduced level of reporter polypeptide can be isolated. Once a cell is isolated, the vector sequences responsible for the observed reduced level can be identified and used to design sense and antisense sequences as described herein.

Nucleic Acid Delivery

As described herein, any method can be used to deliver an isolated nucleic acid to a cell. In some embodiments, delivery of an isolated nucleic acid provided herein can be performed via biologic or abiologic means as described in, for example, U.S. Pat. No. 6,271,359. Abiologic delivery can be accomplished by a variety of methods including, without limitation, (1) loading liposomes with an isolated nucleic acid provided herein and (2) complexing an isolated nucleic acid with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged nucleic acids to form liposomes. Examples of cationic liposomes include lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

In some embodiments, systemic delivery is optimized using commercially available cationic lipids such as DDAB or DOTAP, each of which can be mixed with a neutral lipid such as DOPE or cholesterol. In some cases, liposomes such as those described by Templeton et al. (Nature Biotechnology, 15:647-652 (1997)) can be used. In other embodiments, polycations such as polyethyleneimine can be used to achieve delivery in vivo and ex vivo (Boletta et al., J. Am Soc. Nephrol. 7: 1728 (1996)). Additional information regarding the use of liposomes to deliver isolated nucleic acids can be found in U.S. Pat. No. 6,271,359.

The mode of delivery can vary with the targeted cell or tissue. For example, isolated nucleic acids can be delivered to lung and liver tissue to treat a disease (e.g., cancer, tuberculosis, and hepatitis) via the intravenous injection of liposomes since both lung and liver tissue take up liposomes in vivo. In addition, when treating localized conditions such as cancer and infections (e.g., hepatitis), catheritization in an artery upstream of the affected organ can be used to deliver liposomes containing an isolated nucleic acid. This catheritization can avoid clearance of the liposomes from the blood by the lungs and/or liver. For lesions such as skin cancer, human papilloma virus lesions, herpes lesions, and precancerous cervical dysplasia, topical delivery of liposomes can be used. Leukemias can be treated by ex vivo administration of the liposomes to, for example, to bone marrow.

Liposomes containing an isolated nucleic acid provided herein can be administered parenterally, intravenously, intramuscularly, intraperitoneally, transdermally, excorporeally, or topically. The dosage can vary depending on the species, age, weight, condition of the subject, and the particular compound delivered.

In other embodiments, biologic delivery vehicles can be used. For example, viral vectors can be used to deliver an isolated nucleic acid to a desired target cell. Standard molecular biology techniques can be used to introduce one or more of the isolated nucleic acids provided herein into one of the many different viral vectors previously developed to deliver nucleic acid to particular cells. These resulting viral vectors can be used to deliver the one or more isolated nucleic acids to the targeted cells by, for example, infection.

Pharmaceutical Compositions

Typically, one or more of the isolated nucleic acids provided herein, either alone or in combination with a biologic or abiologic delivery vehicle, can be administered to a subject suspected of having a disease or condition associated with the expression of a target polypeptide. The isolated nucleic acids and/or delivery vehicles can be in a pharmaceutically acceptable carrier or diluent, and can be administered in amounts and for periods of time that will vary depending upon the nature of the particular disease, its severity, and the subject's overall condition. Typically, an isolated nucleic acid is administered in an inhibitory amount such as an amount that is effective for inhibiting the production of the target polypeptide.

The ability of an isolated nucleic acid to induce RNA interference against a target can be assessed, for example, by measuring mRNA or polypeptide levels in a subject before and after treatment. Any method can be used to measure mRNA and polypeptide levels in tissues or biological samples such as Northern blots, RT-PCR, immunostaining, ELISAs, and radioimmunoassays.

Any method can be used to formulate and subsequently administer a composition containing one or more of the isolated nucleic acids provided herein. Dosing is generally dependent on the severity and responsiveness of the disease state to be treated, with the course of treatment lasting from several days to several months, or until a cure is effected or a diminution of the disease state is achieved. Routine methods can be used to determine optimum dosages, dosing methodologies, and repetition rates. Optimum dosages can vary depending on the relative potency of individual nucleic acids, and can generally be estimated based on EC₅₀ values found to be effective in in vitro and/or in vivo animal models. Typically, dosage is from about 0.01 μg to about 100 g per kg of body weight, and can be given once or more daily, weekly, or even less often. Following successful treatment, it may be desirable to have the patient undergo maintenance therapy to prevent the recurrence of the disease state.

Compositions containing one or more of the isolated nucleic acids provided herein can be admixed, encapsulated, conjugated, or otherwise associated with other molecules, molecular structures, or mixtures of nucleic acids such as, for example, liposomes, receptor targeted molecules, or oral, rectal, topical or other formulations, for assisting in uptake, distribution, and/or absorption.

Compositions containing one or more of the isolated nucleic acids provided herein can contain one or more pharmaceutically acceptable carriers. A “pharmaceutically acceptable carrier” (also referred to herein as an “excipient”) is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The compositions provided herein can be administered by a number of methods depending upon whether local or systemic treatment is desired and upon the area to be treated. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, the composition can be administered by injection or infusion into the cerebrospinal fluid, preferably with one or more agents capable of promoting penetration across the blood-brain barrier.

Compositions for topical administration include, for example, sterile and non-sterile aqueous solutions, non-aqueous solutions in common solvents such as alcohols, or solutions in liquid or solid oil bases. Such solutions also can contain buffers, diluents, and other suitable additives. Compositions for topical administration can formulated in the form of transdermal patches, ointments, lotions, creams, gels, drops, suppositories, sprays, liquids, and powders. Coated condoms, gloves, and the like also can be used. Conventional pharmaceutical carriers, aqueous, powder or oily bases, thickeners, and the like can be added.

Compositions for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Compositions for parenteral, intrathecal, or intraventricular administration can include, for example, sterile aqueous solutions, which also can contain buffers, diluents, and other suitable additives (e.g., penetration enhancers, carrier compounds, and other pharmaceutically acceptable carriers).

In some embodiments, a composition containing one or more of the isolated nucleic acids provided herein can contain other therapeutic agents such as anti-inflammatory drugs (e.g., nonsteroidal anti-inflammatory drugs and corticosteroids) and antiviral drugs (e.g., ribivirin, vidarabine, acyclovir, and ganciclovir).

The invention will be further described in the following examples, which do not limit the scope of the invention described in the claims.

### EXAMPLES

### Example 1

### Construction of pCLIP Cassettes Containing Human Papilloma Virus (HPV) Sequences

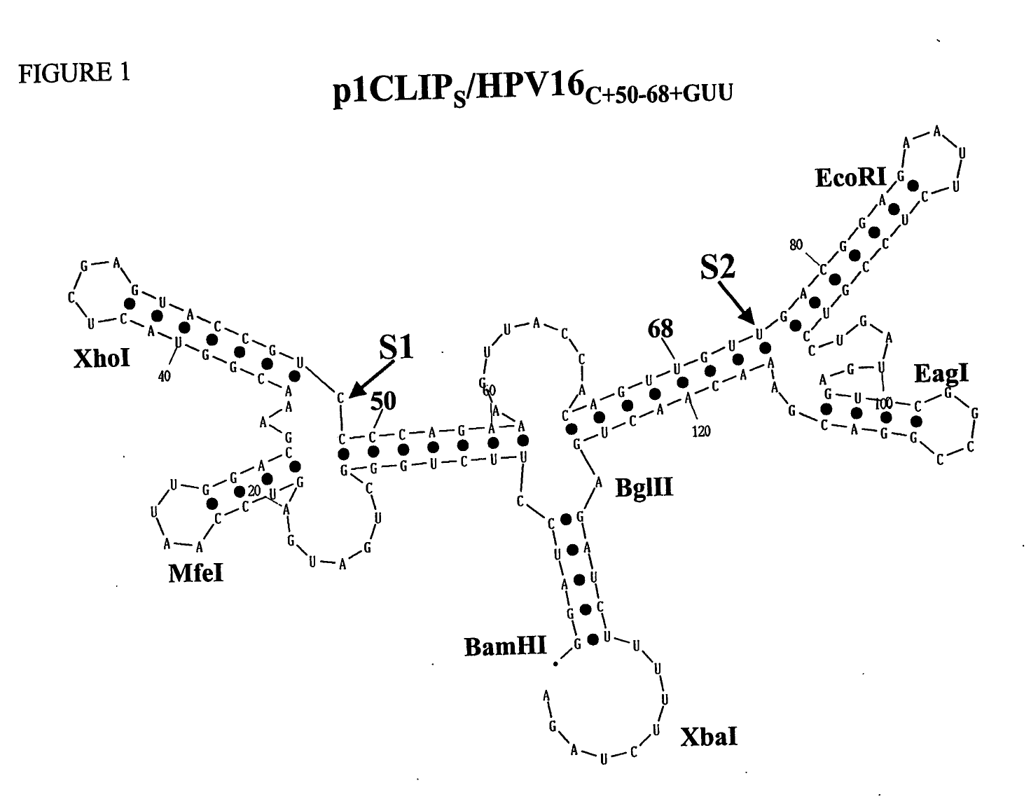

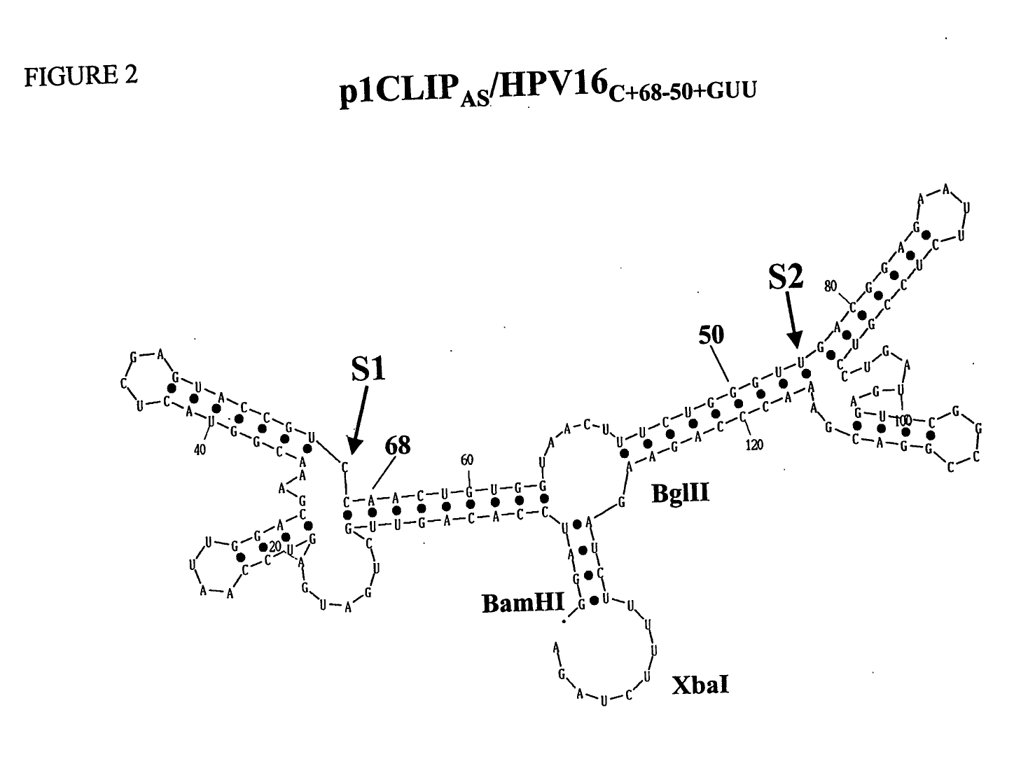

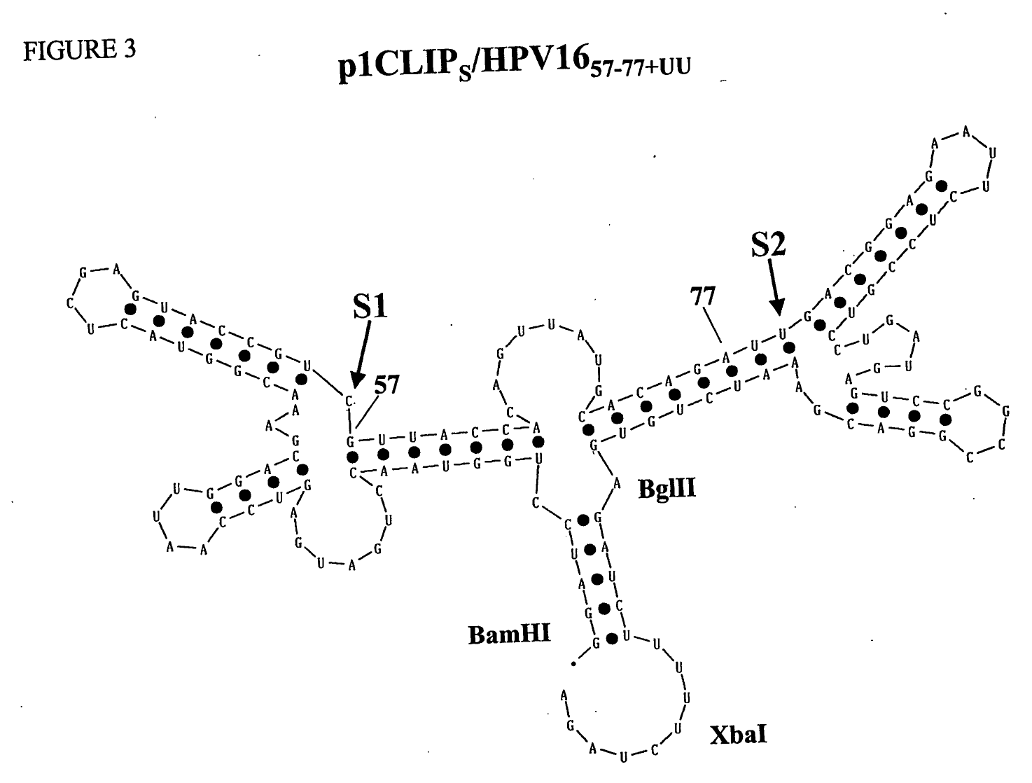

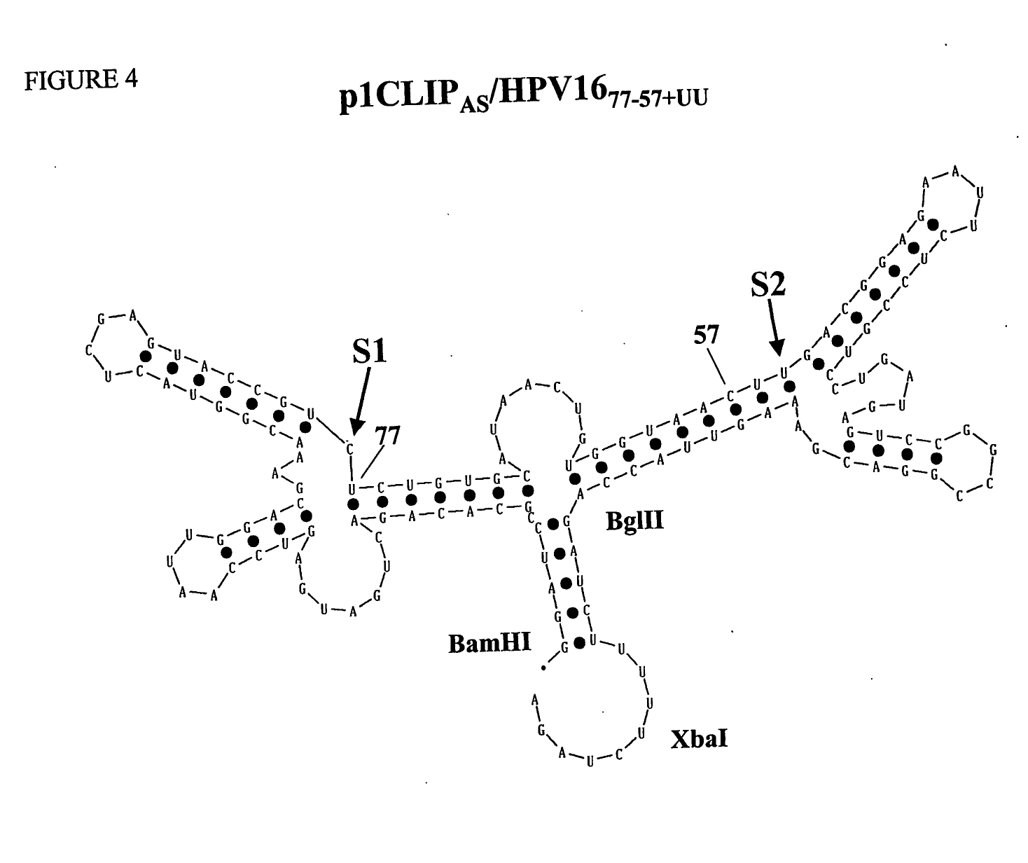

Four p1CLIP cassettes were constructed to contain one strand that is a template for (1) a cis-acting ribozyme sequence followed by (2) either a sense or antisense sequence followed by (3) a cis acting ribozyme sequence (p1CLIP_{S}/HPV16_{C+50-68(H)+GUU}; p1CLIP_{AS}/HPV16_{C+68-50(H)+GUU}; p1CLIP_{S}/HPV16_{57-77+UU}; and p1CLIP_{AS}/HPV16_{77-57+UU}; FIGS. 1-4). The HPV16_{50-68(H)} sequence is 5′-CCGGAAAGTTACCACAGTT-3′ (SEQ ID NO: 9), while the HPV16₅₇₋₇₇ sequence is 5′-GTTACCACAGTTATGCACAGA-3′ (SEQ ID NO: 10). The HPV16 sequences used herein were obtained from the Hershey strain of HPV16. See, GenBank® Accession Nos. gi3377787, 2196720, 1098775, and 1098731. In some cases, the HPV16 sequences correspond to the HPV16 sequences found in GenBank® Accession No. gi333031. In those cases, the HPV16₅₀₋₆₈ sequence would be 5′-CCAGAAA-GTTACCACAGTT-3′ (SEQ ID NO: 11).

FIGS. 1-4 contain the predicted structure (mFold program; see, e.g., Zuker and Jacobson, RNA, 4:669-679 (1998)) of the RNA molecules transcribed from p1CLIP_{S}/HPV16_{C+50-68+GUU}; p1CLIP_{AS}/HPV16_{C+68-50+GUU}; p1CLIP_{S}/HPV16_{57-77+GUU}; and p1CLIP_{AS}/HPV16_{77-57+GUU} respectively. A portion of p1CLIP_{S}/HPV16_{C+50-68(H)+GUU} contains the following sequence: 5′-GGATCCTTCCGGGCTGATGAGTCCAATTGGACGAAACGGTACTCGAGTACCGTCCCCGGAAAGTTACCACAGTTGTTGACGGAGAATTCTCCGTCCTGATGAGTCCGGCCGGACGAAACAACTGAGATCTTTTTCTAGA-3′ (SEQ ID NO: 12). A portion of p1CLIP_{AS}/HPV16_{C+68-50(H)+GUU} contains the following sequence: 5′-GGATCCACAGTTGCTGATGAGTCCAATTGGACGAAACGGTACTCGAGTACCGTCCAACTGTGGTAACTTTCCGGGTTGACGGAGAATTCTCCGTCCTGATGAGTCCGGCCGGACGAAACCCGGAAGATCTTTTTCTAGA-3′ (SEQ ID NO: 13). A portion of p1CLIP_{S}/HPV16_{57-77+UU} contains the following sequence: 5′-GGATCCTGGTAACCTGATGAGTCCAATTGGACGAAACGGTACTCGAGTACCGTCGTTACCACAGTTATGCACAGATTGACGGAGAATTCTCCGTCCTGATGAGTCCGGCCGGACGAAATCTGTGAGATCTTTTTCTAGA-3′ (SEQ ID NO: 14). A portion of p1CLIP_{AS}/HPV16_{77-57+UU} contains the following sequence: 5′-GGATCCGCACAGACTGATGAGTCCAATTGGACGAAACGGTACTCGAGTACCGTCTCTGTGCATAACTGTGGTAACTTGACGGAGAATTCTCCGTCCTGATGAGTCCGGCCGGACGAAAGTTACCAGATCTTTTTCTAGA-3′ (SEQ ID NO: 15).

In each case, the middle underlined sequence is either the sense or antisense sequence that corresponds to an HPV sequence. The first underlined sequence is complementary to a first region of the sense or antisense sequence and helps position the cis-acting ribozyme cleavage site within a few nucleotides of the sense or antisense sequence. The last underlined sequence is complementary to a last region of the sense or antisense sequence and helps position the cis-acting ribozyme cleavage site within a few nucleotides of the sense or antisense sequence. In some cases, the cassette can be designed such that the sense and antisense sequences are further away from the cleavage sites. For example, the sense and antisense sequences can be designed to be entirely within the bulge shown in FIG. 1 at positions 62-69.

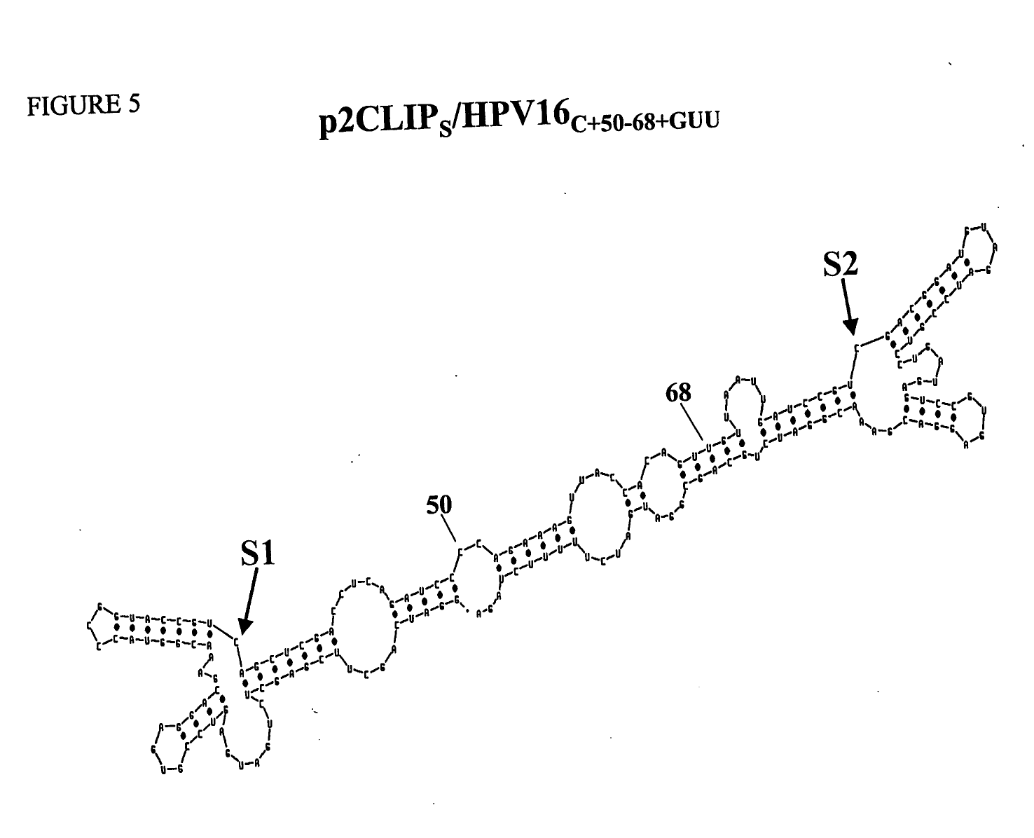

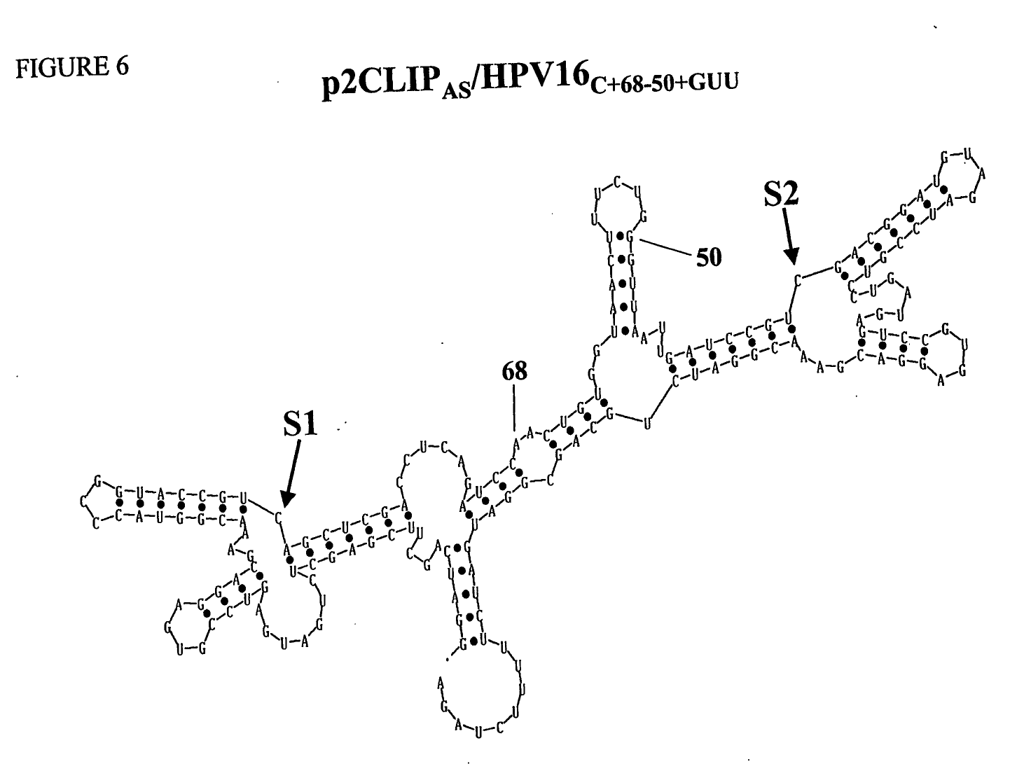

Two p2CLIP cassettes are constructed to contain one strand that is a template for (1) a cis-acting ribozyme sequence followed by (2) either a sense or antisense sequence followed by (3) a cis acting ribozyme sequence (p2CLIP_{S}/HPV16_{C+50-68+GUU} and p2CLIP_{AS}/HPV16_{C+68-50+GUU}; FIGS. 5 and 6).

FIGS. 5 and 6 contain the predicted structure (mFold program; see, e.g., Zuker and Jacobson, RNA, 4:669-679 (1998)) of the RNA molecules transcribed from p2CLIP_{S}/HPV16_{C+50-68+GUU} and p2CLIP_{AS}/HPV16_{C+68-50+GUU}, respectively. A portion of p2CLIP_{S}/HPV16_{C+50-68+GUU} contains the following sequence: 5′-GGATCAGCTTCGAGCTCTGATGAGTCCGTGAGGACGAAACGGTACCCGGTACCGTCAGCTCGACCTCAGATCCCCAGAAAGTTACCACAGTTGTTAATTGATCCGTCGACGGATGTAGATCCGTCCTGATGAGTCCGTGAGGACGAAACGGATCTGCAGCGGATGATCTTTTCTAGA-3′ (SEQ ID NO: 16). The underlined sequence corresponds with the sense sequence, and the bold nucleotide is the nucleotide present in the HPV strain having its sequence set forth in GenBank® Accession No. gi333031. To correspond to a Hershey HPV strain sequence, the “A” can be replaced with a “G.” In this case, the cassette would be designated p2CLIP_{S}/HPV16_{C+50-68(H)GUU}.

A portion of p2CLIP_{AS}/HPV16_{C+68-50+GUU} contains the following sequence: 5′-GGATCAGCTTCGAGCTCTGATGAGTCCGTGAGGACGAAACGGTACCCGGTACCGTCAGCTCGACCTCAGATCCAACTGTGGTAACTTTCTGGGTTAATTGATCCGTCGACGGATGTAGATCCGTCCTGATGAGTCCGTGAGGACGAAACGGATCTGCAGCGGATGATCTTTTTCTAGA-3′ (SEQ ED NO: 17). The underlined sequence corresponds with the antisense sequence, and the bold nucleotide is the nucleotide from GenBank® Accession No. gi333031. To correspond to a Hershey HPV strain sequence, the “T” can be replaced with a “C.” In this case, the cassette would be designated p2CLIP_{AS}/HPV16_{C+68-50(H)+GUU}.

To construct the p1CLIP cassettes, portions of the constructs were synthesized as overlapping oligonucleotides that were combined together to make the p1CLIP cassettes using standard cloning vectors such as pCRII vector. To construct the p2CLIP cassettes, the various HPV regions like HPV16_{C+50-68+GUU} are synthesized as overlapping oligonucleotides with the appropriate endonuclease restriction sites on the ends. The oligonucleotides are then digested and ligated into the digested p2CLIP cassette. In each case, common molecular cloning techniques similar to those described elsewhere were or are used (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RNA, 7:610-621 (2001); Zhang et al., Exp. Cell Res., 273:73-84 (2002); and Pan et al., Mol. Ther., 7:129-139 (2003)).

### Example 2

### Construction of pCLIP Cassettes Containing Hepatitis B Virus (HBV) Sequences

Two p1CLIP cassettes were constructed to contain one strand that is a template for (1) a cis-acting ribozyme sequence followed by (2) either a sense or antisense sequence followed by (3) a cis acting ribozyme sequence (P1CLIP_{S}/HBV₈₆₂₋₈₈₂ and p1CLIP_{AS}/HBV₈₈₂₋₈₆₂). The HBV₈₆₂₋₈₈₂ sequence is 5′-TTTGGAGCTACTGT-GGAGTTA-3′ (SEQ ID NO: 18). The HBV sequences used herein correspond to the HBV sequences found in GenBank® Accession No. X02496.

A portion of p1CLIP_{S}/HBV₈₆₂₋₈₈₂ contains the following sequence: 5′-AAGCTTCTCCAAACTGATGAGTCCAATTGGACGAAACGGTACTCGAGTACCGTCTTTGGAGCTACTGTGGAGTTATTGACGGAGAATTCTCCGTCCTGATGAGTCCGGCCGGACGAAATAACTCAGATCTTTTTCTAGA-3′ (SEQ ID NO: 19). A portion of p1 CLIP_{AS}/HBV₈₈₂₋₈₆₂ contains the following sequence: 5′-AAGCTTGGAGTTACTGATGAGTCCAATTGGACGAAACGGTACTCGAGTACCGTCTAACTCCACAGTAGCTCCAAATTGACGGAGAATTCTCCGTCCTGATGAGTCCGGCCGGACGAAATTTGGAAGATCTTTTTCTAGA-3′ (SEQ ID NO: 20). In each case, the middle underlined sequence is either the sense or antisense sequence that corresponds to an HBV sequence. The first underlined sequence is complementary to a first region of the sense or antisense sequence and helps position the cis-acting ribozyme cleavage site within a few nucleotides of the sense or antisense sequence. The last underlined sequence is complementary to a last region of the sense or antisense sequence and helps position the cis-acting ribozyme cleavage site within a few nucleotides of the sense or antisense sequence. In some cases, the cassette can be designed such that the sense and antisense sequences are further away from the cleavage sites.

To construct the p1CLIP cassettes, portions of the constructs were synthesized as overlapping oligonucleotides that were combined together to make the p1CLIP cassettes using standard cloning vectors such as pCRII vector. In addition, common molecular cloning techniques similar to those described elsewhere were used (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RNA, 7:610-621 (2001); Zhang et al., Exp. Cell Res., 273:73-84 (2002); and Pan et al., Mol. Ther., 7:129-139 (2003)).

### Example 3

### Construction of pCHOP Cassettes

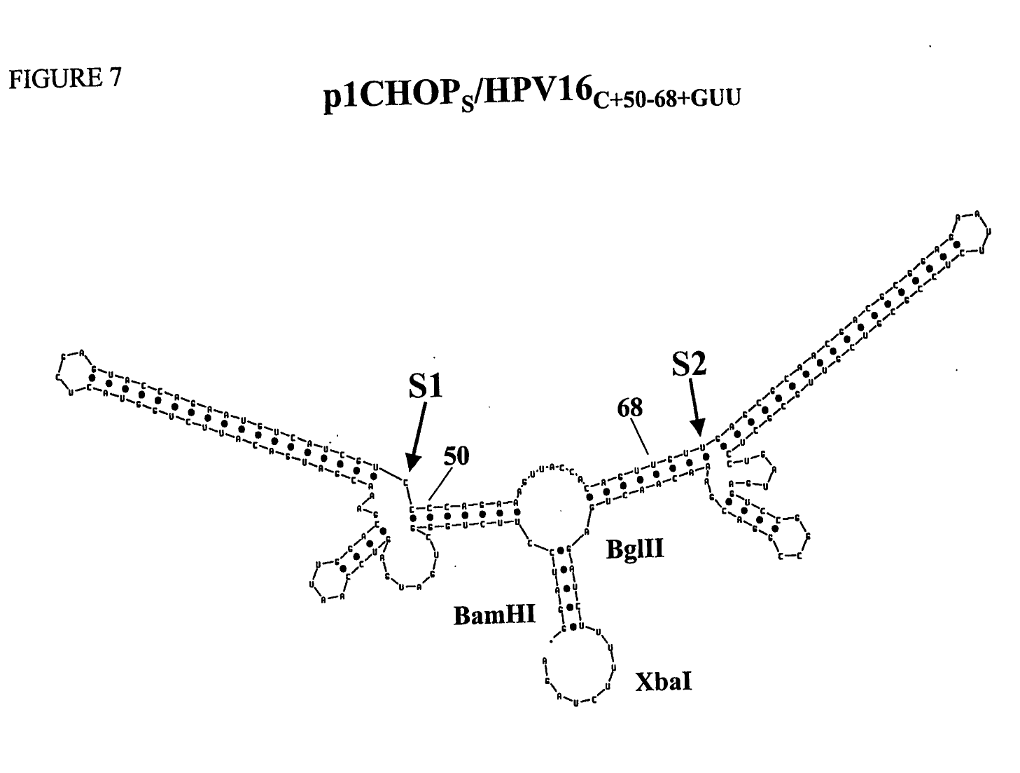

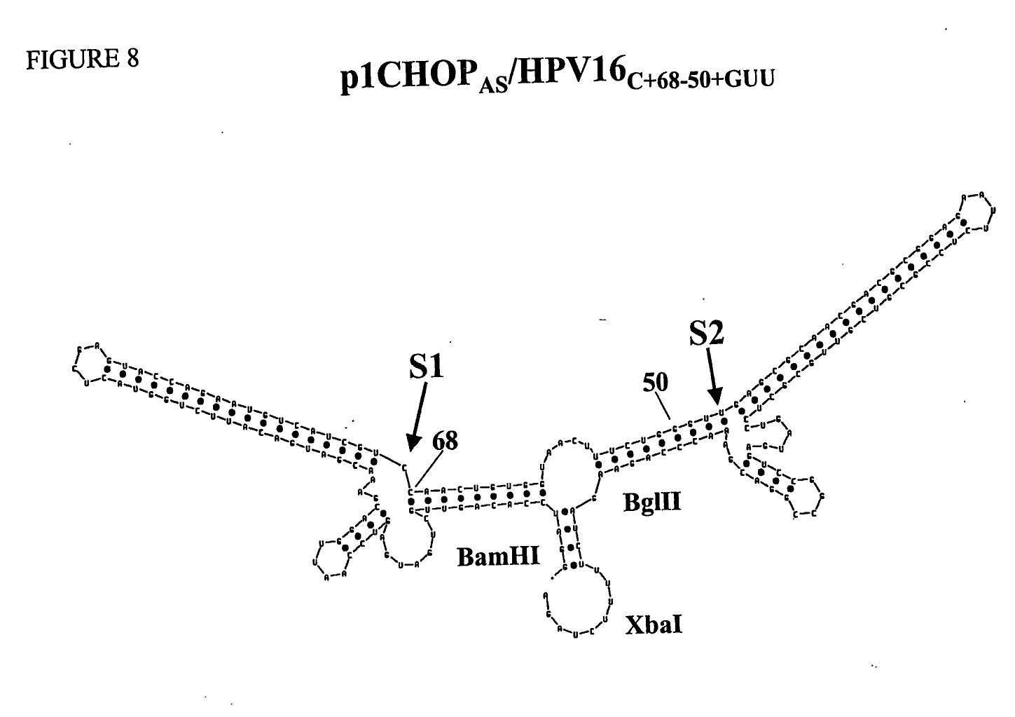

Four p1CHOP cassettes were constructed to contain one strand that is a template for (1) a cis-acting ribozyme sequence followed by (2) either a sense or antisense sequence followed by (3) a cis acting ribozyme sequence (p1CHOP_{S}/HPV16_{C+50-68(H)GUU}; p1CHOP_{AS}/HPV16_{C+68-50(H)+GUU}; p1CHOP_{S}/HPV16₅₇₋₇₇; and p1CHOP_{AS}/HPV16₇₇₋₅₇). FIGS. 7 and 8 contain the predicted structure (in Fold program; see, e.g., Zuker and Jacobson, RNA, 4:669-679 (1998)) of the RNA molecules transcribed from p1CHOP_{S}/HPV16_{C+50-68+GUU} and p1CHOP_{AS}/HPV16_{C+68-50+GUU}, respectively. A portion of p1CHOP_{S}/HPV16_{C+50-68+GUU} contains the following sequence: 5′-GGATCCTTCTGGGCTGATGAGTCCAATTGGACGAAACGATGACATTCTGGTACTCGAGTACCAGAATGTCATCGTCCCCAGAAAGTTACCACAGTTGTTGAGCGCAACGACGCGGAGAATTCTCCGCGTCGTTGCGCTCCTGATGAGTCCGGCCGGACGAAACAACTGAGATCTTTTTCTAGA-3′ (SEQ D NO: 21). To correspond to a Hershey HPV strain sequence, the bold “T” was replaced with a “C,” and the bold “A” was replaced with a “G.” In this case, the cassette was designated p1CHOP_{S}/HPV16_{C+50-68(H)+GUU}. A portion of p1CHOP_{AS}/HPV16_{C+68-50+GUU} contains the following sequence: 5′-GGATCCACAGTTGCTGATGAGTCCAATTGGACGAAACGATGACATTCTGGTACTCGAGTACCAGAATGTCATCGTCCAACTGTGGTAACTTTCTGGGTTGAGCGCAACGACGCGGAGAATTCTCCGCGTCGTTGCGCTCCTGATGAGTCCGGCCGGACGAAACCCAGAAGATCTTTTTCTAGA-3′ (SEQ ID NO: 22). To correspond to a Hershey HPV strain sequence, the bold “T” was replaced with a “C,” and the bold “A” was replaced with a “G.” In this case, the cassette was designated p1CHOP_{AS}/HPV16_{C+68-50(H)+GUU}.

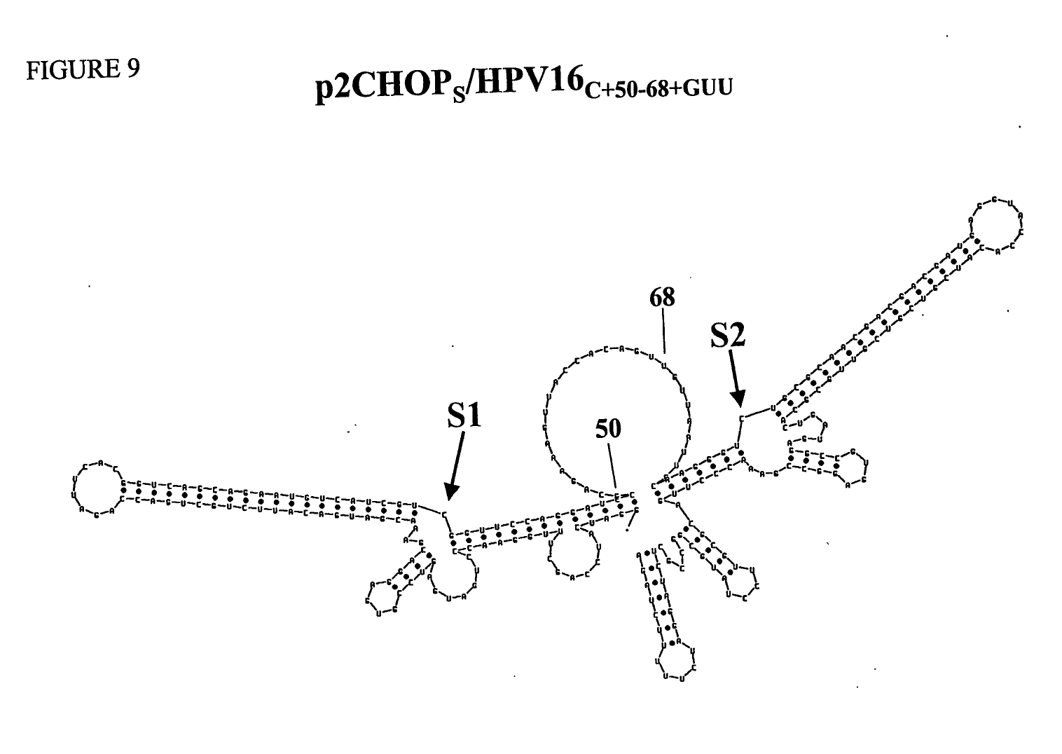

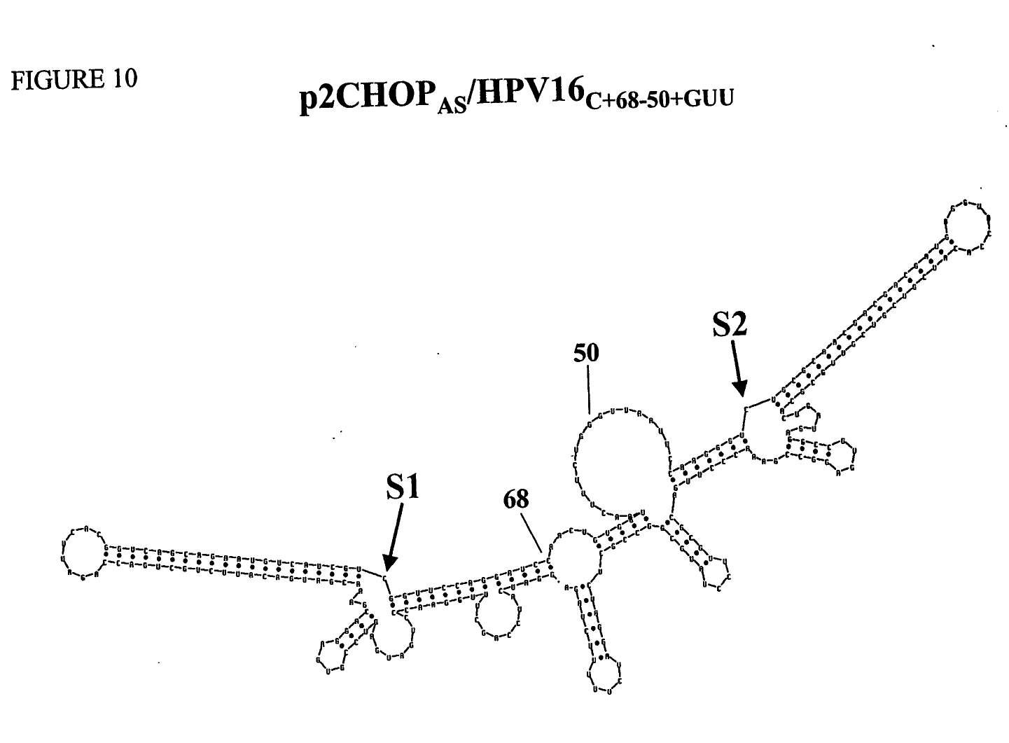

Two p2CHOP cassettes are constructed to contain one strand that is a template for (1) a cis-acting ribozyme sequence followed by (2) either a sense or antisense sequence followed by (3) a cis acting ribozyme sequence (p2CHOP_{S}/HPV16_{C+50-68+GUU} and p2CHOP_{AS}/HPV16_{C+68-50+GUU}; FIGS. 9 and 10). FIGS. 9 and 10 contain the predicted structure (mFold program; see, e.g., Zuker and Jacobson, RNA, 4:669-679 (1998)) of the RNA molecules transcribed from p2CHOP_{S}/HPV16_{C+50-68+GUU} and p2CHOP_{AS}/HPV16_{C+68-50+GUU}, respectively. A portion of p2CHOP_{S}/HPV16_{C+50-68+GUU} contains the following sequence: 5′-GGATCATCCAGCTTTGGAACCCTGATGAGTCCGTGAGGACGAAACGATGACATTCTGCTGACCAGATTCACGGTCAGCAGAATGTCATCGTCGGTTCCAGGATCCCCAGAAAGTTACCACAGTTGTTAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGGATCTTTTTCTAGA-3′ (SEQ ID NO: 23). To correspond to a Hershey HPV strain sequence, the bold “A” can be replaced with a “G.” In this case, the cassette would be designated p2CHOP_{S}/HPV16_{C+50-68(H)+GUU}. A portion of p2CHOP_{AS}/HPV16_{C+68-50+GUU} contains the following sequence: 5′-GGATCATCCAGCTTTGGAACCCTGATGAGTCCGTGAGGACGAAACGATGACATTCTGCTGACCAGATTCACGGTCAGCAGAATGTCATCGTCGGTTCCAGGATCCAACTGTGGTAACTTTCTGGGTTAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGGATCTTTTTCTAGA-3′ (SEQ ID NO: 24). To correspond to a Hershey HPV strain sequence, the bold “T” can be replaced with a “C.” In this case, the cassette would be designated p2CHOP_{AS}/HPV16_{C+68-50(H)+GUU}.

To construct the p1CHOP cassettes, portions of the constructs were synthesized as overlapping oligonucleotides that were combined together to make the p1CHOP cassettes using standard cloning vectors such as pCRII vector. To construct the p2CHOP cassettes, the various HPV regions like HPV16_{C+50-68+GUU} are synthesized as overlapping oligonucleotides with the appropriate endonuclease restriction sites on the ends. The oligonucleotides are then digested and ligated into the digested p2CHOP cassette. In each case, common molecular cloning techniques similar to those described elsewhere were or are used (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RNA, 7:610-621 (2001); Zhang et al., Exp. Cell Res., 273:73-84 (2002); and Pan et al., Mol. Ther., 7:129-139 (2003)).

### Example 4

### Construction of pSIR Cassettes from pCLIP and pCHOP Cassettes

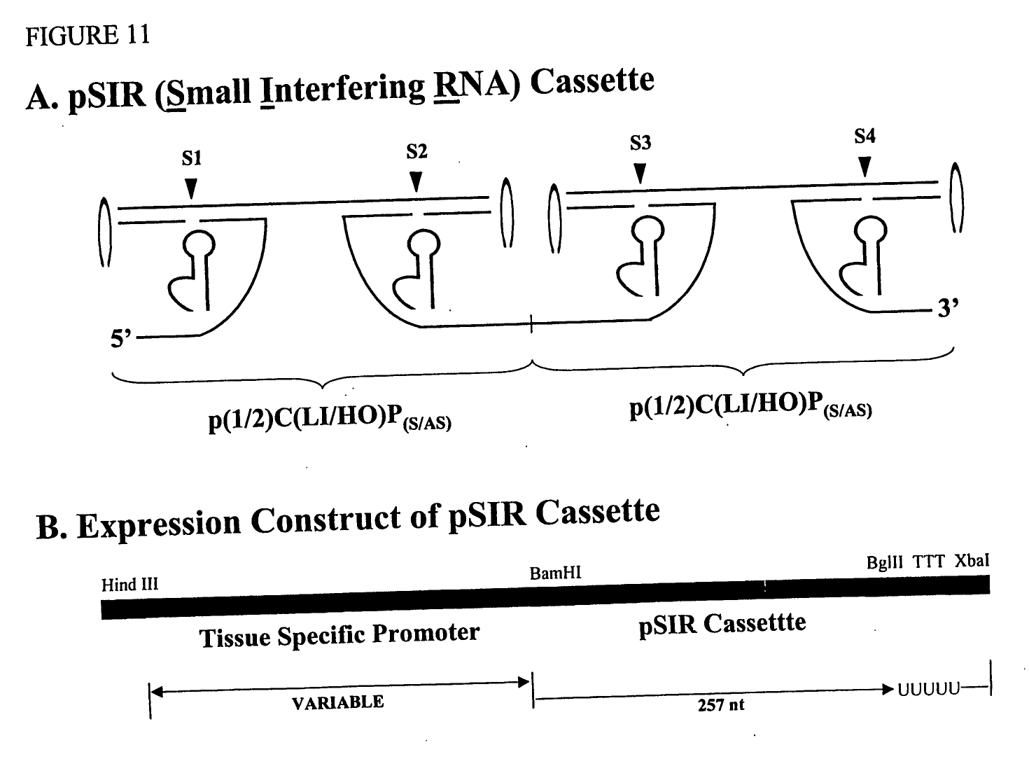

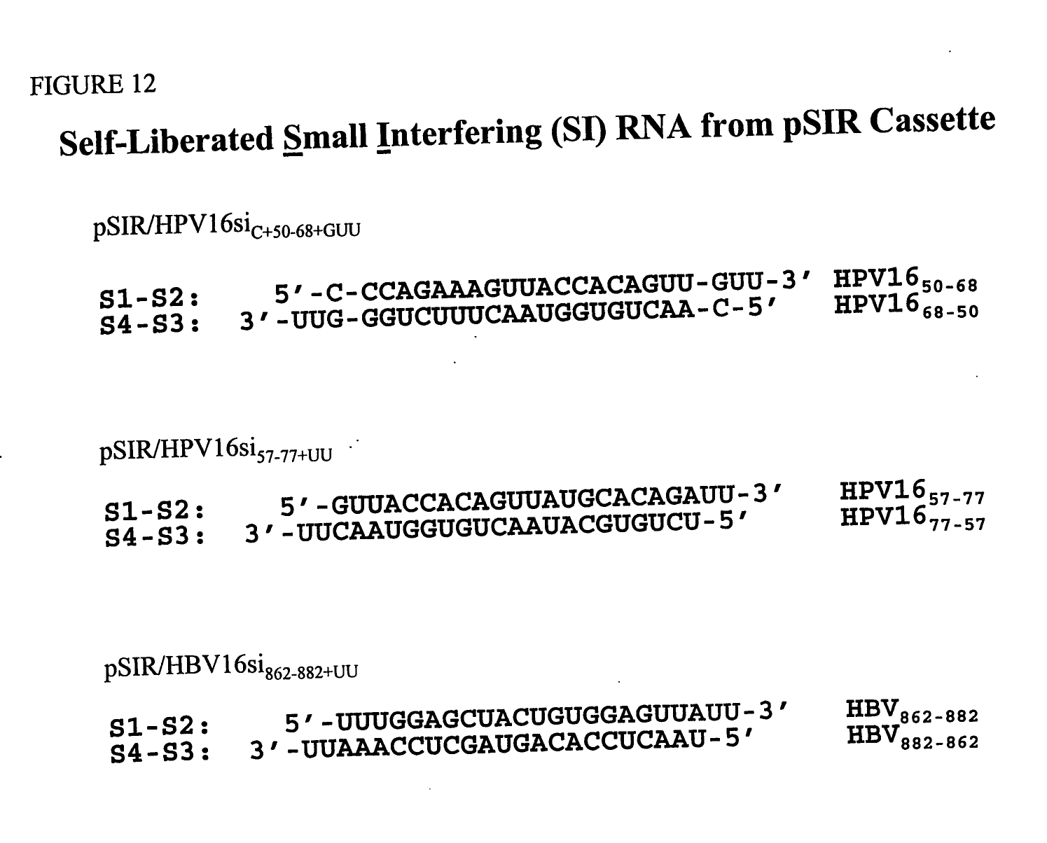

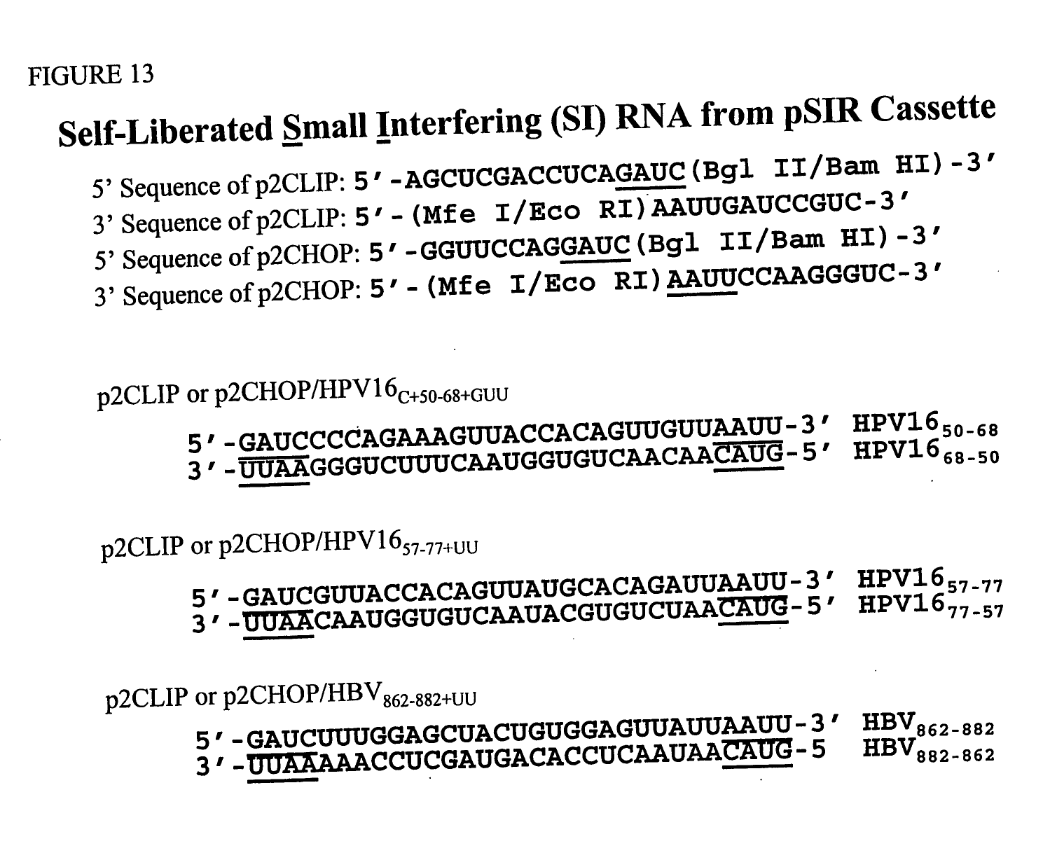

The p1CLIP_{S} and p1CLIP_{AS} cassettes were used to construct pSIR cassettes containing one strand that is a template for (1) a cis-acting ribozyme sequence followed by (2) a sense sequence followed by (3) a cis acting ribozyme sequence followed by (4) a cis-acting ribozyme sequence followed by (5) an antisense sequence followed by (6) a cis acting ribozyme sequence (FIG. 11). Similar pSIR cassettes can be made using any combination of two or more of the following cassettes: p1CLIP_{S}, p1CLIP_{AS}, p2CLIP_{S}, p2CLIP_{AS}, p1CHOP_{S}, p1CHOP_{AS}, p2CHOP_{S}, and p2CHOP_{AS}. Specifically, the p1CLIP_{S}/HPV16_{C+50-68(H)+GUU} and p1CLIP_{AS}/HPV16_{C+68-50(H)-GUU} cassettes were used to construct pSIR/HPV16si_{C+50-68(H)GUU}; the p1CLIP_{S}/HPV16_{57-77+UU} and pCLIP_{AS}/HPV16_{77-57+UU} cassettes were used to construct pSIR/HPV16si_{57-77+UU}; and the p1CLIP_{S}/HBV₈₆₂₋₈₈₂ and p1CLIP_{AS}/HBV₈₈₂₋₈₆₂ cassettes were used to construct pSIR/HBVsi₈₆₂₋₈₈₂. The RNA liberated from an RNA molecule transcribed from a pSIR cassette can form double-stranded RNA capable of inducing RNA interference (FIGS. 12 and 13).

A portion of pSIR/HPV16si_{C+50-68(H)+GUU} contains the following sequence: 5′-GGATCCTTCCGGGCTGATGAGTCCAATTGGACGAAACGGTACTCGAGTACCGTCCCCGGAAAGTTACCACAGTTGTTGACGGAGAATTCTCCGTCCTGATGAGTCCGGCCGGACGAAACAACTGAGATCCACAGTTGCTGATGAGTCCAATTGGACGAAACGGTACTCGAGTACCGTCCAACTGTGGTAACTTTCCGGGTTGACGGAGAATTCTCCGTCCTGATGAGTCCGGCCGGACGAAACCCGGAAGATCTTTTTCTAGA-3′ (SEQ ID NO: 25). A portion of pSIR/HPV16si_{57-77+UU} contains the following sequence: 5′-GGATCCTGGTAACGCTGATGAGTCCAATTGGACGAAACGGTACTCGAGTACCGTCCGTTACCACAGTTATGCACAGAGTTGACGGAGAATTCTCCGTCCTGATGAGTCCGGCCGGACGAAATCTGTGAGATCCGCACAGAGCTGATGAGTCCAATTGGACGAAACGGTACTCGAGTACCGTCCTCTGTGCATAACTGTGGTAACGTTGACGGAGAATTCTCCGTCCTGATGAGTCCGGCCGGACGAAACGTTACCAGATCTTTTTCTAGA-3′ (SEQ ID NO: 26). A portion of pSIR/HBVsi₈₆₂₋₈₈₂ contains the following sequence: 5′-AAGCTTCTCCAAACTGATGAGTCCAATTGGACGAAACGGTACTCGAGTACCGTCTTTGGAGCTACTGTGGAGTTATTGACGGAGAATTCTCCGTCCTGATGAGTCCGGCCGGACGAAATAACTCATGGAGTTACTGATGAGTCCAATTGGACGAAACGGTACTCGAGTACCGTCTAACTCCACAGTAGCTCCAAATTGACGGAGAATTCTCCGTCCTGATGAGTCCGGCCGGACGAAATTTGGAAGATCTTTTTCTAGA-3′ (SEQ ID NO: 27).

Expression of the pSIR cassettes containing HPV sequences was driven by the following U6 promoter sequence: 5-AAGGTCGGGCAGGAAGAGGGCCTATTTCCCATGATTCCTTCATATTTGCATATACGATACAAGGCTGTTAGAGAGATAATTAGAATTAATTTGACTGTAAACACAAAGATATTAGTACAAAATACGTGACGTAGAAAGTAATAATTTCTTGGGTAGTTTGCAGTTTTTAAAATTATGTTTTAAAATGGACTATCATATGCTTACCGTAACTTGAAAGTATTTCGATTTCTTGGCTTTATATATCTTGTGGAAAGGACGAAACACC-3′ (SEQ ED NO: 28; FIG. 11).

Expression of the pSIR cassettes containing HBV sequences was driven by the following albumin enhancer/promotor sequence: 5′-AGCTTGCAAGCATAGCACAGAGCAATGTTCTACTTTAATTACTTTCATTTTCTTGTATCCTCACAGCCTAGAAAATAACCTGCGTTACAGCATCCACTCAGTATCCCTTGAGCATGAGGTGACACTACTTAACATAGGGACGAGATGGTACTTTGTGTCTCCTGCTCTGTCAGCAGGGCACTGTACTTGCTGATACCAGGGAATGTTTGTTCTTAAATACCATCATTCCGGACGTGTTTGCCTTGGCCAGTTTTCCATGTACATGCAGAAAGAAGTTTGGACTGATCAATACAGTCCTCTGCCTTTAAAGCAATAGGAAAAGGCCAACTTGTCTACGAGTCGACGGATCCGGGCTCAAATGGGAGACAAAGAGATTAAGCTCTTATGTAAAATTTGCTGTTTTACATAACTTTAATGAATGGACAAAGTCTTGTGCATGGGGGTGGGGGTGGGGTTAGAGGGGAACAGCTCCAGATGGCAAACATACGCAAGGGATTTAGTCAAACAACTTTTTGGCAAAGATGGTATGATTTTGTAATGGGGTAGGAACCAATGAAATGCGAGGTAAGTATGGTTAATGATCTACAGTTATTGGTTAAAGAAGTATATTAGAGCGAGTCTTTCTGCACACAGATCACCTTTCCTATCAACCCCCCGGATCTCGAAGCTT-3′ (SEQ ID NO: 29; FIG. 11). The underlined sequence is a HindIII restriction enzyme site.

In each case, common molecular cloning techniques similar to those described elsewhere were used to make the pSIR cassettes (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RNA, 7:610-621 (2001); Zhang et al., Exp. Cell Res., 273:73-84 (2002); and Pan et al., Mol. Ther., 7:129-139 (2003)).

### Example 5

### Construction of pCLIP and pCHOP Cassettes with Three Cis-Acting Ribozyme Sequences (pCLIP_{HR} and pCHOP_{HR})

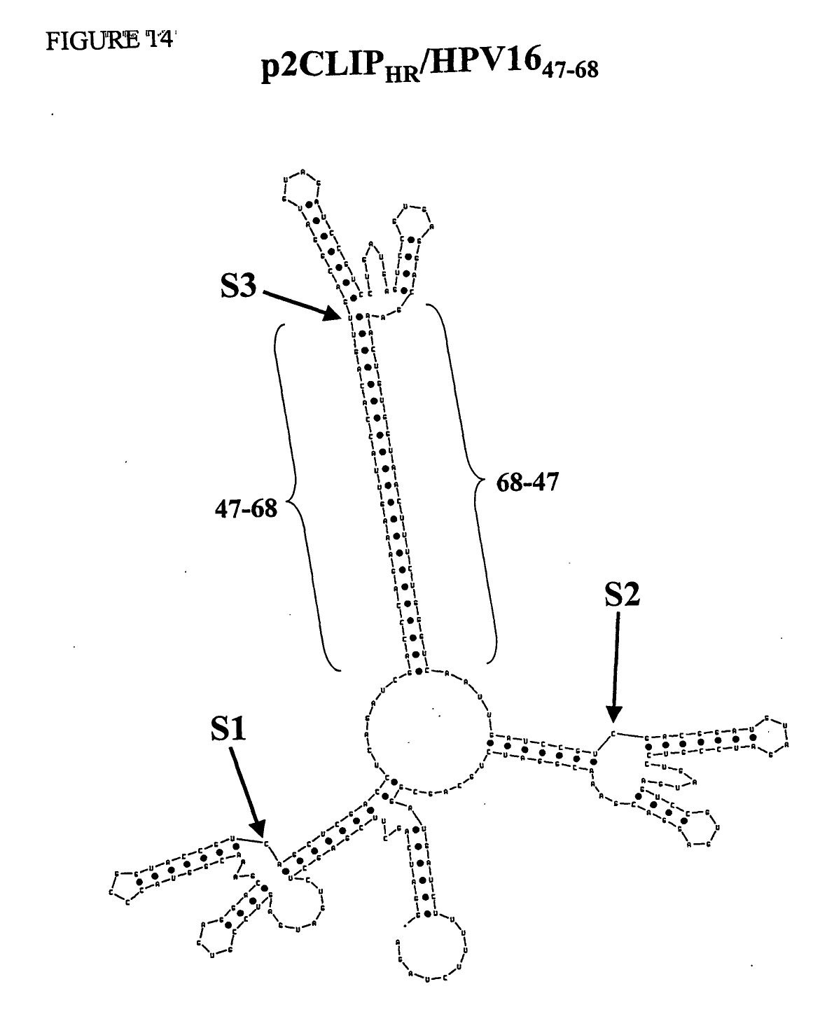

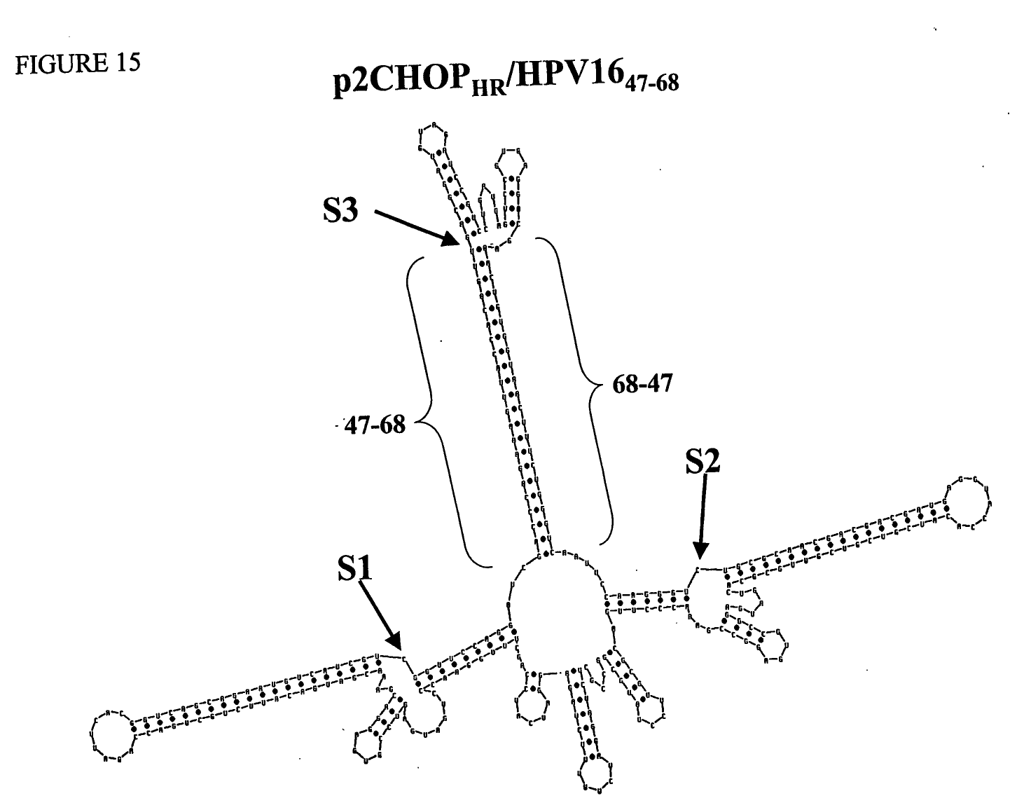

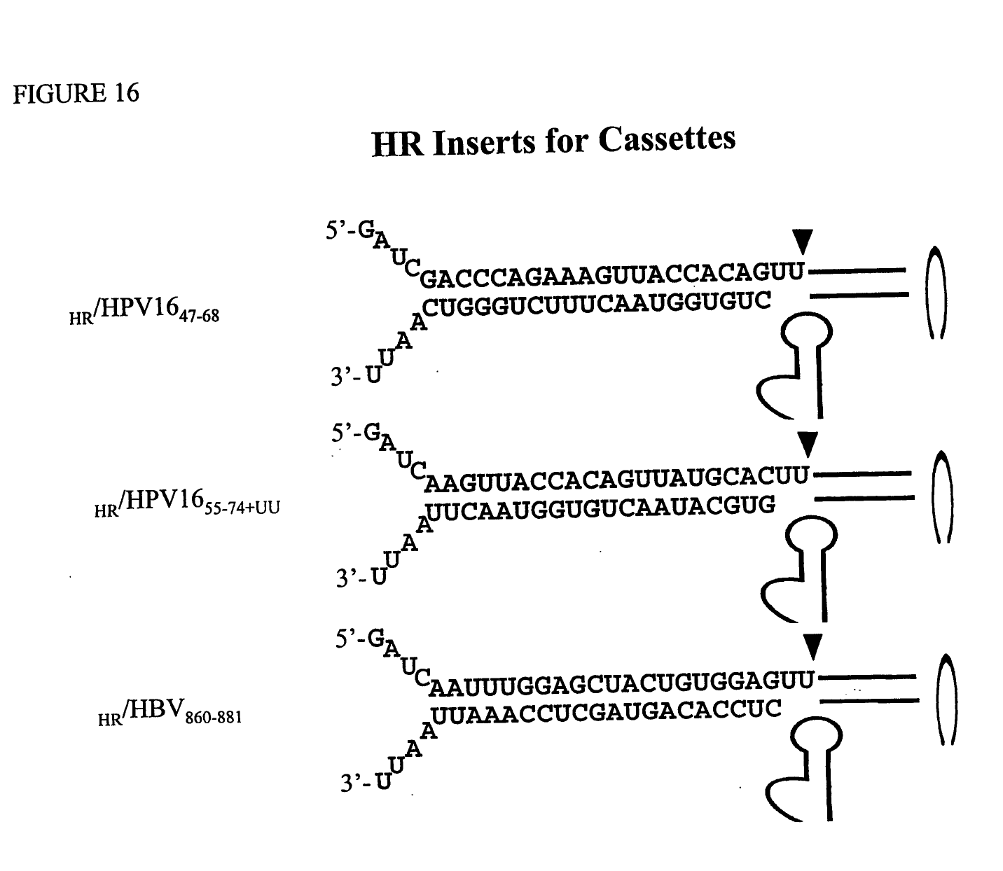

p2CLIP and p2CHOP cassettes were constructed to contain one strand that is a template for (1) a cis-acting ribozyme sequence followed by (2) a sense sequence followed by (3) a cis acting ribozyme sequence followed by (4) an antisense sequence followed by (5) a cis-acting ribozyme sequence (p2CLIP_{HR}/HPV16₄₇₋₆₈ (FIG. 14); p2CHOP_{HR}/HPV16₄₇₋₆₈ FIG. 15); p2CLIP_{HR}/HPV16_{55-74+UU}; and p2CHOP_{HR}/HPV16_{55-74+UU}). The RNA transcribed from these cassettes can form double-stranded RNA capable of inducing RNA interference (FIG. 16).

A portion of p2CLIP_{HR}/HPV16₄₇₋₆₈ contains the following sequence: 5′-GGATCAGCTTCGAGCTCTGATGAGTCCGTGAGGACGAAACGGTACCCGGTACCGT CAGCTCGACCTCAGATCGACCCAGAAAGTTACCACAGTTGACGGATGTAGATCCGTCCTGATGAGTCCGTGAGGACGAAACTGTGGTAACTTTCTGGGTCAATTGATCCGTCGACGGATGTAGATCCGTCCTGATGAGTCCGTGAGGACGAAACGGATCTGCAGCGGATGATCTITTTCTAGA-3′ (SEQ ID NO: 30). A portion of p2CHOP_{HR}/HPV16₄₇₋₆₈ contains the following sequence: 5′-GGATCATCCAGCTTTGGAACCCTGATGAGTCCGTGAGGACGAAACGATGACATTCTGCTGACCAGATTCACGGTCAGCAGAATGTCATCGTCGGTTCCAGGATCGACCCAGAAAGTTACCACAGTTGACGGATGTAGATCCGTCCTGATGAGTCCGTGAGGACGAAACTGTGGTAACTTTCTGGGTCAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGGATCTTTTTCTAGA-3′ (SEQ ID NO: 31). A portion of p2CLIP_{HR}/HPV16_{55-74+UU} contains the following sequence: 5′-GGATCAGCTTCGAGCTCTGATGAGTCCGTGAGGACGAAACGGTACCCGGTACCGTCAGCTCGACCTCAGATCAAGTTACCACAGTTATGCACTTGACGGATGTAGATCCGTCCTGATGAGTCCGTGAGGACGAAAGTGCATAACTGTGGTAACTTAATTGATCCGTCGACGGATGTAGATCCGTCCTGATGAGTCCGTGAGGACGAAACGGATCTGCAGCGGATGATCTTTTTCTAGA-3′ (SEQ ID NO:32). A portion of p2CHOP_{HR}/HPV16_{55-74+UU} contains the following sequence: 5′-GGATCATCCAGCTTTGGAACCCTGATGAGTCCGTGAGGACGAAACGATGACATTCTGCTGACCAGATTCACGGTCAGCAGAATGTCATCGTCGGTTCCAGGATCAAGTTACCACAGTTATGCACTTGACGGATGTAGATCCGTCCTGATGAGTCCGTGAGGACGAAAGTGCATAACTGTGGTAACTTAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGGATCTTTTTCTAGA-3′ (SEQ ID NO:33). In each case, the sense and antisense sequences are underlined.

To construct the p2CLIP_{HR} and p2CHOP_{HR} cassettes, the various HPV regions like HPV16₄₇₋₆₈ were synthesized as overlapping oligonucleotides with the appropriate endonuclease restriction sites on the ends. The oligonucleotides were then digested and ligated into the digested p2CLIP or p2CHOP cassette. In each case, common molecular cloning techniques similar to those described elsewhere were used (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RNA, 7:610-621 (2001); Zhang et al., Exp. Cell Res., 273:73-84 (2002); and Pan et al., Mol. Ther., 7:129-139 (2003)).

### Example 6

### Construction of pSNIP Cassettes

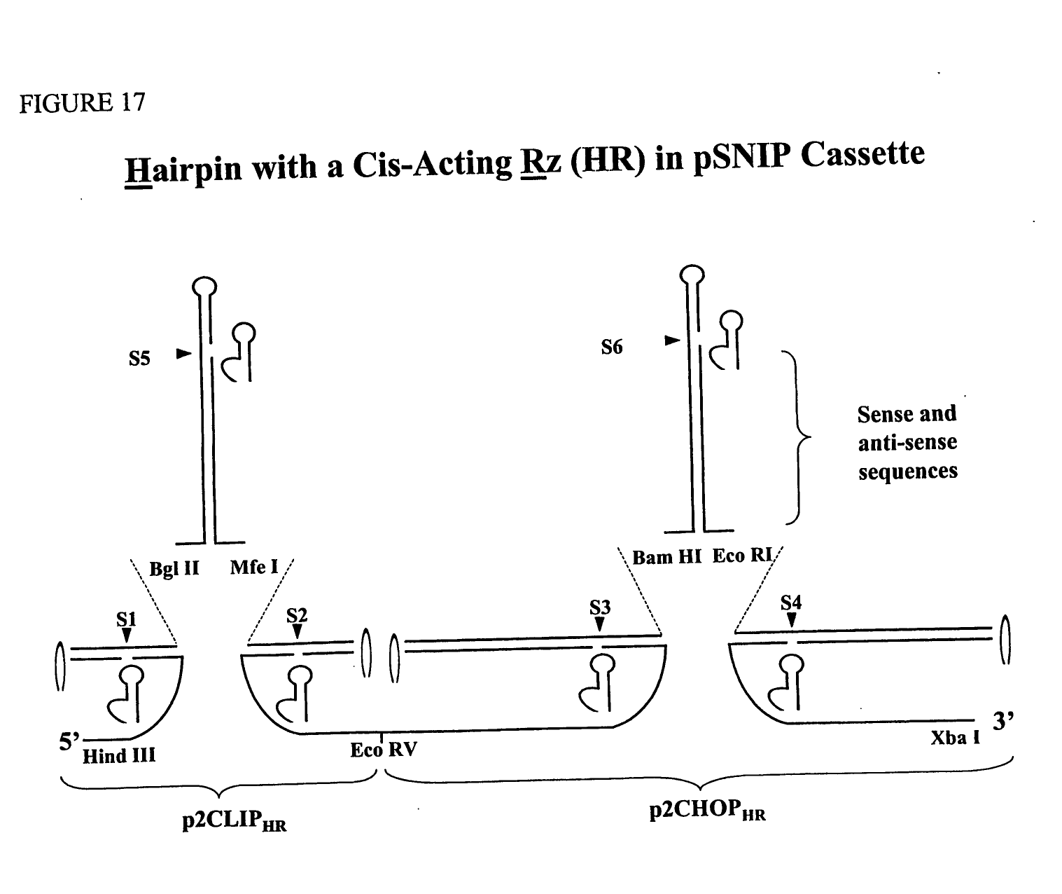

pCLIP_{HR} cassettes (p1CLIP_{HR} or p2CLIP_{HR}) and pCHOP_{HR} cassettes (p1CHOP_{HR} or p2CHOP_{HR}) or combinations thereof were combined to construct pSNIP cassettes containing one strand that is a template for (1) a cis-acting ribozyme sequence followed by (2) a sense sequence followed by (3) a cis acting ribozyme sequence followed by (4) an antisense sequence followed by (5) a cis-acting ribozyme sequence followed by (6) a cis-acting ribozyme sequence followed by (7) a sense sequence followed by (8) a cis acting ribozyme sequence followed by (9) an antisense sequence followed by (10) a cis-acting ribozyme sequence FIG. 17). The pSNIP cassette can have the following general sequence: 5′-AAGCTT(HindIII)CGAGCTCTGATGAGTCCGTGAGGACGAAACGGTACCCGGTACCGTCAGCTCGACCTCAGATCT(BglII)CTCGAGCAATTG(MfeI)ATCCGTCGACGGATGTAGATCCGTCCTGATGAGTCCGTGAGGACGAAACGGATCTGCAGCGGATATCCAGCTTTGGAACCCTGATGAGTCCGTGAGGACGAAACGATGACATTCTGCTGACCAGATTCACGGTCAGCAGAATGTCATCGTCGGTTCCAGGATCC(BamHI)TTGCCTGAATTC(EcoRI)CAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGA(XbaI)-3′ (SEQ ID NO:34).

Specifically, the p2CLIP_{HR}/HPV16₄₇₋₆₈ and p2CHOP_{HR}/HPV16₄₇₋₆₈ cassettes were used to form pSNIP/HPV16₄₇₋₆₈, which contains the following sequence: 5′-GGATCAGCTTCGAGCTCTGATGAGTCCGTGAGGACGAAACGGTACCCGGTACCGTCAGCTCGACCTCAGATCGACCCAGAAAGTTACCACAGTTGACGGATGTAGATCCGTCCTGATGAGTCCGTGAGGACGAAACTGTGGTAACTTTCTGGGTCAATTGATCCGTCGACGGATGTAGATCCGTCCTGATGAGTCCGTGAGGACGAAACGGATCTGCAGCGGATATCCAGCTTTGGAACCCTGATGAGTCCGTGAGGACGAAACGATGACATTCTGCTGACCAGATTCACGGTCAGCAGAATGTCATCGTCGGTTCCAGGATCGACCCAGAAAGTTACCACAGTTGACGGATGTAGATCCGTCCTGATGAGTCCGTGAGGACGAAACTGTGGTAACTTTCTGGGTCAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGGATCTTTTTCTAGA-3′ (SEQ ID NO: 35).

The p2CLIP_{HR}/HPV16_{55-74+UU} and p2CHOP_{HR}/HPV16_{55-74+UU} cassettes were used to form pSNIP/HPV16_{55-74+UU}, which contains the following sequence: 5′-GGATCAGCTTCGAGCTCTGATGAGTCCGTGAGGACGAAACGGTACCCGGTACCGTCAGCTCGACCTCAGATCAAGTTACCACAGTTATGCACTTGACGGATGTAGATCCGTCCTGATGAGTCCGTGAGGACGAAAGTGCATAACTGTGGTAACTTAATTGATCCGTCGACGGATGTAGATCCGTCCTGATGAGTCCGTGAGGACGAAACGGATCTGCAGCGGATATCCAGCTTTGGAACCCTGATGAGTCCGTGAGGACGAAACGATGACATTCTGCTGACCAGATTCACGGTCAGCAGAATGTCATCGTCGGTTCCAGGATCAAGTTACCACAGTTATGCACTTGACGGATGTAGATCCGTCCTGATGAGTCCGTGAGGACGAAAGTGCATAACTGTGGTAACTTAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGGATCTTTTTCTAGA-3′ (SEQ ID NO: 36).

A pSNIP cassette was constructed to contain the following DNA fragment, which is a template for sense and antisense HBV sequences (HBV₈₆₀₋₈₈₁) flanking a cis-acting ribozyme sequence: 5′-GATCAATTTGGAGCTACTGTGGAGTTGACGGATGTAGATCCGTCCTGATGAGTCCGTGAGGACGAAACTCCACAGTAGCTCCAAATTAATT-3′ (SEQ ID NO: 37). A portion of this cassette (pSNIP/HBV₈₆₀₋₈₈₁) contains the following sequence: 5′-AAGCTTCGAGCTCTGATGAGTCCGTGAGGACGAAACGGTACCCGGTACCGTCAGCTCGACCTCAGATCAATTTGGAGCTACTGTGGAGTTGACGGATGTAGATCCGTCCTGATGAGTCCGTGAGGACGAAACTCCACAGTAGCTCCAAATTAATTGATCCGTCGACGGATGTAGATCCGTCCTGATGAGTCCGTGAGGACGAAACGGATCTGCAGCGGATATCCAGCTTTGGAACCCTGATGAGTCCGTGAGGACGAAACGATGACATTCTGCTGACCAGATTCACGGTCAGCAGAATGTCATCGTCGGTTCCAGGATCAATTTGGAGCTACTGTGGAGTTGACGGATGTAGATCCGTCCTGATGAGTCCGTGAGGACGAAACTCCACAGTAGCTCCAAATTAATTCCAAGGGTCTGCGCAACGACGACGATGAGGTACCACATCGTCGTCGTTGCGCACTGATGAGGCCGTGAGGCCGAAACCCTTGACGCGTTCCTATGCGGCCGCTCTAGA-3′ (SEQ ID NO: 38).

These cassettes were constructed using common molecular cloning techniques similar to those described elsewhere (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RIA, 7:610-621 (2001); Zhang et al., Exp. Cell Res., 273:73-84 (2002); and Pan et al., Mol. Ther., 7:129-139 (2003)).

### Example 7

### Construction of pCLIP and pCHOP Cassettes with Hairpin Loop Sequences

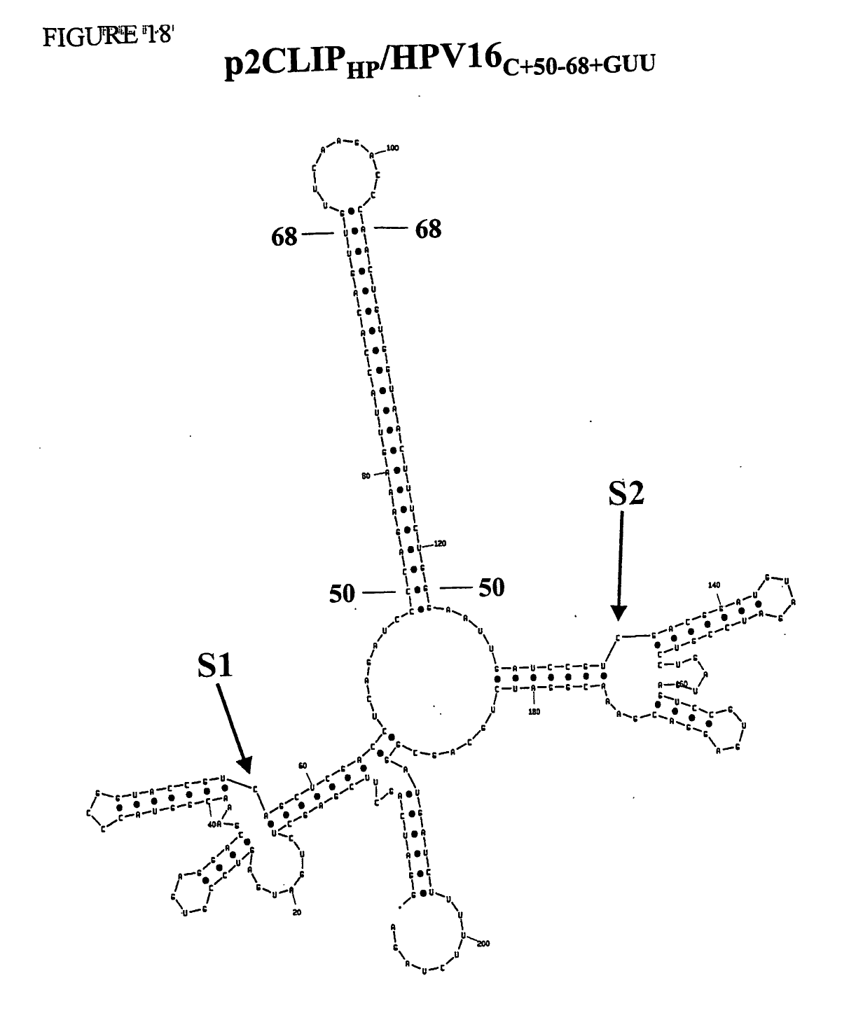

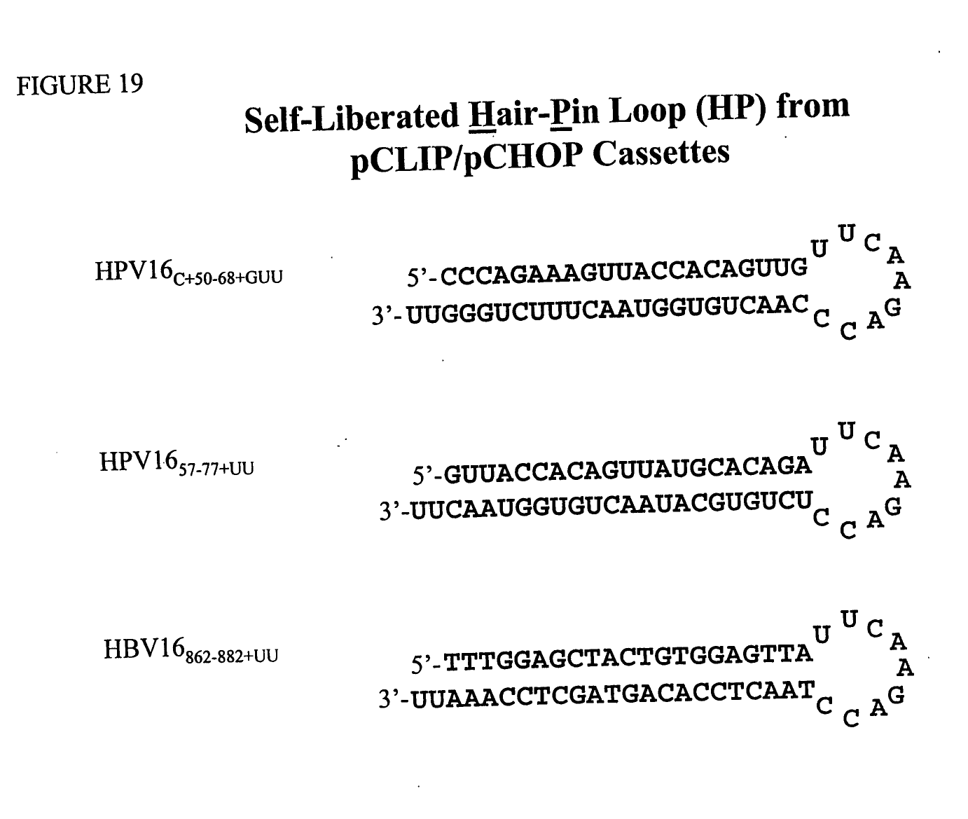

Three p1CLIP cassettes were constructed to contain one strand that is a template for (1) a cis-acting ribozyme sequence followed by (2) a hairpin loop sequence where the stem portion contains sense and antisense sequences followed by (3) a cis acting ribozyme sequence (p1CLIP_{HP}/HPV16_{C+50-68(H)+GUU}; p1CLIP_{HP}/HPV16₅₇₋₇₇; and p1CLIP_{HP}/HBV₈₆₂₋₈₈₂). In addition, a p2CLIP_{HP}/HPV16_{C+50-68(H)+GUU} construct was made. FIG. 18 contains the predicted structure (mFold program; see, e.g., Zuker and Jacobson, RNA, 4:669-679 (1998)) of the RNA molecules transcribed from p2CLIP_{HP}/HPV16_{C+50-68+GUU}. Similar cassettes can be made using pCHOP sequences. The RNA transcribed from pCLIP_{HP} and pCHOP_{HP} cassettes can form single-stranded RNA capable of forming a hairpin loop structure, which can induce RNA interference (FIG. 19).

A portion of p1CLIP_{HP}/HPV16_{C+50-68(H)GUU} contains the following sequence: 5′-GGATCCTTCCGGGCTGATGAGTCCAATTGGACGAAACGGTACTCGAGTACCGTCCCCGGAAAGTTACCACAGTTGTTCAAGACCCAACTGTGGTAACTTTCCGGGTTGACGGAGAATTCTCCGTCCTGATGAGTCCGGCCGGACGAAACCCGGAAGATCTTTTTCTAGA-3′ (SEQ ID NO: 39). A portion of p1CLIP_{HP}/HPV16₅₇₋₇₇ contains the following sequence: 5′-GGATCCTGGTAACCTGATGAGTCCAATTGGACGAAACGGTACTCGAGTACCGTCGTTACCACAGTTATGCACAGATTCAAGACCTCTGTGCATAACTGTGGTAACTTGACGGAGAATTCTCCGTCCTGATGAGTCCGGCCGGACGAAAGTTACCAGATCTTTTTCTAGA-3′ (SEQ ID NO: 40). A portion of p1CLIP_{HP}/HBV₈₆₂₋₈₈₂ contains the following sequence: 5′-AAGCTTCTCCAAACTGATGAGTCCAATTGGACGAAACGGTACTCGAGTACCGTCTTTGGAGCTACTGTGGAGTTATTCAAGACCTAACTCCACAGTAGCTCCAAATTGACGGAGAATTCTCCGTCCTGATGAGTCCGGCCGGACGAAATTTGGAAGATCTTTTTCTAGA-3′ (SEQ ID NO: 41).

Two p1CLIP_{HP}/HBV₈₆₂₋₈₈₂ cassettes were joined together to form a cassette containing one strand that is a template for (1) a cis-acting ribozyme sequence followed by (2) a hairpin loop sequence where the stem portion contains sense and antisense sequences followed by (3) a cis acting ribozyme sequence followed by (4) a cis-acting ribozyme sequence followed by (5) a hairpin loop sequence where the stem portion contains sense and antisense sequences followed by (6) a cis acting ribozyme sequence (p1CLIP_{HP}-double/HBV₈₆₂₋₈₈₂). A portion of p1CLIP_{HP}-double/HBV₈₆₂₋₈₈₂ contains the following sequence: 5′-AAGCTTCTCCAAACTGATGAGTCCAATTGGACGAAACGGTACTCGAGTACCGTCTTTGGAGCTACTGTGGAGTTATTCAAGACCTAACTCCACAGTAGCTCCAAATTGACGGAGAATTCTCCGTCCTGATGAGTCCGGCCGGACGAAATTTGGAATCTCCAAACTGATGAGTCCAATTGGACGAAACGGTACTCGAGTACCGTCTTTGGAGCTACTGTGGAGTTATTCAAGACCTAACTCCACAGTAGCTCCAAATTGACGGAGAATTCTCCGTCCTGATGAGTCCGGCCGGACGAAATTTGGAAGATCTTTTTCTAGA-3′ (SEQ ID NO: 42).

Each cassette was constructed using common molecular cloning techniques similar to those described herein and elsewhere (Benedict et al., Carcinogenesis, 19:1223-1230 (1998); Schalles et al., Gene Ther. Mol. Biol., 3:257-269 (1999); Crone et al., Hepatology, 29:1114-1123 (1999); Pan et al., RNA, 7:610-621 (2001); Zhang et al., Exp. Cell Res., 273:73-84 (2002); and Pan et al., Mol. Ther., 7:129-139 (2003)).

### Example 8

### In Vivo Screen

A modified version of the Flp-In system commercially available from Invitrogen is used in conjunction with human 293 embryonic kidney cells as follows (FIG. 20). An HPV16 E6/E7 target sequence is cloned between the HindIII and EcoRV restriction endonuclease sites of pcDNA5-FRT such that transcription of the HPV mRNA is under control of the CMV promoter. The various sequences to be tested (library sequences) flanked by two U6 promoter sequences such that each strand is transcribed are cloned into the SapI site of pcDNA5-FRT. These library sequences can be randomly generated sequences or sequences obtained from an in vitro screening process (FIG. 21). The DNA encoding a red fluorescent protein (RFP) is added to the vector, either upstream or downstream from the DNA encoding the HPV16 E6/E7 target. When the HPV16 E6/E7 target mRNA is effectively destroyed, loss of fluorescence occurs.

These constructs are then co-transfected together with pOG44 constructs (which produce the recombinase) into the Flp-In 293 cell line. These cells are resistant to Zeocin selection and express RFP. Selection of stably transfected cells is performed using 20 μg/mL of Hygromycin B. After recombination, the cells become sensitive to Zeocin and fluorescence is lost when the HPV16 E6/E7 target mRNA is degraded. After clonal expansion of the stably transfected cells that show reduced fluorescence, the identity of the sequence responsible for the degradation is determined using PCR-based sequencing.

An HPV16_{47-68(H)} sequence was placed between the U6 promoter sequences and tested for the ability to reduce fluorescence. In addition, an HPV16₅₇₋₇₇ sequence was placed between the U6 promoter sequences and tested for the ability to reduce fluorescence. In both cases, transfected cells exhibited reduced fluorescence when compared to control cells.

### Example 9

### Reduction of mRNA Levels within Cells

The following cassettes were tested for the ability to reduce mRNA levels within cells: p1CLIP_{HP}/HPV16_{C+50-68(H)+GUU}; pSIR (p1CLIP_{S}/HPV16_{C+50-68(H)+GUU} and p1CLIP_{AS}/HPV16_{C+68-50(H)+GUU}); p1CLIP_{S}/HPV16_{C+50-68(H)+GUU}; p2CLIP_{HR}/HPV16₄₇₋₆₈; p2CHOP_{HR}/HPV16₄₇₋₆₈; and pSIR (p1CLIP_{S}/HPV16_{57-77(H)+GUU} and p1CLIP_{AS}/HPV16_{77-57(H)+GUU}). Constructs containing the various cassettes driven by a U6 promoter were co-transfected together with pOG44 constructs (which produce the recombinase) into the Flp-In 293 cell line. Each construct also contained an HPV16 sequence under the control of a CMV promoter. After 30 days of selection, RNA was isolated from the cells and real-time PCR was used to measure HPV16 target RNA levels as described in Pan et al. (Mol. Ther., 7:129-139 (2003)).

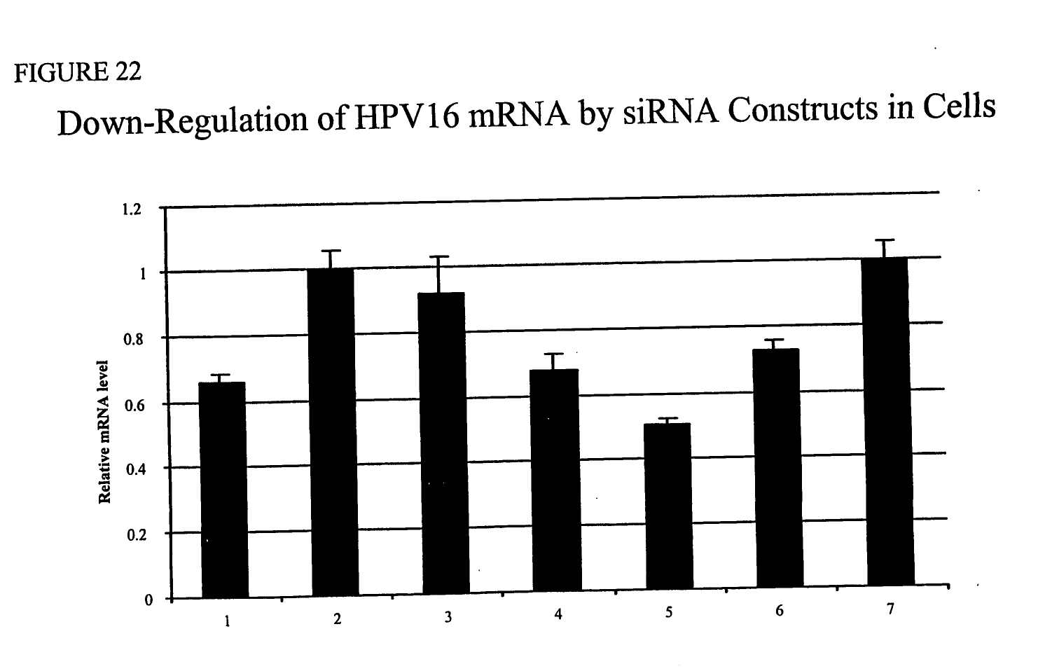

A significant reduction in target mRNA was detected for cells containing the pSIR (p1CLIP_{S}/HPV16_{57-77(H)+GUU} and p1CLIP_{AS}/HPV16_{77-57(H)+GUU}) cassette (FIG. 22). No reduction was detected for cells containing the p1CLIP_{S}/HPV16_{C+50-68(H)+GUU} or pSIR (p1CLIP_{S}/HPV16_{C+50-68(H)+GUU} and p1CLIP_{AS}/HPV16_{C+68-50(H)+GUU}) cassettes. Likewise, no reduction was detected for cells containing the control cassette. The HPV16_{57-77(H)+GUU} sequence contains 21 nucleotides in common with the target, while the HPV16_{C+50-68(H)+GUU} has 19 nucleotides in common with the target. These results demonstrate that the 21-nucleotide sequence is more effective than the 19-nucleotide sequence in inducing RNA interference.

A significant reduction in target mRNA also was detected for cells containing the p2CLIP_{HR}/HPV16₄₇₋₆₈ cassette and for cells containing the p2CHOP_{HR}/HPV16₄₇₋₆₈ cassette. In these cases, the cassettes contained a sequence having one mismatch with respect to the target sequence. These results demonstrate that sequences having a mismatch can be used to induce RNA interference. In addition, the p2CHOP cassette appeared more effective than the p2CLIP cassette. These results demonstrate that the degree of RNA interference can be modulated by altering the ribozyme sequences flanking the sense and/or antisense sequences.

In a separate experiment, the following cassettes were tested for the ability to reduce mRNA levels within cells: p1CLIP_{S}/HPV16_{C+50-68(H)+GUU}; p1CLIP_{AS}/HPV16_{C+68-50(H)+GUU}; pSIR (p1CLIP_{S}/HPV16_{C+50-68(H)+GUU} and p1CLIP_{AS}/HPV16_{C+68-50(H)+GUU}); p1 CLIP_{HP}/HPV16_{C+50-68(H)+GUU}; pSIR (p1CLIP_{HP}/HPV16_{C+50-68(H)+GUU} and p1CLIP_{HP}/HPV16_{C+50-68(H)+GUU}); p2CLIP_{HP}/HPV16_{C+50-68(H)+GUU}; p2CHOP_{HP}/HPV16_{C+50-68(H)+GUU}; pSNIP (p2CLIP_{HP}/HPV16_{C+50-68(H)+GUU} and p2CHOP_{HP}/HPV16_{C+50-68(H)+GUU}); p2CLIP_{HR}/HPV16₄₇₋₆₈; p2CHOP_{HR}/HPV16₄₇₋₆₈; and pSNIP (p2CLIP_{HR}/HPV16₄₇₋₆₈ and p2CHOP_{HR}/HPV16₄₇₋₆₈). Constructs containing the various cassettes driven by a U6 promoter were co-transfected together with pOG44 constructs (which produce the recombinase) into the Flp-In 293 cell line. Each construct also contained an HPV16 sequence under the control of a CMV promoter. One day after transfection, total RNA was isolated from the cells, and quantitative PCR was used to measure HPV16 target RNA levels. The control was cells transfected with an empty pSNIP (p2CLIP and p2CHOP) cassette.

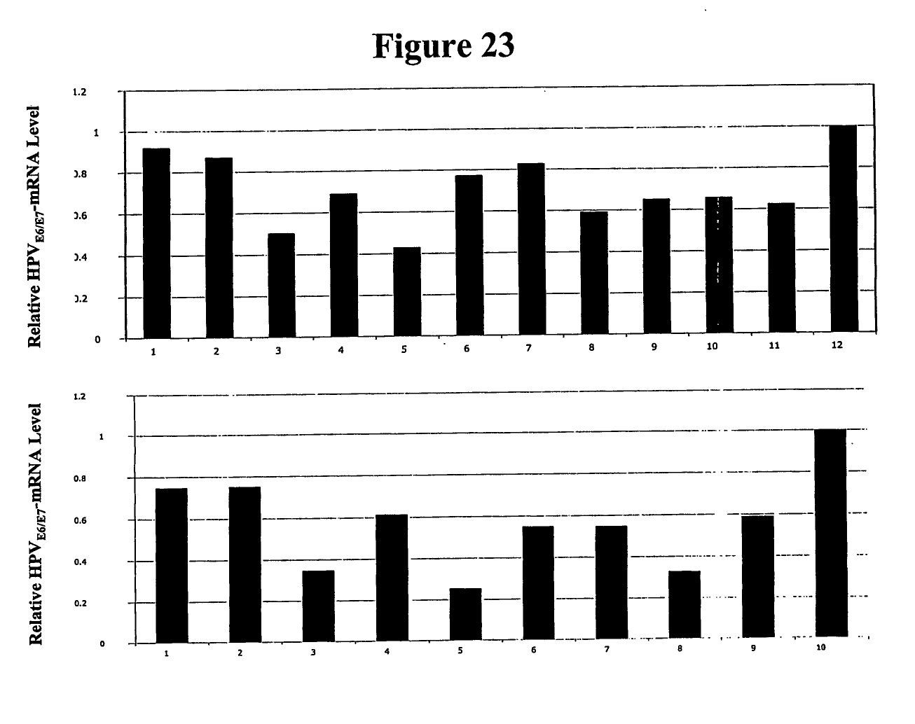

A reduction in target mRNA was detected for cells containing the pSIR (p1CLIP_{S}/HPV16_{C+50-68(H)+GUU} and p1CLIP_{AS}/HPV16_{C+68-50(H)+GUU}); p1CLIP_{HP}/HPV16_{C+50-68(H)+GUU}; pSIR (p1CLIP_{HP}/HPV16_{C+50-68(H)+GUU} and p1CLIP_{HP}/HPV16_{C+50-68(H)+GUU}); pSNIP (p2CLIP_{HP}/HPV16_{C+50-68(H)+GUU} and p2CHOP_{HP}/HPV16_{C+50-68(H)+GUU}); p2CLIP_{HR}/HPV16₄₇₋₆₈; p2CHOP_{HR}/HPV16₄₇₋₆₈; and pSNIP (p2CLIP_{HR}/HPV16₄₇₋₆₈ and p2CHOP_{HR}/HPV16₄₇₋₆₈) cassettes when compared to the levels observed in control cells (FIG. 23). In addition, a detectable reduction was measured for cells containing the p1CLIP_{S}/HPV16_{C+50-68(H)+GUU}; p1CLIP_{AS}/HPV16_{C+68-50(H)+GUU}; p2CLIP_{HP}/HPV16_{C+50-68(H)+GUU}; and p2CHOP_{HP}/HPV16_{C+50-68(H)+GUU} cassettes (FIG. 23).

In another experiment, the following cassettes were tested for the ability to reduce mRNA levels within cells: p1CLIP_{S}/HPV16_{C+50-68(H)+GUU}; p1CLIP_{S}/HPV16_{C+68-50(H)+GUU}; pSIR (p1CLIP_{S}/HPV16_{C+50-68(H)+GUU} and p1CLIP_{AS}/HPV16_{C+68-50(H)+GUU}); p1CLIP_{HP}/HPV16_{C+50-68(H)+GUU}; pSIR (p1CLIP_{HP}/HPV16_{C+50-68(H)+GUU} and p1CLIP_{HP}/HPV16_{C+50-68(H)+GUU}); p2CLIP_{HR}/HPV16₄₇₋₆₈; p2CHOPHR/HPV1647-68; and pSNIP (p2CLIP_{HR}/HPV16₄₇₋₆₈ and p2CHOP_{HR}/HPV16₄₇₋₆₈). Constructs containing the various cassettes driven by a U6 promoter were co-transfected together with pOG44 constructs (which produce the recombinase) into the Flp-In 293 cell line. Each construct also contained an HPV16 sequence under the control of a U6 promoter. Two days after transfection, total RNA was isolated from the cells, and quantitative PCR was used to measure HPV16 target RNA levels. The control was cells transfected with an empty pSNIP (p2CLIP and p2CHOP) cassette. In addition, cells transfected with a cassette containing a pSNIP-type of arrangement with trans-acting rybozymes targeting the target mRNA (as opposed to sense and antisense sequences) were used. This cassette contained one p2CLIP and one p2CHOP, with each being capable of transcribing a trans-acting ribozyme directed against the HPV16₅₀₋₆₈ region.

A reduction in target mRNA was detected for cells containing each cassette (FIG. 23). The largest reduction in target mRNA was observed in cells containing the pSIR (p1CLIP_{S}/HPV16_{C+50-68(H)+GUU} and p1CLIP_{AS}/HPV16_{C+68-50(H)+GUU}); pSIR (p1CLIP_{HP}/HPV16_{C+50-68(H)+GUU} and p1CLIP_{HP}/HPV16_{C+50-68(H)+GUU}); and pSNIP (p2CLIP_{HR}/HPV16₄₇₋₆₈ and p2CHOP_{HR}/HPV16₄₇₋₆₈) cassettes. These results demonstrate that the reductions in target mRNA levels can be greater with respect to control levels when the U6 promoter is used instead of the CMV promoter.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-142. (canceled)

143. An isolated nucleic acid comprising a strand that is a template for an RNA molecule that comprises a sense nucleic acid sequence, an antisense nucleic acid sequence, and a cis-acting ribozyme sequence, wherein said sense nucleic acid sequence is complementary to said antisense nucleic acid sequence, and wherein said sense and antisense nucleic acid sequences form double-stranded RNA upon cleavage of said RNA molecule by said cis-acting ribozyme sequence.

144. The isolated nucleic acid of claim 143, wherein said antisense nucleic acid sequence is complementary to a viral mRNA sequence.

145. The isolated nucleic acid of claim 143, wherein said antisense nucleic acid sequence is complementary to a mammalian mRNA sequence.

146. The isolated nucleic acid of claim 143, wherein said sense nucleic acid sequence is from about 15 to about 50 nucleotides in length.

147. The isolated nucleic acid of claim 143, wherein said sense nucleic acid sequence comprises the sequence as set forth in SEQ ID NO: 1, 2, 3, 4, 5, 6, 43, 44, 45, 46, 47, 48, 49, 50, 51, 52, 53, or 54.

148. The isolated nucleic acid of claim 143, wherein said cis-acting ribozyme sequence is between said sense nucleic acid sequence and said antisense nucleic acid sequence.

149. The isolated nucleic acid of claim 143, wherein said nucleic acid comprises a promoter sequence that promotes transcription of said RNA molecule.

150. The isolated nucleic acid of claim 149, wherein said promoter sequence is a tissue-specific promoter, cell-specific promoter, or pathogen-specific promoter.

151. The isolated nucleic acid of claim 149, wherein said promoter sequence is an H1 promoter sequence or a U6 promoter sequence.

152. The isolated nucleic acid of claim 143, wherein said RNA molecule is transcribed from said nucleic acid when said nucleic acid is within a cell.

153. The isolated nucleic acid of claim 143, wherein said strand is a template for more than one cis-acting ribozyme sequence.

154. The isolated nucleic acid of claim 153, wherein each of said more than one cis-acting ribozyme sequence is different.

155. An isolated nucleic acid comprising a strand that is a template for an RNA molecule that comprises a sense nucleic acid sequence, an antisense nucleic acid sequence, and a cis-acting ribozyme sequence, wherein said sense nucleic acid sequence is complementary to said antisense nucleic acid sequence, wherein said sense and antisense nucleic acid sequences form a single-stranded RNA upon cleavage of said RNA molecule by said cis-acting ribozyme sequence, and wherein said single-stranded RNA contains no more than one hairpin loop structure and lacks RNA cleaving activity.

156. The isolated nucleic acid of claim 155, wherein said nucleic acid is double stranded.

157. The isolated nucleic acid of claim 155, wherein said nucleic acid is single stranded.

158. The isolated nucleic acid of claim 155, wherein said strand is a template for more than one cis-acting ribozyme sequence.

159. The isolated nucleic acid of claim 158, wherein each of said more than one cis-acting ribozyme sequence is different.

160. A method of identifying sequences capable of inducing RNA interference against a target mRNA, said method comprising:
(a) introducing a vector preparation into cells, wherein each vector of said vector preparation comprises:
(1) a target nucleic acid sequence, wherein said target nucleic acid sequence is a template for said target mRNA;
(2) a reporter nucleic acid sequence, wherein said reporter nucleic acid sequence encodes a polypeptide, and wherein said target nucleic acid sequence and said reporter nucleic acid sequence are transcribed as a single fusion mRNA; and
(3) a promoter sequence region, wherein said promoter sequence region comprises: (i) a member of a plurality of test nucleic acid sequences, and (ii) two promoter sequences operably linked to said member in an arrangement that promotes transcription of both strands of said member;
(b) identifying at least one cell lacking said polypeptide; and
(c) obtaining the sequence of said member from said cell identified in step (b), thereby identifying said sequence as being capable of inducing RNA interference against said target mRNA.

161. The method of claim 160, wherein said polypeptide is a fluorescent polypeptide.

162. The method of claim 160, wherein said polypeptide is lethal to said cell.
